(19) United States
(12) Patent Application Publication
(10) Pub. No.: US 2023/0020036 A1
(43) Pub. Date: Jan. 19, 2023

(54) TREATMENT OF EPILEPTIC CONDITIONS WITH GABAA RECEPTOR MODULATORS

(71) Applicant: NEUROCYCLE THERAPEUTICS, INC., San Diego, CA (US)

(72) Inventors: Jed HUBBS, San Diego, CA (US); Matthew TOCZKO, San Diego, CA (US)

(21) Appl. No.: 17/770,794

(22) PCT Filed: Oct. 23, 2020
(86) PCT No.: PCT/US2020/057104

§ 371 Date: Apr. 21, 2022

Related U.S. Application Data
(60) Provisional application No. 62/925,081, filed on Oct. 23, 2019.
(60) Provisional application No. 62/950,674, filed on Dec. 19, 2019.

(51) Int. Cl. A61K 31/5517; A61P 25/08; A61K 31/53

(52) U.S. Cl. CPC A61K 31/5517; A61P 25/08; A61K 31/53

(57) ABSTRACT

Disclosed herein are GABA_{A} receptor modulators and compositions comprising GABA_{A} receptor modulators for treatment of epileptic conditions. Also disclosed herein are methods of treating epileptic conditions in a subject by administering a GABA_{A} receptor modulators or composition as described herein.

### CROSS-REFERENCE

This application claims the benefit of U.S. Provisional Application No. 62/925,081, filed on Oct. 23, 2019, and 62/950,674, filed on Dec. 19, 2019, each of which is hereby incorporated by reference in its entirety.

### STATEMENT AS TO FEDERALLY SPONSORED RESEARCH

This invention was made with government support under Award Number R43NS107051 awarded by the National Institute of Neurological Disorders and Stroke of the National Institutes of Health. The government has certain rights in the invention.

### SUMMARY

Disclosed herein are methods of treating an epileptic condition in a subject, comprising administering to the subject a therapeutically effective amount of a compound of a general formula (1a), general formula (1 b) or general formula (1c),

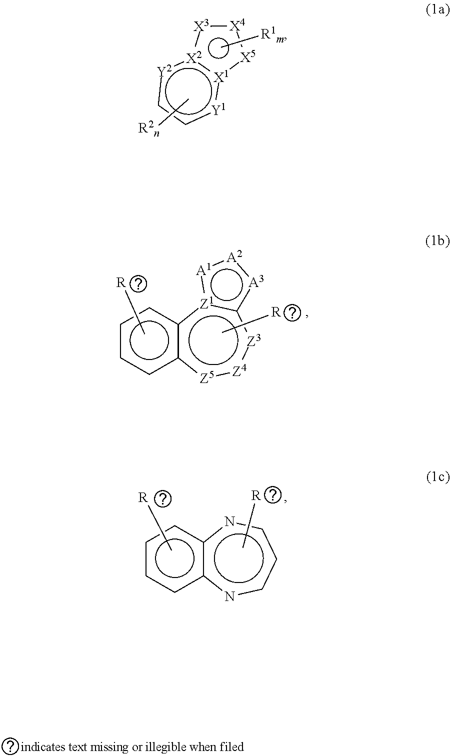

wherein X¹, X², X³, X⁴ and X⁵ are independently —C, —N, —S or —O wherein at least two of X¹, X², X³, X⁴ and X⁵ are —N, Y¹ and Y² are independently —C or —N, m of R¹ₘ is 1, wherein R¹ is an unsubstituted phenyl, a phenyl substituted with C₁-C₄-alkyl, F, Cl, Br, I, —CN, a substituted or unsubstituted biphenyl or —(C═O)—R³, wherein R³ is a substituted or unsubstituted aryl or 5- to 6-membered heteroaryl, n of R²ₙ is 1 or 2, wherein each R² is independently a substituted or unsubstituted C₃-C₈ cycloalkyl, a substituted or unsubstituted C₁-C₆ alkyl, a substituted or unsubstituted C₁-C₆ alcohol, a substituted or unsubstituted 6-membered heteroaryl, a halogen, or —O—CH₂—R⁴, wherein R⁴ is a substituted or unsubstituted 5- or 6-membered heteroaryl, Z¹, Z³, Z⁴, and Z⁵ are independently —C, —N, —S or —O, A¹, and A² and A³, are independently —C, —N, or —(C═O)—O—R⁷, or

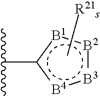

wherein R⁷ is alkyl, B¹, B², B³, and B⁴ are independently —C, —N, or —O, s of R²¹ₛ is 1, 2, 3 or 4, and l of R⁵ₗ is 1 or 2, wherein each R⁵ is independently a C₁-C₄ alkynyl or a halogen, k of R⁶ₖ is 1, 2, 3 or 4, wherein each R⁶ is independently a substituted or unsubstituted aryl, a substituted or unsubstituted heteroaryl, a substituted or unsubstituted C₁-C₃ alkyl, or hydrogen, p of R¹²ₚ is 1 or 2, wherein each R¹² is independently a substituted or unsubstituted C₁-C₄-alkyl, I, Br, Cl or F, and q of R¹³_{q} is 1, 2, 3 or 4, wherein each R¹³ is independently a substituted or unsubstituted aryl, a substituted or unsubstituted heteroaryl, a substituted or unsubstituted C₁-C₃ alkyl, oxygen or hydrogen, to treat the epileptic condition in the subject. In some embodiments, the compound is of the general formula (2), (3), (4), (5), (1c) or (7),

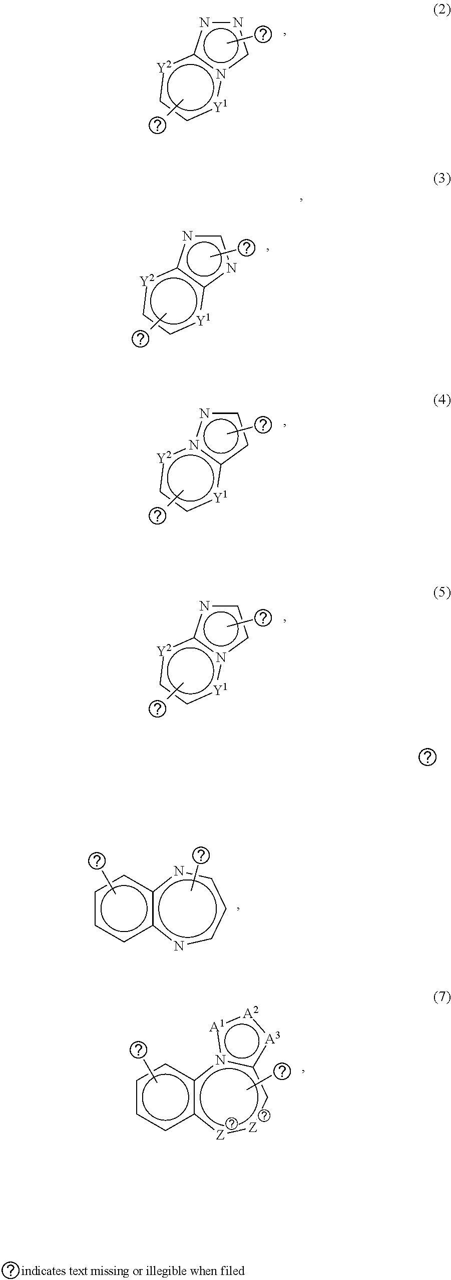

In some embodiments, the compound is of the general formula (2a), (3a), (4a), (5a), (5b), (1c) or (7a)

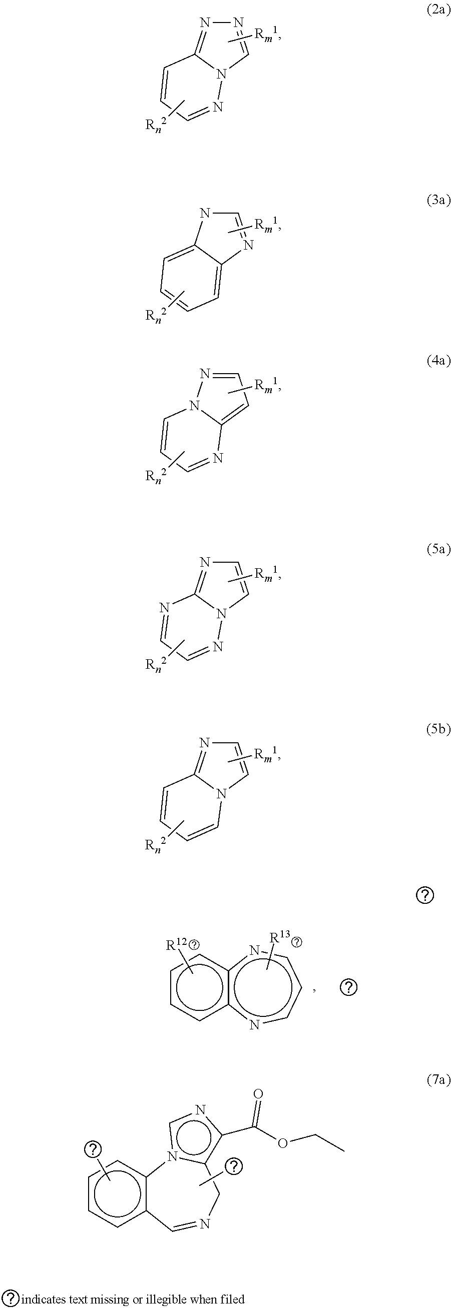

In some embodiments, m of R¹ is 1, and wherein R¹ is: an unsubstituted phenyl, a substituted phenyl comprising C₁-C₄-alkyl, F, Cl, Br, I, —CN as substituents, an unsubstituted biphenyl, a substituted biphenyl comprising at least one —CN as a substituent, a substituted biphenyl comprising at least one —CN as a substituent, or —(C═O)—R³, wherein R³ is pyridine. In some embodiments, the compound is of the general formula (2a′), (3a′), (4a′), (5a′), (5b′), (VI) or (7a′),

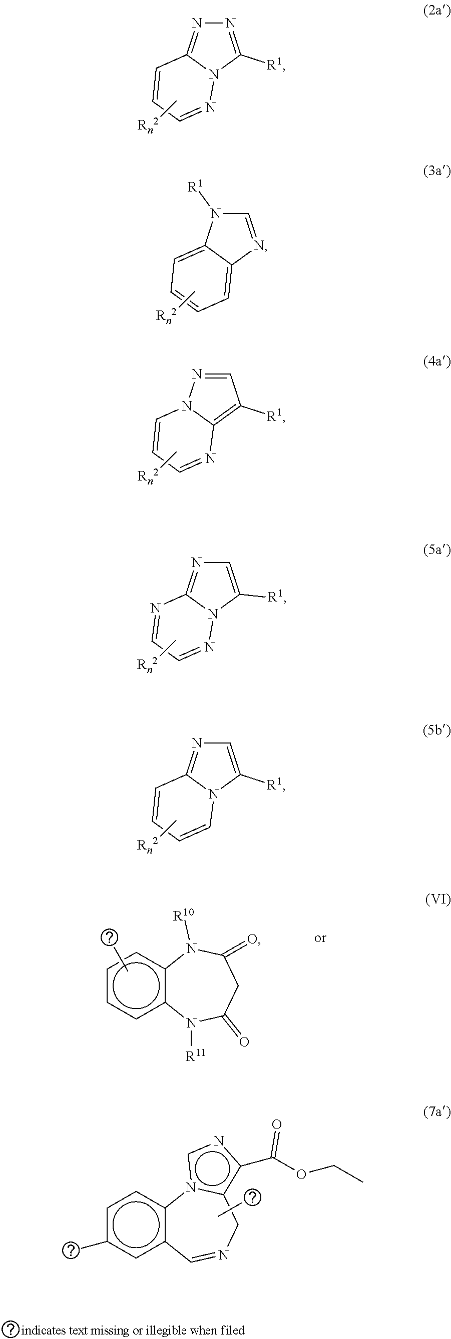

wherein R₁₀ is a substituted or unsubstituted aryl, a substituted or unsubstituted C₁-C₃ alkyl or hydrogen, R₁₁ is a substituted or unsubstituted aryl, a substituted or unsubstituted C₁-C₃ alkyl, or hydrogen, or p of R¹²ₚ is 1 and R¹² is I, Br, Cl or F. In some embodiments, the compound is of the general formula (2a″), (3a″), (4a″), (5a″), (5b″), (VIa″) or (7a″),

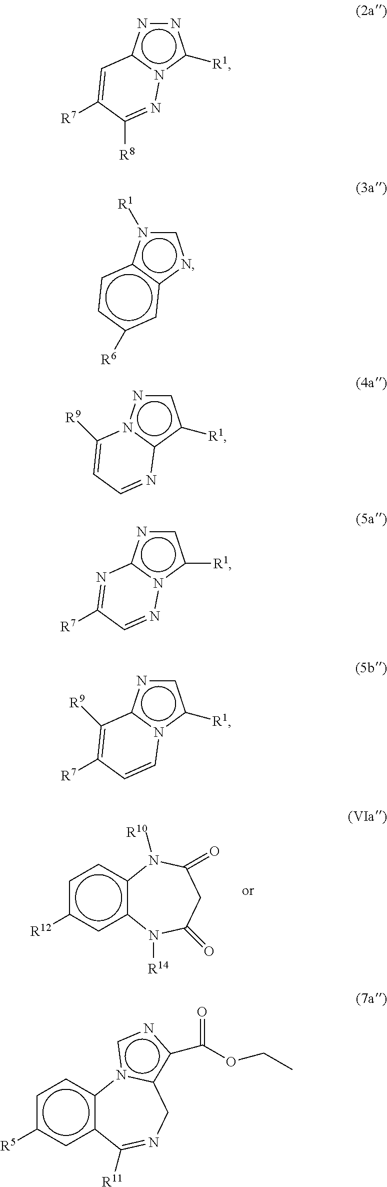

wherein R⁷ is: an unsubstituted C₁-C₆ alkyl, an unsubstituted C₃-C₈ cycloalkyl, an unsubstituted C₁-C₆ alcohol, R⁸ is: —O—CH₂—R⁴, wherein R⁴ is a substituted or unsubstituted 5-membered heteroaryl, or an unsubstituted C₁-C₆ alcohol, R⁹ is: an unsubstituted C₆ heteroaryl, or a halogen, or R⁹ is an unsubstituted 6-membered heteroaryl in formula (4a″) or a halogen in formula (5b″), R¹⁰ is a C₁-C₃ alkyl or hydrogen, R¹¹ is a substituted or unsubstituted aryl or heteroaryl, R¹⁴ is a substituted or unsubstituted aryl or heteroaryl, R¹² is I, Cl, Br or F, or R⁵ is C₂ alkynyl or I. In some embodiments, the compound is an α1, α2, α3, or α5 GABA_{A} receptor modulator. In some embodiments, the compound is a positive, allosteric α2 or α3 GABA_{A} receptor modulator. In some embodiments, the subject is a human. In some embodiments, the subject is a dog. In some embodiments, the subject is aged 0-17 years old. In some embodiments, the subject is aged 18-130 years old. In some embodiments, the therapeutically effective amount of the compound is present in a pharmaceutical composition that comprises a pharmaceutically acceptable excipient, diluent, or carrier. In some embodiments, the epileptic condition is selected from the group consisting of: Benign centrotemporal lobe epilepsy of childhood, Benign occipital epilepsy of childhood (BOEC), Autosomal dominant nocturnal frontal lobe epilepsy (ADNFLE), Primary reading epilepsy, Childhood absence epilepsy (CEA), Juvenile absence epilepsy, Juvenile myoclonic epilepsy (JME), Symptomatic localization-related epilepsies, Temporal lobe epilepsy (TLE), Frontal lobe epilepsy, Rasmussen's encephalitis, West syndrome, Dravet syndrome, Progressive myoclonic epilepsies, and Lennox-Gastaut syndrome (LGS).

Also disclosed herein are methods of treating epilepsy associated with a mutation in a sodium channel in a subject comprising administering to the subject a compound of the general formula (1a), general formula (1b) or general formula (1c),

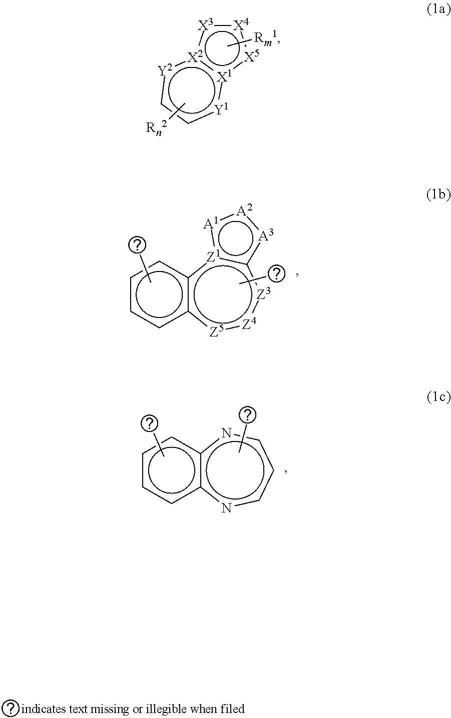

Wherein X¹, X², X³, X⁴ and X⁵ are independently —C, —N, —S or —O wherein at least two of X¹, X², X³, X⁴ and X⁵ are —N, Y¹ and Y² are independently —C or —N, m of R¹ₘ is 1, wherein R¹ is an unsubstituted phenyl, a phenyl substituted with C₁-C₄-alkyl, F, Cl, Br, I, —CN, a substituted or unsubstituted biphenyl or —(C═O)—R³, wherein R³ is a substituted or unsubstituted aryl or 5- to 6-membered heteroaryl, n of R²ₙ is 1 or 2, wherein each R² is independently a substituted or unsubstituted C₃-C₈ cycloalkyl, a substituted or unsubstituted C₁-C₆ alkyl, a substituted or unsubstituted C₁-C₆ alcohol, a substituted or unsubstituted 6-membered heteroaryl, a halogen, or —O—CH₂—R⁴, wherein R⁴ is a substituted or unsubstituted 5- or 6-membered heteroaryl, Z¹, Z³, Z⁴, and Z⁵ are independently —C, —N, —S or —O, A¹, and A² and A³, are independently —C, —N, or —(C═O)—O—R⁷, or,

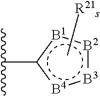

wherein R⁷ is alkyl, B¹, B², B³, and B⁴ are independently —C, —N, or O—, s of R²¹ₛ is 1, 2, 3 or 4, and l of R⁵ₗ is 1 or 2, wherein each R⁵ is independently a C₁-C₄ alkynyl or a halogen, k of R⁶ₖ is 1, 2, 3 or 4, wherein each R⁶ is independently a substituted or unsubstituted aryl, a substituted or unsubstituted heteroaryl, a substituted or unsubstituted C₁-C₃ alkyl, or hydrogen, p of R¹² is 1 or 2, wherein each R¹² is independently a substituted or unsubstituted C₁-C₄-alkyl, I, Br, Cl or F, and q of R¹³ is 1, 2, 3 or 4, wherein each R¹³ is independently a substituted or unsubstituted aryl, a substituted or unsubstituted heteroaryl, a substituted or unsubstituted C₁-C₃ alkyl, oxygen or hydrogen, to treat the epilepsy associated with the mutation in the sodium channel. In some embodiments, the compound is of the general formula (2), (3), (4), (5), (1c) or (7),

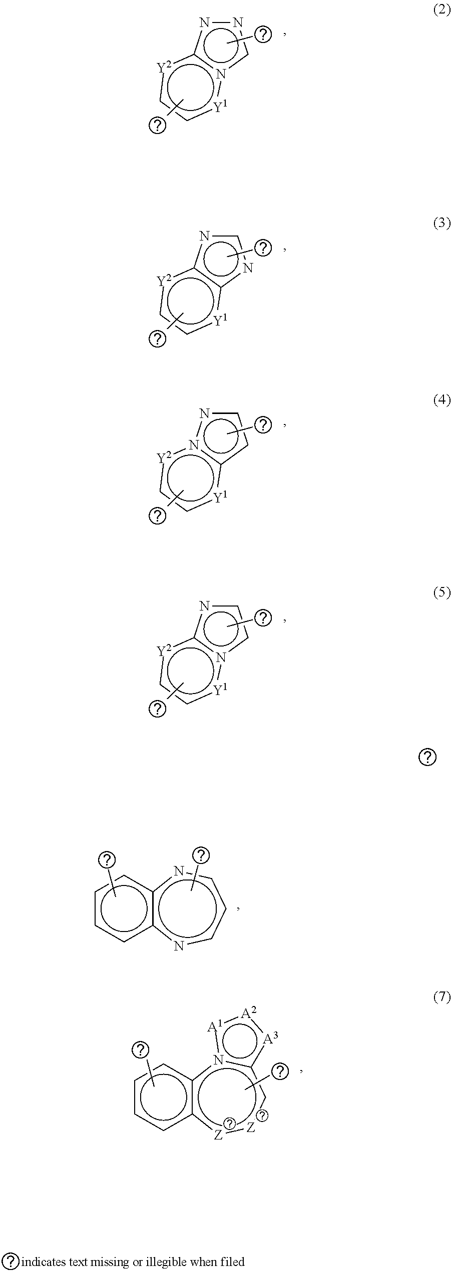

In some embodiments, the compound is of the general formula (2a), (3a), (4a), (5a), (5b), (1c) or (7a)

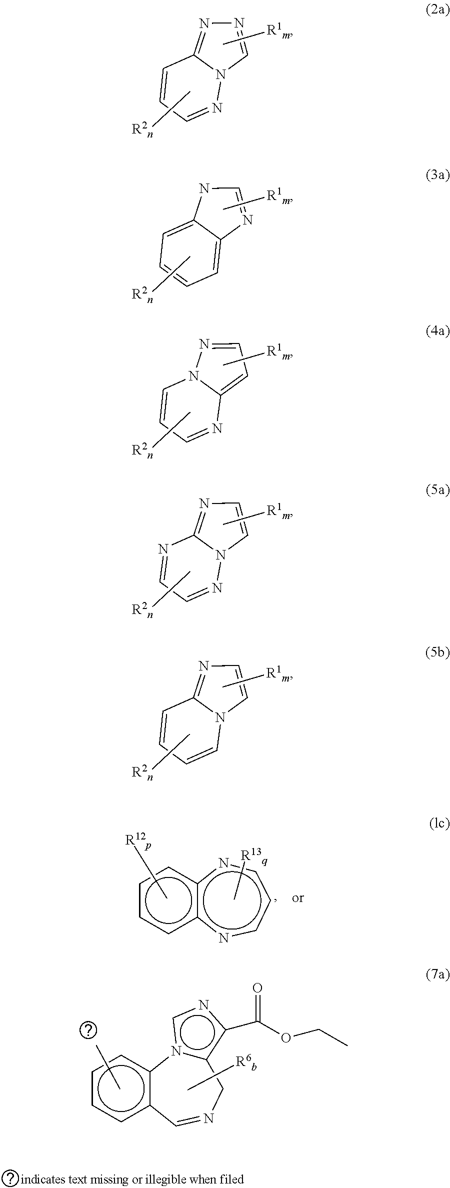

In some embodiments, m of R¹ₘ is 1, and wherein R¹ is: an unsubstituted phenyl, a substituted phenyl comprising C₁-C₄-alkyl, F, Cl, Br, I, —CN as substituents, an unsubstituted biphenyl, a substituted biphenyl comprising at least one —CN as a substituent, a substituted biphenyl comprising at least one —CN as a substituent, or —(C═O)—R³, wherein R³ is pyridine. In some embodiments, the compound is of the general formula (2a′), (3a′), (4a′), (5a′), (5b′), (VI) or (7a′),

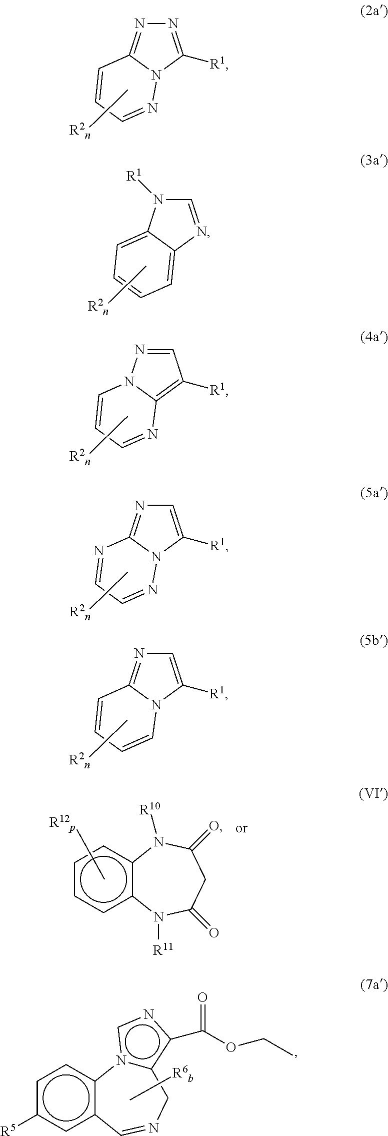

wherein R₁₀ is a substituted or unsubstituted aryl, a substituted or unsubstituted C₁-C₃ alkyl or hydrogen, Ru is a substituted or unsubstituted aryl, a substituted or unsubstituted C₁-C₃ alkyl, or hydrogen, or p of R²ₚ is 1 and R¹² is I, Br, Cl or F. In some embodiments, the compound is of the general formula (2a″), (3a″), (4a″), (5a″), (5b″), (VIa″) or (7a″),

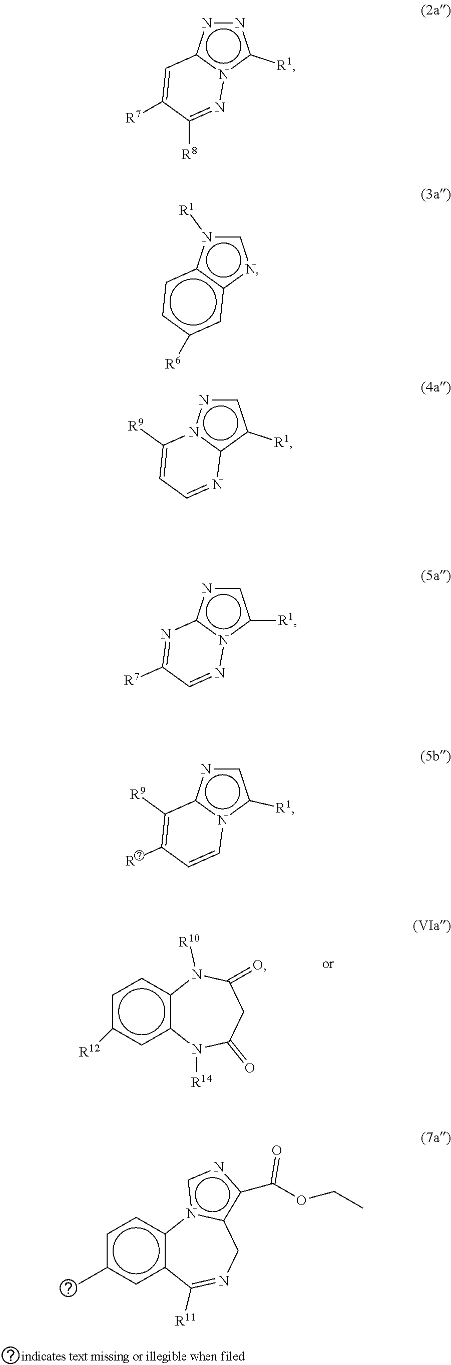

wherein R⁷ is: an unsubstituted C₁-C₆ alkyl, an unsubstituted C₃-C₈ cycloalkyl, an unsubstituted C₁-C₆ alcohol, R⁸ is: —O—CH₂—R⁴, wherein R⁴ is a substituted or unsubstituted 5-membered heteroaryl, or an unsubstituted C₁-C₆ alcohol, R⁹ is: an unsubstituted C₆ heteroaryl, or a halogen, or R⁹ is an unsubstituted 6-membered heteroaryl in formula (4a″) or a halogen in formula (5b″), R¹⁰ is a C₁-C₃ alkyl or hydrogen, R¹¹ is a substituted or unsubstituted aryl or heteroaryl, R¹⁴ is a substituted or unsubstituted aryl or heteroaryl, R¹² is I, Cl, Br or F, or R⁵ is C₂ alkynyl or I. In some embodiments, the compound is an α1, α2, α3, or α5 GABA_{A} receptor modulator. In some embodiments, the compound is a positive, allosteric α2 or α3 GABA_{A} receptor modulator. In some embodiments, the subject is a human. In some embodiments, the subject is a dog. In some embodiments, the subject is aged 0-17 years old. In some embodiments, the subject is aged 18-130 years old. In some embodiments, the therapeutically effective amount of the compound is present in a pharmaceutical composition that comprises a pharmaceutically acceptable excipient, diluent, or carrier. In some embodiments, the mutation in the sodium channel comprises a mutation in a sodium voltage-gated channel alpha subunit 1 (SCN1A) gene.

Also disclosed herein are methods of treating Dravet Syndrome in a subject comprising administering to the subject an amount of a pharmaceutical composition comprising a compound selected from the group consisting of:

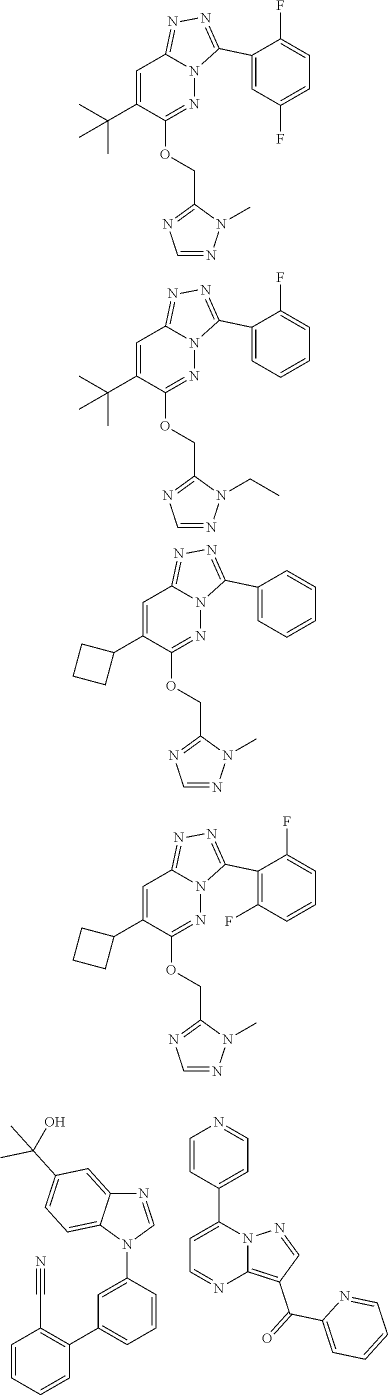

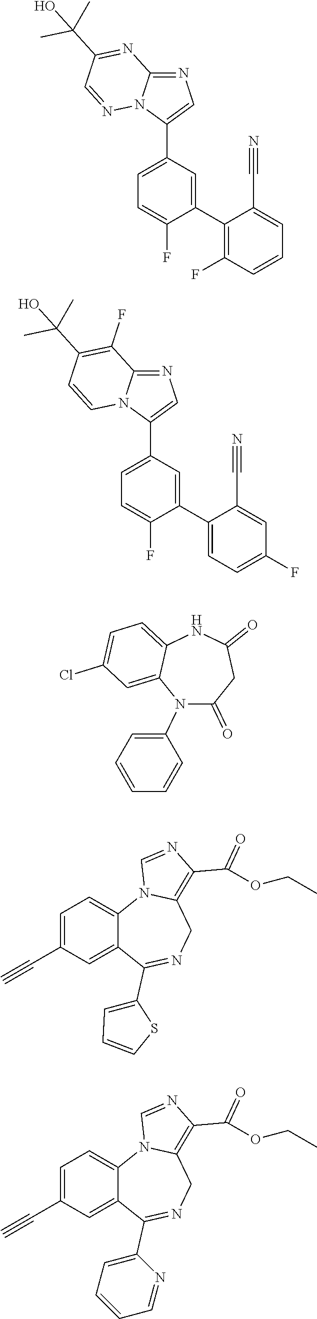

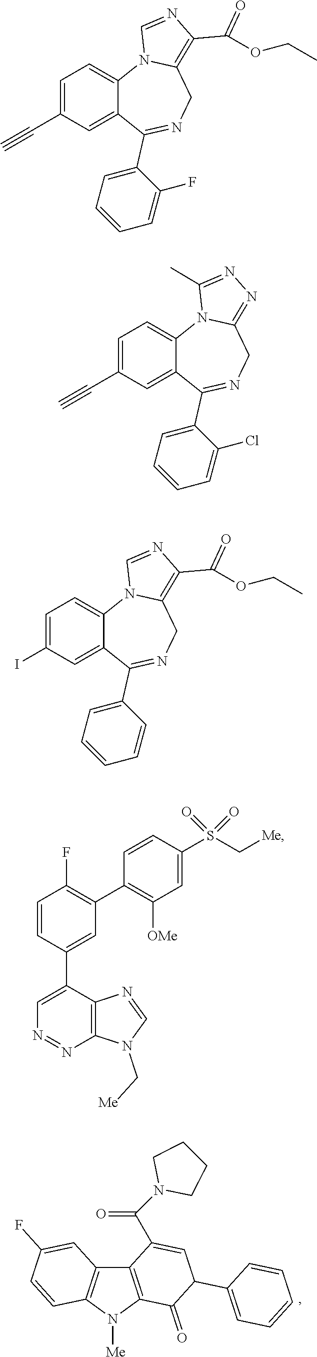

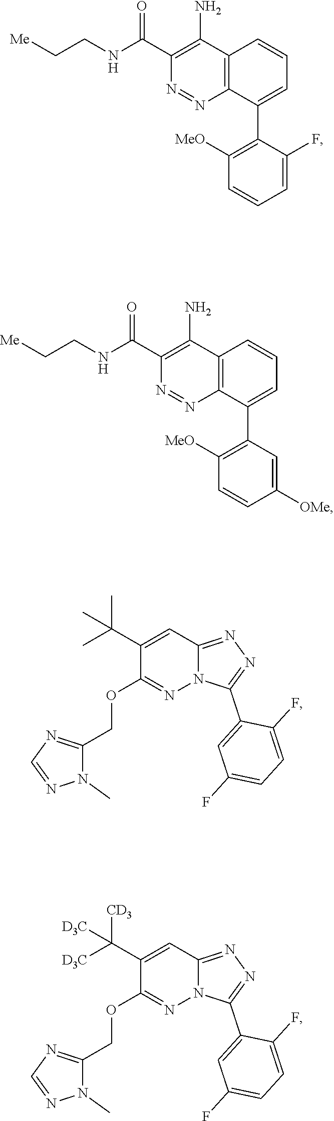 [SMILES: CCCNC(=O)c1nnc2c(-c3c(F)cccc3OC)cccc2c1N ; CCCNC(=O)c1nnc2c(-c3cc(OC)ccc3OC)cccc2c1N ; Cn1ncnc1COc1nn2c(-c3cc(F)ccc3F)nnc2cc1C(C)(C)C ; [2H]C([2H])([2H])C(c1cc2nnc(-c3cc(F)ccc3F)n2nc1OCc1ncnn1C)(C([2H])([2H])[2H])C([2H])([2H])[2H]]

a salt thereof, and a polymorph thereof, to treat the Dravet Syndrome in the subject. In some embodiments, the compound is:

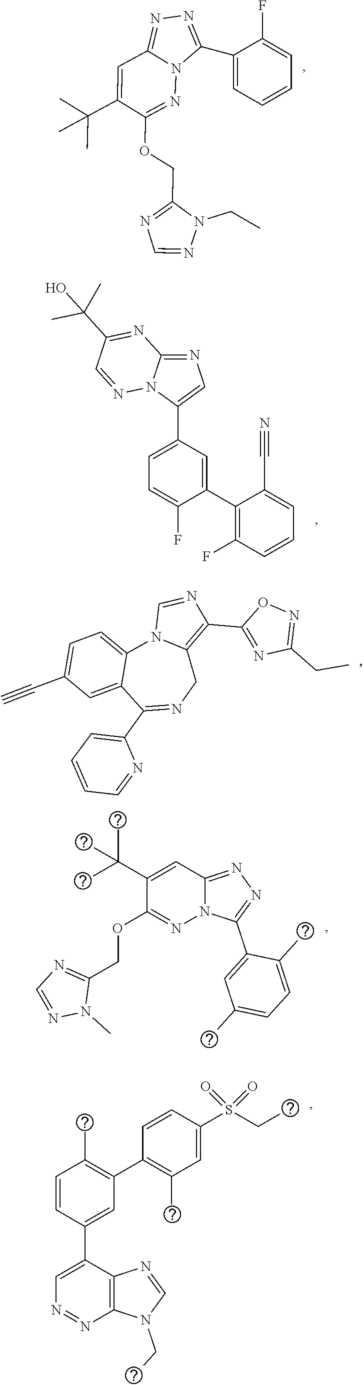

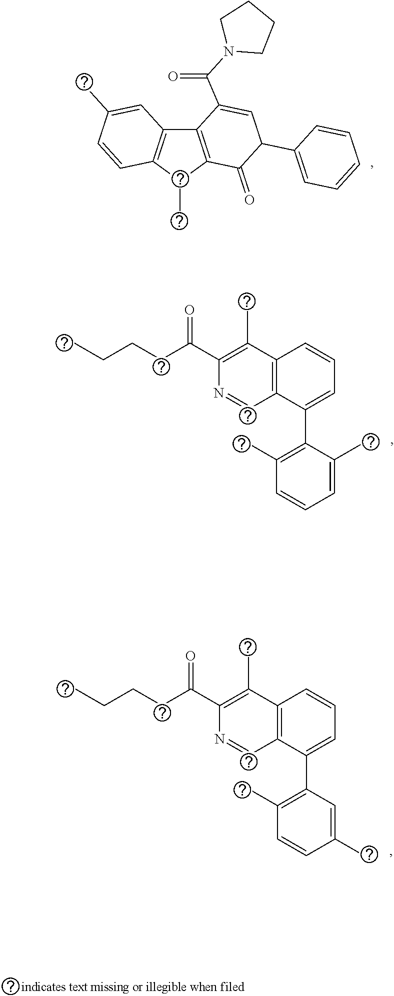

a salt thereof, or a polymorph thereof. In some embodiments, the amount is effective to treat the Dravet Syndrome when administered at a dose of from about 0.003 mg/kg per day to about 10 mg/kg (e.g., from about 0.003 mg/kg to about 100 mg/kg, from about 0.003 mg/kg to about 95 mg/kg, from about 0.003 mg/kg to about 90 mg/kg, from about 0.003 mg/kg to about 85 mg/kg, from about 0.003 mg/kg to about 80 mg/kg, from about 0.003 mg/kg to about 75 mg/kg, from about 0.003 mg/kg to about 70 mg/kg, from about 0.003 mg/kg to about 65 mg/kg, from about 0.003 mg/kg to about 60 mg/kg, from about 0.003 mg/kg to about 55 mg/kg, from about 0.003 mg/kg to about 50 mg/kg, from about 0.003 mg/kg to about 45 mg/kg, from about 0.003 mg/kg to about 40 mg/kg, from about 0.003 mg/kg to about 35 mg/kg, from about 0.003 mg/kg to about 30 mg/kg, from about 0.003 mg/kg to about 25 mg/kg, from about 0.003 mg/kg to about 20 mg/kg, from about 0.003 mg/kg to about 15 mg/kg, from about 0.003 mg/kg to about 10 mg/kg, from about 0.003 mg/kg to about 9 mg/kg, from about 0.003 mg/kg to about 8 mg/kg, from about 0.003 mg/kg to about 7 mg/kg, from about 0.003 mg/kg to about 6 mg/kg, from about 0.003 mg/kg to about 5 mg/kg, from about 0.003 mg/kg to about 4 mg/kg, from about 0.003 mg/kg to about 3 mg/kg, from about 0.003 mg/kg to about 2 mg/kg, or from about 0.003 mg/kg to about 1 mg/kg) per day of a body weight of a subject when administered to the subject. In some embodiments, the subject is a human. In some embodiments, the subject is a dog. In some embodiments, the pharmaceutical composition further comprises a pharmaceutically acceptable excipient, diluent, or carrier. In some embodiments, the pharmaceutical composition comprises the carrier, wherein the carrier is methyl cellulose. In some embodiments, the compound is

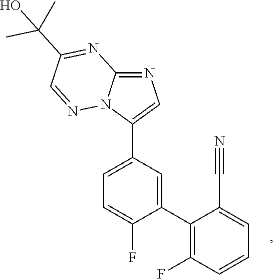 [SMILES: CC(C)(O)c1cnn2c(-c3ccc(F)c(-c4c(F)cccc4C#N)c3)cnc2n1]

a polymorph thereof, or a salt thereof. In some embodiments, the pharmaceutical composition comprises the salt of the compound. In some embodiments, the salt is a phosphate salt. In some embodiments, the salt is a sulfate salt. In some embodiments, the pharmaceutical composition comprises the polymorph of the compound. In some embodiments, the polymorph has an X-ray powder diffraction (XRPD) having characteristic peak locations of at least three of the values selected from the group consisting of: about 6.4, 7.5, 10.2, 12.7, 13.3, 14.5, 16.0, 17.1, 17.4, 17.9, 18.5, 19.1, 19.7, 20.3, 20.9, 21.5, 22.6, 23.7, 26.2, 26.7, 26.9, 27.5, 28.4, 30.2, and 32.1±0.2 degrees, 2-Theta, when measured using: (a) an X-ray wavelength parameter of Cu: K-Alpha (λ=1.54179 Å); (b) an X-Ray tube voltage setting of 40 kV and current of 40 mA; (c) a scan scope of from about 3 degrees to about 40 degrees; (d) a sample rotation speed of about 15 rpm; and (e) a scanning rate of 10 degrees per minute. In some embodiments, the administering comprises an oral administration. In some embodiments, the administering is performed at least once a day. In some embodiments, the administering of the amount that is effective to treat Dravet Syndrome does not produce drowsiness or sedation in the subject.

Also disclosed herein are methods of treating an epileptic condition in a subject comprising administering to the subject an amount of a compound of formula:

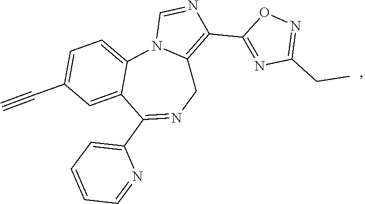 [SMILES: C#Cc1ccc2c(c1)C(c1ccccn1)=NCc1c(-c3nc(CC)no3)ncn1-2]

or a pharmaceutically acceptable salt, or polymorph thereof, to treat the epileptic condition in the subject, wherein the amount comprises a dose of from about 0.003 mg/kg to about 10 mg/kg (i.e. from about 0.003 mg/kg to about 100 mg/kg, from about 0.003 mg/kg to about 95 mg/kg, from about 0.003 mg/kg to about 90 mg/kg, from about 0.003 mg/kg to about 85 mg/kg, from about 0.003 mg/kg to about 80 mg/kg, from about 0.003 mg/kg to about 75 mg/kg, from about 0.003 mg/kg to about 70 mg/kg, from about 0.003 mg/kg to about 65 mg/kg, from about 0.003 mg/kg to about 60 mg/kg, from about 0.003 mg/kg to about 55 mg/kg, from about 0.003 mg/kg to about 50 mg/kg, from about 0.003 mg/kg to about 45 mg/kg, from about 0.003 mg/kg to about 40 mg/kg, from about 0.003 mg/kg to about 35 mg/kg, from about 0.003 mg/kg to about 30 mg/kg, from about 0.003 mg/kg to about 25 mg/kg, from about 0.003 mg/kg to about 20 mg/kg, from about 0.003 mg/kg to about 15 mg/kg, from about 0.003 mg/kg to about 10 mg/kg, from about 0.003 mg/kg to about 9 mg/kg, from about 0.003 mg/kg to about 8 mg/kg, from about 0.003 mg/kg to about 7 mg/kg, from about 0.003 mg/kg to about 6 mg/kg, from about 0.003 mg/kg to about 5 mg/kg, from about 0.003 mg/kg to about 4 mg/kg, from about 0.003 mg/kg to about 3 mg/kg, from about 0.003 mg/kg to about 2 mg/kg, or from about 0.003 mg/kg to about 1 mg/kg). In some embodiments, the administering reduces an amount of seizures to an amount that is at least 20% less than an amount of seizures that occurs based on an administering of an equivalent dose of clobazam. In some embodiments, the amount comprises a dose of from about 0.08 mg/kg to about 2.5 mg/kg. In some embodiments, the compound is present in a shelf stable formulation. In some embodiments, the compound is formulated as a non-drowsy formulation. In some embodiments, the non-drowsy formulation comprises caffeine. In some embodiments, the compound, the pharmaceutically acceptable salt, or polymorph thereof, is a phosphate salt or polymorph thereof. In some embodiments, the compound, the pharmaceutically acceptable salt, or polymorph thereof, is a sulfate salt or polymorph thereof.

Described herein is a method of treating an epileptic condition in a subject, comprising administering to the subject a pharmaceutical composition that comprises a compound of the general formula (5a′) or a salt or polymorph thereof,

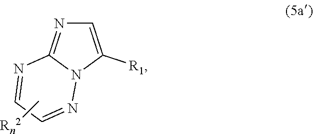

wherein
   R¹ is an unsubstituted phenyl, a phenyl substituted with C₁-C₄-alkyl, F, Cl, Br, I, —CN, a substituted or unsubstituted biphenyl, or —(C═O)—R³ wherein R³ is a substituted or unsubstituted aryl or 5- to 6-membered heteroaryl, and
   n of R²ₙ is 1 or 2, wherein each R² is independently a substituted or unsubstituted C₃-C₈ cycloalkyl, a substituted or unsubstituted C₁-C₆ alkyl, a substituted or unsubstituted C₁-C₆ alcohol, a substituted or unsubstituted 6-membered heteroaryl, a halogen, or —O—CH₂—R⁴, wherein R⁴ is a substituted or unsubstituted 5- or 6-membered heteroaryl,
wherein the administering is in an amount effective to treat the epileptic condition in the subject, and wherein the amount comprises a dose of the compound or a salt or polymorph thereof of from about 0.003 mg to about 1 mg per kg of a body weight of the subject per day. Described herein is a method of treating an epileptic condition in a subject, comprising administering to the subject a pharmaceutical composition that comprises a compound having a structure of

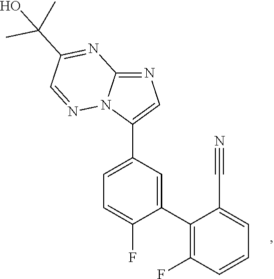 [SMILES: CC(C)(O)c1cnn2c(-c3ccc(F)c(-c4c(F)cccc4C#N)c3)cnc2n1]

or a salt or polymorph thereof, wherein the administering is in an amount effective to treat the epileptic condition in the subject, and wherein the amount comprises a dose of the compound or a salt or polymorph thereof of from about 0.0003 mg to about 1 mg per kg of a body weight of the subject per day. In some embodiments, the epileptic condition is a general epilepsy or a genetic epilepsy. In some embodiments, the epileptic condition is Dravet syndrome. In some embodiments, the epileptic condition is a focal seizure. In some embodiments, the epileptic condition is selected from the group consisting of: Benign centrotemporal lobe epilepsy of childhood, Benign occipital epilepsy of childhood (BOEC), Autosomal dominant nocturnal frontal lobe epilepsy (ADNFLE), Primary reading epilepsy, Childhood absence epilepsy (CEA), Juvenile absence epilepsy, Juvenile myoclonic epilepsy (JME), Symptomatic localization-related epilepsies, Temporal lobe epilepsy (TLE), Frontal lobe epilepsy, Rasmussen's encephalitis, Cerebral palsy, Cerebral hypoxia, Down's syndrome, hypoxic-ischemic encephalopathy (HIE), West syndrome, Dravet syndrome, focal seizures, Progressive myoclonic epilepsies, focal seizures, or Lennox-Gastaut syndrome (LGS). In some embodiments, the compound, or the salt or polymorph thereof, is a phosphate salt or polymorph thereof. In some embodiments, the compound, or the salt or polymorph thereof, is a sulfate salt or polymorph thereof. In some embodiments, the compound, or a salt or polymorph thereof, is a phosphate polymorph, and wherein the phosphate polymorph exhibits an X-ray powder diffraction (XRPD) pattern having characteristic peak locations of at least three of the values selected from the group consisting of: about 6.4, 7.5, 10.2, 12.7, 13.3, 14.5, 16.0, 17.1, 17.4, 17.9, 18.5, 19.1, 19.7, 20.3, 20.9, 21.5, 22.6, 23.7, 26.2, 26.7, 26.9, 27.5, 28.4, 30.2, and 32.1±0.2 degrees, 2-theta, when measured using: (a) an X-ray wavelength parameter of Cu: K-Alpha (λ=1.54179 Å); (b) an X-Ray tube voltage setting of 40 kV and current of 40 mA; (c) a scan scope of from about 3 degrees to about 40 degrees; (d) a sample rotation speed of about 15 rpm; and (e) a scanning rate of 10 degrees per minute. In some embodiments, the pharmaceutical composition is formulated for oral or transdermal administration. In some embodiments, the amount comprises a dose of the compound or a salt or polymorph thereof of less than 0.3 mg per kg of the body weight of the subject per day. In some embodiments, the amount comprises a dose of the compound or a salt or polymorph thereof of less than 0.1 mg per kg of the body weight of the subject per day. In some embodiments, the amount comprises a dose of the compound or a salt or polymorph thereof of less than 0.03 mg per kg of the body weight of the subject per day. In some embodiments, the epileptic condition is Dravet syndrome. In some embodiments, the epileptic condition is a focal seizure. In some embodiments, the compound, or a salt or polymorph thereof, is not a phosphate salt or polymorph thereof, and is not a sulfate salt or polymorph thereof. In certain embodiments, the subject is a human subject. In some embodiments, the compound is formulated as a non-drowsy formulation. In some embodiments, the non-drowsy formulation comprises caffeine. In some embodiments, the subject is a human.

### BRIEF DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which the principles of exemplary embodiments are utilized, and the accompanying drawings of which:

FIG. 1 depicts the starting body temperature (mean (±SEM)) of pretreated female mice prior to onset of temperature induced seizures. The average body temperature of the female mice ranged from about 34.5° C. to about 35.5° C. prior to the inducement of the heat induced seizure.

FIG. 2 depicts the starting body temperature (mean (±SEM)) of pretreated male mice prior to onset of temperature induced seizures. The average body temperature of the male mice ranged from about 34.5° C. to about 35.5° C. prior to the inducement of the heat induced seizure.

FIG. 3 depicts the average starting body temperature (mean (±SEM)) of all pretreated mice prior to onset of temperature induced seizures. The average body temperature (mean (±SEM)) of the mice ranged from about 34.5° C. to about 35.3° C. prior to the inducement of the heat induced seizure.

FIG. 4 depicts the final body temperature (mean (±SEM)) of female mice in the seizure study. The final body temperature was increased to approximately 42° C. (ranging from about 40.5° C. to about 42.5° C.), which is sufficient to induce heat-induced seizures in the mouse model absent treatment with either clobazam or a GALBA_{A} receptor modulator.

FIG. 5 depicts the final body temperature (mean (±SEM)) of male mice in the seizure study. The final body temperature was increased to approximately 42° C. (ranging from about 40.5° C. to about 42.5° C.), which is sufficient to induce heat-induced seizures in the mouse model absent anti-epileptic treatment.

FIG. 6 depicts the average final body temperature (mean (±SEM)) of all mice in the seizure study. The final body temperature was increased to approximately 42° C. (ranging from about 40.5° C. to about 42.5° C.), which is sufficient to induce heat-induced seizures in the mouse model absent anti-epileptic treatment. Administration of the vehicle resulted in the lowest overall body temperature for the mice.

FIG. 7 depicts the total change in body temperature (mean (±SEM)) of female mice during the seizure study. The body temperature of the female mice increased between 5° C. and 8° C. in order to induce the heat-induced seizures in the mouse model.

FIG. 8 depicts the total change in body temperature (mean (±SEM)) of male mice during the seizure study. The body temperature of the male mice increased between 5° C. and 8° C. in order to induce the heat-induced seizures in the mouse model.

FIG. 9 depicts the average total change in body temperature (mean (±SEM)) of all mice during the seizure study. The body temperature of the mice increased between 5° C. and 8° C. in order to induce the heat-induced seizures in the mouse model. Mice administered the vehicle control had an overall temperature increase of 5° C., which was lower than the mice that received the other treatments.

FIG. 10 depicts the change in body temperature per minute (mean (±SEM)) of female mice during the seizure study. The change in body temperature per minute for the female mice ranged from about 0.4° C./min to about 0.6° C./min.

FIG. 11 depicts the change in body temperature per minute (mean (±SEM)) of male mice during the seizure study. The change in body temperature per minute for the male mice ranged from about 0.4° C./min to about 0.6° C./min.

FIG. 12 depicts the average change in body temperature per minute (mean (±SEM)) of all mice during the seizure study. The change in body temperature per minute for the mice ranged from about 0.4° C./min to about 0.6° C./min. Mice administered the vehicle had the highest rate of temperature increase per minute (about 0.6° C./min), while mice administered 100 mg/k of the exemplary GABA_{A} receptor modulator Compound 2 had the lowest rate of temperature increase per minute (about 0.4° C./min).

FIG. 13 depicts a summary of the efficacy of each treatment in the seizure study. The exemplary GABA_{A} receptor modulators Compound 1 and Compound 2 each were effective at preventing heat-induced seizures in the mouse model. Administration of the GABA_{A} receptor modulator Compound 2 prevented seizures in a dose-dependent fashion.

FIG. 14 depicts exemplary GABA_{A} receptor modulators that are effective to treat an epileptic condition.

### DETAILED DESCRIPTION

### Definitions

The term “GABA receptor” can be used in its meaning known in the field of biochemistry; it can refer to receptors of the inhibitory neurotransmitter gamma-aminobutyric acid (GABA). GABA_{A} receptors (ionotropic receptors) are ligand-gated ion channels and GABA_{B} receptors, also known as metabotropic receptors, are G protein-coupled receptors. GABA_{A} receptors are the most common and most important inhibitory receptors within the central nervous system. GABA_{A} receptors comprise five subunits that are grouped in eight classes: α₁₋₆, β₁₋₃, γ₁₋₃, δ, ε, π, θ and ρ₁₋₃. The majority of GABA_{A} receptors comprise two α, two β and one γ subunit. Compounds such as benzodiazepines can bind to sites distinct from the endogenous ligand GABA. For example, a benzodiazepine can bind to a binding site situated between the α and γ subunits. A compound described herein can be an allosteric modulator of a GABA_{A} receptor. The different types of a subunits confer different properties to the GABA_{A} receptor. Whereas the α1 subunit is among other functions responsible for the sedative effect of benzodiazepines, the α2 subunit is connected, among other things, to the anxiolytic function of the receptors and the α3 subunit confers, among other things, the muscle relaxing properties of the GABA_{A} receptor.

The term “allosteric modulator” can be used in its meaning known in the field of biochemistry and pharmacology; it can refer to a substance that indirectly modulates the effects of an agonist at a receptor. A positive allosteric modulator can induce an amplification of the agonist's effect, without having an effect by itself in the absence of the agonist. An allosteric modulator can bind to a site distinct from the agonist's binding site (allosteric).

The term “subject”, “patient” or “individual” as used herein can encompass a mammal and a non-mammal. A mammal can be any member of the Mammalian class, including but not limited to a human, a non-human primates such as a chimpanzee, an ape or other monkey species; a farm animal such as cattle, a horse, a sheep, a goat, a swine; a domestic animal such as a rabbit, a dog (or a canine), and a cat (or a feline); a laboratory animal including a rodent, such as a rat, a mouse and a guinea pig, and the like. A non-mammal can include a bird, a fish and the like. In some embodiments, a subject can be a mammal. In some embodiments, a subject can be a human. In some instances, a human can be an adult. In some instances, a human can be a child. In some instances, a human can be age 0-17 years old. In some instances, a human can be age 18-130 years old. In some instances, a subject can be a male. In some instances, a subject can be a female. In some instances, a subject can be diagnosed with, or can be suspected of having, a condition or disease. In some instances, a disease or condition, or an epileptic condition. A subject can be a patient. A subject can be an individual. In some instances, a subject, patient or individual can be used interchangeably.

The terms “treat,” “treating”, “treatment,” “ameliorate” or “ameliorating” and other grammatical equivalents as used herein, can include alleviating, or abating a disease or condition symptoms, inhibiting a disease or condition, e.g., arresting the development of a disease or condition, relieving a disease or condition, causing regression of a disease or condition, relieving a condition caused by the disease or condition, or stopping symptoms of a disease or condition.

An “alkyl” can refer to a saturated linear or branched hydrocarbon having e.g., 1, 2, 3, 4, 5, 6, 7, 8, 9, 10, 11, 12 or more carbon atoms, wherein one carbon-carbon bond may be unsaturated and one CH₂ moiety may be exchanged for oxygen (ether bridge). Non-limiting examples for a C₁-C₄ alkyl are methyl, ethyl, propyl, prop-2-enyl, n-butyl, 2-methylpropyl, tert-butyl, but-3-enyl, prop-2-inyl and but-3-inyl. Unless stated otherwise specifically in the specification, an alkyl group may be optionally substituted.

The term “Alkynyl” can refer to an optionally substituted straight-chain or optionally substituted branched-chain hydrocarbon monoradical having one or more carbon-carbon triple-bonds. In some embodiments, an alkynyl group can have from two to about ten carbon atoms, more preferably from two to about six carbon atoms. Examples can include, but not limited to, ethynyl, 2-propynyl, 2-butynyl, 1,3-butadiynyl, and the like. Whenever it appears herein, a numerical range such as “C₂-C₆ alkynyl” can mean that the alkynyl group may consist of 2 carbon atoms, 3 carbon atoms, 4 carbon atoms, 5 carbon atoms, or 6 carbon atoms, although the present definition can also cover the occurrence of the term “alkynyl” where no numerical range can be designated. In some embodiments, the alkynyl can be a C₁-C₁₀ alkynyl, a C₁-C₉ alkynyl, a C₁-C₈ alkynyl, a C₁-C₇ alkynyl, a C₁-C₆ alkynyl, a C₁-C₅ alkynyl, a C₁-C₄ alkynyl, a C₁-C₃ alkynyl, a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group can be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl can be optionally substituted with oxo, halogen, —CN, —CF₃, —OH, —OMe, —NH₂, or —NO₂. In some embodiments, an alkynyl can be optionally substituted with oxo, halogen, —CN, —CF₃, —OH, or —OMe. In some embodiments, the alkynyl can be optionally substituted with halogen.

The term “aryl” can refer to a cyclic aromatic C₅-C₁₀ hydrocarbon. Examples of aryl can include, without being restricted to, phenyl, naphthyl and heteroaryl. The term “heteroaryl” can refer to aryl compounds in which at least one carbon atom is replaced with an oxygen, a nitrogen or a sulfur atom. Examples for heteroaryl can include, without being restricted to, pyrrole, thiophene, furan, imidazole, pyrazole, thiazole, oxazole, pyridine, pyrimidine, thiazin, quinoline, benzofuran and indole. An aryl or a heteroaryl can be optionally substituted. The aromatic hydrocarbon may be neutral or charged. Aryl or heteroaryl groups as used herein may optionally include one or more further substituent groups.

“Cycloalkyl” can refer to a stable, partially or fully saturated, monocyclic or polycyclic carbocyclic ring, which can include fused (when fused with an aryl or a heteroaryl ring, the cycloalkyl can be bonded through a non-aromatic ring atom), bridged, or spiro ring systems. Representative cycloalkyls can include, but not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl can be a 3- to 6-membered cycloalkyl. In some embodiments, the cycloalkyl can be a 5- to 6-membered cycloalkyl. Monocyclic cycloalkyls can include, for example, cyclopropyl, cyclobutyl, cyclopentyl, cyclohexyl, cycloheptyl, and cyclooctyl. Polycyclic cycloalkyls or carbocycles can include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls can include, for example, cyclopentenyl, cyclohexenyl, cycloheptenyl, and cyclooctenyl. Unless stated otherwise specifically in the specification, a cycloalkyl can be optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl can be optionally substituted with oxo, halogen, methyl, ethyl, —CN, —CF₃, —OH, —OMe, —NH₂, or —NO₂. In some embodiments, a cycloalkyl can be optionally substituted with oxo, halogen, methyl, ethyl, —CN, —CF₃, —OH, or —OMe. In some embodiments, the cycloalkyl is optionally substituted with halogen.

“Halo” or “halogen” can refer to bromo (Br), chloro (Cl), fluoro (F), or iodo (I). In some embodiments, halogen can be Br, F or Cl. In some embodiments, halogen can be F.

The term “about” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

Conditions

Disclosed herein are compounds for treatment of epileptic conditions. Compounds described herein (e.g. GABA_{A} receptor modulators) can be used to at least partially ameliorate epileptic condition. For example, administration of a GABA_{A} receptor modulator can effectively treat onset of seizures associated with an epileptic condition. The term epileptic condition as used herein can refer to a condition associated with seizure. In some embodiments, the treatment comprises reducing or preventing an onset of seizures associated with the epileptic condition. In some embodiments, a seizure is accompanied by convulsion. In some embodiments, an epileptic condition with seizures is also accompanied by convulsions. In some embodiments, an epileptic condition with seizures may not be accompanied by convulsions.

Seizures are often associated with a number of diseases or conditions. For example, seizure can be associated with Angelman syndrome, Arteriovenous malformation, Brain abscess, Brain tumor, Cavernoma, Cerebral palsy, Down syndrome, Eclampsia, Epilepsy, Encephalitis, Fragile X syndrome, Meningitis, Multiple sclerosis, Systemic lupus erythematosus, and Tuberous sclerosis. Further, seizures can be associated with adverse effects of certain drugs, including Aminophylline, Bupivicaine, Bupropion, Butyrophenones, excessively high caffeine, Chlorambucil, Ciclosporin, Clozapine, Corticosteroids, Diphenhydramine, Enflurane, Estrogens, Fentanyl, Insulin, Lidocaine, Maprotiline, Meperidine, Olanzapine, Pentazocine, Phenothiazines, Prednisone, Procaine, Propofol, Propoxyphene, Quetiapine, Risperidone, Sevoflurane, Theophylline, Tramadol, Tricyclic antidepressants, Venlafaxine, isoniazid, lindane, metronidazole, nalidixic acid, penicillin, fluoroquinolones, and carbapenems.

In some cases, the epileptic condition is epilepsy. Epilepsy is a common chronic neurological disorder that is characterized by recurrent unprovoked seizures. These seizures are transient signs and/or symptoms due to abnormal, excessive or synchronous neuronal activity in the brain. There are many different epileptic conditions, each presenting with its own unique combination of seizure type, typical age of onset, EEG findings, treatment, and prognosis. In some embodiments, the epileptic condition is a general epilepsy. In some embodiments, the epileptic condition is genetic epilepsy.

Other exemplary epileptic conditions include those conditions that can occur with seizures, e.g., Benign centrotemporal lobe epilepsy of childhood, Benign occipital epilepsy of childhood (BOEC), Autosomal dominant nocturnal frontal lobe epilepsy (ADNFLE), Primary reading epilepsy, Childhood absence epilepsy (CEA), Juvenile absence epilepsy, Juvenile myoclonic epilepsy (JME), Symptomatic localization-related epilepsies, Temporal lobe epilepsy (TLE), Frontal lobe epilepsy, Rasmussen's encephalitis, Cerebral palsy, Cerebral hypoxia, Down's syndrome, hypoxic-ischemic encephalopathy (HIE), West syndrome, Dravet syndrome, Progressive myoclonic epilepsies, and Lennox-Gastaut syndrome (LGS). Genetic, congenital, and developmental conditions are often associated with epilepsy among younger patients. Tumors might be a cause for patients over age 40. Head trauma and central nervous system infections may cause epilepsy at any age. In some cases, a GABAA receptor modulator can be administered to treat a breakthrough seizure. A “breakthrough seizure” as described herein can refer to a seizure that occurs after a prolonged time period of seizure freedom. In some cases, the breakthrough seizure can occur due to a subject not taking a medicine. In some cases, the breakthrough seizure can occur due to a tolerance or recalcitrance of the seizure to existing therapeutics such as clobazam. Accordingly, a GABA_{A} receptor modulator as described herein can be used as a replacement first line treatment to treat breakthrough seizures.

In some embodiments, the epileptic condition is a focal seizure. The focal seizure can be a simple focal seizure (aura). A simple focal seizure with motor symptoms can affect muscle activity, causing jerking movements of a foot, the face, an arm or another part of the body. A simple focal seizure may cause sensory symptoms affecting the senses, such as hearing problems, hallucinations and olfactory or other distortions. A simple focal seizure with autonomic symptoms can affect the part of the brain responsible for involuntary functions. These seizures can cause changes in blood pressure, heart rhythm, or bowel or bladder function. Some simple focal seizures can strike parts of the brain that trigger emotions or memories of previous experiences, causing feelings of fear, anxiety, or the illusory feeling that something has been experienced before. The focal seizure can be a complex focal seizure. In some cases, complex focal seizures are preceded by a simple focal seizure. In some cases, subjects experiencing a complex focal seizure may stare blankly into space, or experience automatisms (non-purposeful, repetitive movements such as lip smacking, blinking, grunting, gulping or shouting).

In some cases, a disease or condition treatable by a GABAA receptor modulator as described herein is an epilepsy associated with a mutation in a sodium channel in a subject. Such a disease or condition treatable by a GABA_{A} receptor modulator as described herein is Dravet syndrome. Dravet syndrome is a rare genetic epileptic encephalopathy. In some cases, Dravet Syndrome is associated with a mutation in a sodium channel. For example, Dravet syndrome can be associated with a mutation in a sodium voltage-gated channel alpha subunit 1 (SCN1A) gene. Such a mutation in SCN1A can include a missense mutation, a nonsense mutation, a frameshift mutation, a splice mutation, or an in-frame deletion. For example, an SCN1A mutation can comprise an D79H, R101Q, R222X, I227S, R377X, R393C, R613X, R712X, R859C, R1596C, R1213X, R1648H, M1780T, A1783V, R1892X, or R1912X mutation.

Dravet syndrome may begin in infancy and persist through the lifetime of a subject. Dravet syndrome can manifest as different seizure types, including myoclonic seizure, tonic-clonic seizure, absence seizure, atypical absence seizure, atonic seizure, focal aware seizure, or status epilepticus. A GABA_{A} receptor modulator as described herein can be administered to a subject suffering from Dravet syndrome to at least partially ameliorate seizures associated with Dravet syndrome.

As described in the examples in the present application, GABA_{A} receptor modulators are effective at treating hyperthermia-induced seizures in a Scn1a+/− knockout mouse model. This Scn1a^{+/-} knockout mouse model is recognized in the art as a model for various epileptic conditions, including Dravet Syndrome. Thus, GABA_{A} receptor modulators as a class are expected to have efficacy at treating or preventing seizures in various epileptic conditions as described herein.

Compounds

Disclosed herein are compounds for treatment of epileptic conditions. In some cases, a compound for treatment of epileptic conditions can be a GABA_{A} receptor modulator. In certain embodiments, the compound is a positive allosteric α2 and/or α3 GABA_{A} receptor modulator. A GABA_{A} receptor modulator that is specific for an α2 or an α3 subunit, while avoiding modulation of an α1 subunit may be useful for treating epileptic conditions as described herein without causing drowsiness associated with α1 subunit modulation. Accordingly, a GABA_{A} receptor modulator as described herein can be formulated as a non-sedating or non-drowsy formulation. In some cases, a non-drowsy formulation can contain other components including stimulants (e.g. caffeine) that can further counteract any sedative effects associated with the administering.

A GABAA receptor modulator, and metabolites, pharmaceutically acceptable salts, esters, prodrugs, solvate, hydrates or derivatives thereof can be included in exemplary embodiments, and are expected to be effective at treating an epileptic condition as described herein.

A GABA_{A} receptor modulator that can be used in the treatment of epileptic conditions can be a compound of general formula (1a), (1b), or (1c):

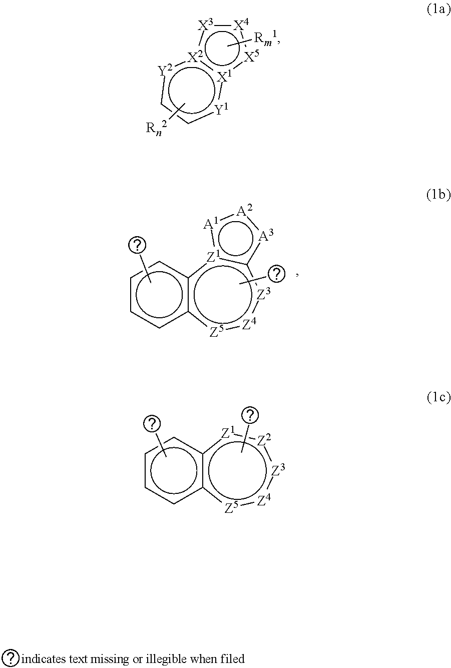

where
X¹, X², X³, X⁴ and X⁵ are independently from each other —C, —N, —S or —O wherein at least two of X¹, X², X³, X⁴ and X⁵ are —N,
Y¹ and Y² are independently from each other —C or —N,
m of R¹ₘ is 1,
R¹ is a substituted or unsubstituted C₆ aryl or —(C═O)—R³, with R³ being a substituted or unsubstituted C₆ heteroaryl,
n of R²ₙ is 1 or 2,
each R² independently from any other R² is a substituted or unsubstituted C₃-C₈ cycloalkyl, a substituted or unsubstituted C₁-C₆ alkyl, a substituted or unsubstituted C₁-C₆ alcohol, a substituted or unsubstituted C₆ heteroaryl, a halogen, in some instances —F, or —O—CH₂—R⁴, with R⁴ being a substituted or unsubstituted C₄ heteroaryl,
Z¹, Z², Z³, Z⁴ and Z⁵ are independently of each other —C, —N, —S or —O,
A¹, and A² and A³, are independently of each other —C, —N, —(C═O)—O—R⁷, or

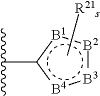

where R⁷ is alkyl,
B¹, B², B³, and B⁴ are independently of each other —C, —N, or —O,
s of R²¹ₛ is 1, 2, 3 or 4, and
each R²¹ is independently hydrogen or C₁-C₆ alkyl,
l of R⁵ₗ is 1 or 2,
each R⁵ is independently from each other a C₁-C₄ alkynyl or a halogen, in some instances —Cl,
k of R⁶ₖ is 1, 2, 3 or 4, in some instances 1 or 2,
each R⁶ is independently from each other a substituted or unsubstituted aryl, a substituted or unsubstituted heteroaryl, a substituted or unsubstituted C₁-C₃ alkyl, oxygen or hydrogen.

In certain embodiments a compound comprising the general formula (1a) is provided, wherein X¹, X², X³, X⁴ and X⁵ are independently from each other —C, —N, —S or —O, wherein at least two of X¹, X², X³, X⁴ and X⁵ are —N, Y¹ and Y² are independently from each other —C or —N, m of R¹ₘ is 1, R¹ is a substituted or unsubstituted C₆ aryl or —(C═O)—R³, with R³ being a substituted or unsubstituted C₆ heteroaryl, n of R²ₙ is 1 or 2, each R² independently from any other R² is a substituted or unsubstituted C₃-C₈ cycloalkyl, a substituted or unsubstituted C₁-C₆ alkyl, a substituted or unsubstituted C₁-C₆ alcohol, a substituted or unsubstituted C₆ heteroaryl, a halogen, in some instances —F, or —O—CH₂—R⁴, with R⁴ being a substituted or unsubstituted C₄ heteroaryl.

In certain embodiments, a compound comprising the general formula (1b) is provided, wherein Z³, Z⁴ and Z⁵ are independently of each other —C, —N, —S or —O, A¹, A² and A³, are independently of each other —C, —N or —(C═O)—O—R⁷, with R⁷ being an alkyl, l of R⁵ₗ is 1 or 2, each R⁵ is independently from each other a C₁-C₄ alkynyl or a halogen, in some instances —Cl, k of R⁶ₖ is 1, 2, 3 or 4, in some instances 1 or 2, each R⁶ is independently from each other a substituted or unsubstituted aryl, a substituted or unsubstituted heteroaryl, a substituted or unsubstituted C₁-C₃ alkyl, oxygen or hydrogen.

In certain embodiments, a compound comprising the general formula (1c) is provided, wherein Z¹, Z², Z³, Z⁴ and Z⁵ are independently of each other —C, —N, —S or —O, l of R⁵ is 1 or 2, each R⁵ is independently from each other a C₁-C₄ alkynyl or a halogen, in some instances —Cl, k of R⁶ₖ is 1, 2, 3 or 4, in some instances 1 or 2, each R⁶ is independently from each other a substituted or unsubstituted aryl, a substituted or unsubstituted heteroaryl, a substituted or unsubstituted C₁-C₃ alkyl, oxygen or hydrogen.

In certain embodiments, the compound comprises the general formula (1a), general formula (1b) or general formula (1c), wherein X¹, X², X³, X⁴ and X⁵ are independently from each other —C, —N, wherein at least two of X¹, X², X³, X⁴ and X⁵ are —N, Y¹ and Y² are independently from each other —C or —N, m of R¹ₘ is 1, R¹ is a substituted or unsubstituted C₆ aryl or —(C═O)—R³, with R³ being a substituted or unsubstituted C₆ heteroaryl, n of R²ₙ is 1 or 2, each R² independently from any other R² is a substituted or unsubstituted C₃-C₈ cycloalkyl, a substituted or unsubstituted C₁-C₆ alkyl, a substituted or unsubstituted C₁-C₆ alcohol, a substituted or unsubstituted C₆ heteroaryl, a halogen, in some instances —F, or —O—CH₂—R⁴, with R⁴ being a substituted or unsubstituted C₄ heteroaryl, Z¹, Z², Z³, Z⁴ and Z⁵ are independently of each other —C, —N or —O, A¹, A² and A³, are independently of each other —C, —N or —(C═O)—O—R⁷, with R⁷ being an alkyl, l of R⁵ₗ is 1 or 2, each R⁵ is independently from each other a C₁-C₄ alkynyl or a halogen, in some instances —Cl, k of R⁶ₖ is 1, 2, 3 or 4, each R⁶ is independently from each other a substituted or unsubstituted aryl, a substituted or unsubstituted heteroaryl, a substituted or unsubstituted C₁-C₃ alkyl, oxygen or hydrogen.

In certain embodiments a compound comprising the general formula (1a) is provided, wherein X¹, X², X³, X⁴ and X⁵ are independently from each other —C or —N, wherein at least two of X¹, X², X³, X⁴ and X⁵ are —N, Y¹ and Y² are independently from each other —C or —N, m of R¹ₘ is 1, R¹ is a substituted or unsubstituted C₆ aryl or —(C═O)—R³, with R³ being a substituted or unsubstituted C₆ heteroaryl, n of R²ₙ is 1 or 2, each R² independently from any other R² is a substituted or unsubstituted C₃-C₈ cycloalkyl, a substituted or unsubstituted C₁-C₆ alkyl, a substituted or unsubstituted C₁-C₆ alcohol, a substituted or unsubstituted C₆ heteroaryl, a halogen, in some instances —F, or —O—CH₂—R⁴, with R⁴ being a substituted or unsubstituted C₄ heteroaryl.

In certain embodiments, a compound comprising the general formula (1b) is provided, wherein Z³, Z⁴ and Z⁵ are independently of each other —C or —N, A¹, A² and A³, are independently of each other —C, —N or —(C═O)—O—R⁷, with R⁷ being an alkyl, l of R⁵ₗ is 1 or 2, each R⁵ is independently from each other a C₁-C₄ alkynyl or a halogen, in some instances —Cl, k of R⁶ₖ is 1, 2, 3 or 4, in some instances 1 or 2, each R⁶ is independently from each other a substituted or unsubstituted aryl, a substituted or unsubstituted heteroaryl, a substituted or unsubstituted C₁-C₃ alkyl, oxygen or hydrogen.

In certain embodiments, a compound comprising the general formula (1c) is provided, wherein Z¹, Z², Z³, Z⁴ and Z⁵ are independently of each other —C, —N or —O, I of R⁵ is 1 or 2, each R⁵ is independently from each other a C₁-C₄ alkynyl or a halogen, in some instances —Cl, k of R⁶ₖ is 1, 2, 3 or 4, in some instances 1 or 2, each R⁶ is independently from each other a substituted or unsubstituted aryl, a substituted or unsubstituted heteroaryl, a substituted or unsubstituted C₁-C₃ alkyl, oxygen or hydrogen.

A GABA_{A} receptor modulator that can be used in the treatment of epileptic conditions can be a compound of general formula (2), (3), (4), (5), (6) or (7)

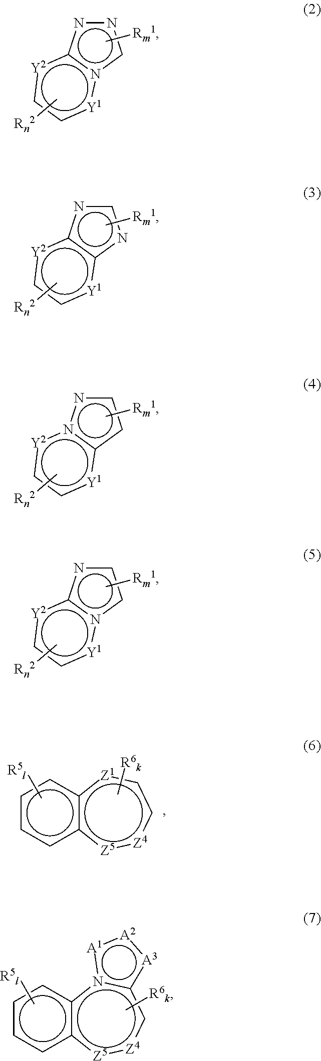

with Y¹, Y², Z¹, Z⁴, Z⁵, m of R¹ₘ, R¹, R³, n of R²ₙ, R², R⁴, l of R⁵ₗ, R⁵, k of R⁶ₖ, R⁶, A¹, A² and A³ having the same meaning as defined above.

In certain embodiments, the compound comprises the general formula (2), (3), (4) or (5), with Y¹, Y², m of R¹ₘ, R¹, R³, n of R²ₙ, R² and R⁴ having the same meaning as defined above.

In certain embodiments, the compound comprises the general formula (6) with Z¹, Z⁴, Z⁵, l of R⁵ₗ, R⁵, k of R⁶ₖ and R⁶ having the same meaning as defined above.

In certain embodiments, the compound comprises the general formula (7) with Z⁴, Z⁵, l of R⁵ₗ, R⁵, k of R⁶ₖ, R⁶, A¹, A² and A³ having the same meaning as defined above.

A GABA_{A} receptor modulator that can be used in the treatment of epileptic conditions can be a compound of general formula (2a), (3a), (4a), (5a), (5b), (6a), (6b) or (7a)

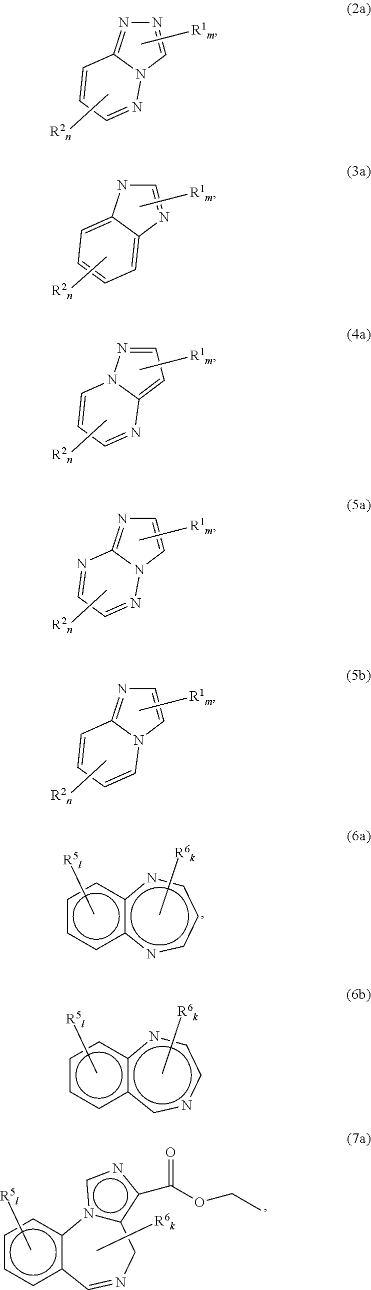

with m of R¹ₘ, R¹, R³, n of R²ₙ, R², R⁴, l of R⁵ₗ R⁵, k of R⁶ₖ and R⁶ having the same meaning as defined above.

In certain embodiments, the compound comprises the general formula (2a), (3a), (4a), (5a) or (5b) with m of R¹ₘ, R¹, R³, n of R²ₙ, R² and R⁴ having the same meaning as defined above.

In certain embodiments, the compound comprises the general formula (6a) or (6b) with m of l of R⁵ₗ, R⁵, k of R⁶ₖ and R⁶ having the same meaning as defined above.

In certain embodiments, the compound comprises the general formula (7a) with m of 1 of R⁵ₗ, R⁵, k of R⁶ₖ and R⁶ having the same meaning as defined above.

In certain embodiments, the compound comprises the general formula 1a, 1b, 1c, 2, 3, 4, 5, 6, 7, 2a, 3a, 4a, 5a, 5b, 6a, 6b or 7a, wherein m of R¹ is l and R¹ is: an unsubstituted phenyl, a substituted phenyl comprising at least one —F as a substituent, an unsubstituted biphenyl, a substituted biphenyl comprising at least one —CN as a substituent, in some instances on the phenyl moiety not connected to the parent moiety, or a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, or —(C═O)—R³, with R³ being pyridine, and I of R⁵ is 1 and R⁵ is Cl, Br, F, or a C₂ alkynyl.

In certain embodiments, the compound comprises the general formula 1a, 2, 3, 4, 5, 2a, 3a, 4a, 5a or 5b, wherein m of R¹ is 1 and R¹ is: an unsubstituted phenyl, a substituted phenyl comprising at least one —F as a substituent, an unsubstituted biphenyl, a substituted biphenyl comprising at least one —CN as a substituent, in some instances on the phenyl moiety not connected to the parent moiety, or a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, or —(C═O)—R³, with R³ being pyridine.

In certain embodiments, the compound comprises the general formula 1b, 7, or 7a, wherein l of R⁵ is 1 and R⁵ is Cl, Br, F, or a C₂ alkynyl.

In certain embodiments, the compound comprises the general formula 1c, 6, 6a or 6b, wherein l of R⁵ is 1 and R⁵ is Cl, Br, F, or a C₂ alkynyl.

In certain embodiments, the compound comprises the general formula 1a, 1b, 1c, 2, 3, 4, 5, 6, 7, 2a, 3a, 4a, 5s, 5b, 6a, 6b or 7a, wherein n of R² is 1 or 2 and in case of n being 2, each R² independently from each other is an unsubstituted C₃-C₈ cycloalkyl, in some instances a C₄-cycloalkyl, an unsubstituted C₁-C₆ alkyl, in some instances tert-butyl, or —O—CH₂—R⁴, with R⁴ being a substituted or unsubstituted C₄ heteroaryl, in some instances R⁴ being a substituted or unsubstituted triazole, an unsubstituted C₁-C₆ alcohol, in some instances a C₄ alcohol, a halogen, in some instances —F, in case of n being 1, R² is an unsubstituted C₁-C₆ alcohol, in some instances a C₄ alcohol, an unsubstituted C₆ heteroaryl, in some instances pyridine, and k of R⁶ is 1 or 4 and in case of k being 1, R⁶ is a substituted or unsubstituted aryl, a substituted or unsubstituted heteroaryl, in case of k being 4, each R⁶ independently from each other is a substituted or unsubstituted aryl, a substituted or unsubstituted C₁-C₃ alkyl, oxygen, or hydrogen.

In certain embodiments, the compound comprises the general formula 1a, 1b, 1c, 2, 3, 4, 5, 6, 7, 2a, 3a, 4a, 5a, 5b, 6a, 6b or 7a, wherein n of R² is 1 or 2 and in case of n being 2, each R² independently from each other is an unsubstituted C₃-C₈ cycloalkyl, in some instances a C₄-cycloalkyl, an unsubstituted C₁-C₆ alkyl, in some instances tert-butyl, or —O—CH₂—R⁴, with R⁴ being a substituted or unsubstituted C₄ heteroaryl, in some instances R⁴ being a substituted or unsubstituted triazole, an unsubstituted C₁-C₆ alcohol, in some instances a C₄ alcohol, a halogen, in some instances —F, in case of n being 1, R² is an unsubstituted C₁-C₆ alcohol, in some instances a C₄ alcohol, an unsubstituted C₆ heteroaryl, in some instances pyridine, and k of R⁶ is 1 or 4 and in case of k being 1, R⁶ is a substituted or unsubstituted aryl, a substituted or unsubstituted heteroaryl, in case of k being 4, each R⁶ independently from each other is a substituted or unsubstituted aryl, a substituted or unsubstituted C₁-C₃ alkyl, oxygen, or hydrogen.

In certain embodiments, the compound comprises the general formula 1a, 2, 3, 4, 5, 2a, 3a, 4a, 5a or 5b, wherein n of R² is 1 or 2 and in case of n being 2, each R² independently from each other is an unsubstituted C₃-C₈ cycloalkyl, in some instances a C₄-cycloalkyl, an unsubstituted C₁-C₆ alkyl, in some instances tert-butyl, or —O—CH₂—R⁴, with R⁴ being a substituted or unsubstituted C₄ heteroaryl, in some instances R⁴ being a substituted or unsubstituted triazole, an unsubstituted C₁-C₆ alcohol, in some instances a C₄ alcohol, a halogen, in some instances —F, in case of n being 1, R² is an unsubstituted C₁-C₆ alcohol, in some instances a C₄ alcohol, an unsubstituted C₆ heteroaryl, in some instances pyridine.

In certain embodiments, the compound comprises the general formula 1b, 7, or 7a, wherein k of R⁶ is 1 or 4 and in case of k being 1, R⁶ is a substituted or unsubstituted aryl, a substituted or unsubstituted heteroaryl, in case of k being 4, each R⁶ independently from each other is a substituted or unsubstituted aryl, a substituted or unsubstituted C₁-C₃ alkyl, oxygen, or hydrogen.

In certain embodiments, the compound comprises the general formula 1c, 6, 6a or 6b wherein k of R⁶ is 1 or 4 and in case of k being 1, R⁶ is a substituted or unsubstituted aryl, a substituted or unsubstituted heteroaryl, in case of k being 4, each R⁶ independently from each other is a substituted or unsubstituted aryl, a substituted or unsubstituted C₁-C₃ alkyl, oxygen, or hydrogen.

In certain embodiments, the compound comprises the general formula 1a, 1b, 1c, 2, 3, 4, 5, 6, 7, 2a, 3a, 4a, 5s, 5b, 6a, 6b or 7a, wherein n of R² is 2 and one R² is an unsubstituted C₃-C₈ cycloalkyl, in some instances a C₄-cycloalkyl, or an unsubstituted C₁-C₆ alkyl, in some instances tert-butyl, and the other R² is —O—CH₂—R⁴, with R⁴ being a substituted or unsubstituted C₄ heteroaryl, in some instances a substituted or unsubstituted triazole, or one R² is an unsubstituted C₁-C₆ alcohol, in some instances a C₄ alcohol, and the other R² is a halogen, in some instances —F, and k of R⁶ is 4 and two R⁶ are oxygen, the other R⁶ are independently from each other a substituted or unsubstituted aryl, a substituted or unsubstituted C₁-C₃ alkyl, or hydrogen.

In certain embodiments, the compound comprises the general formula 1a, 2, 3, 4, 5, 2a, 3a, 4a, 5s or 5b, wherein n of R² is 2 and one R² is an unsubstituted C₃-C₈ cycloalkyl, in some instances a C₄-cycloalkyl, or an unsubstituted C₁-C₆ alkyl, in some instances tert-butyl, and the other R² is O—CH₂—R⁴, with R⁴ being a substituted or unsubstituted C₄ heteroaryl, in some instances a substituted or unsubstituted triazole, or one R² is an unsubstituted C₁-C₆ alcohol, in some instances a C₄ alcohol, and the other R² is a halogen, in some instances —F.

In certain embodiments, the compound comprises the general formula 1b, 7, or 7a, wherein k of R⁶ is 4 and two R⁶ are oxygen, the other R⁶ are independently from each other a substituted or unsubstituted aryl, a substituted or unsubstituted C₁-C₃ alkyl, or hydrogen.

In certain embodiments, the compound comprises the general formula 1c, 6, 6a or 6b, wherein k of R⁶ is 4 and two R⁶ are oxygen, the other R⁶ are independently from each other a substituted or unsubstituted aryl, a substituted or unsubstituted C₁-C₃ alkyl, or hydrogen.

A GABA_{A} receptor modulator that can be used in the treatment of epileptic conditions can be a compound of general formula (2a′), (3a′), (4a′), (5a′), (5b′), (6a′), (6b′) or (7a′)

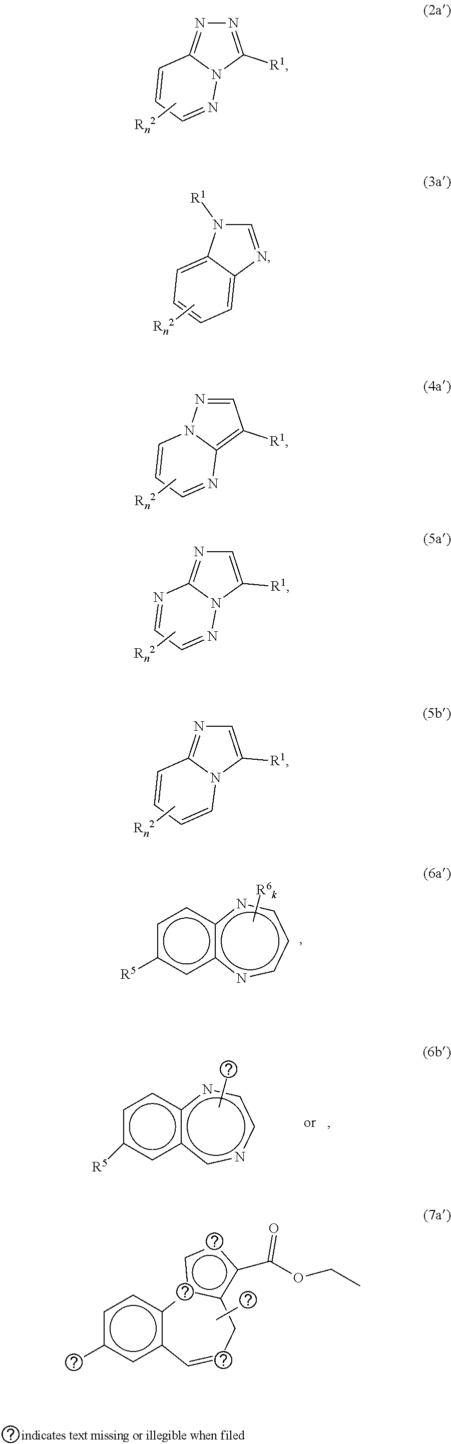

with R¹, R³ₙ of R²ₙ, R², R⁴, R⁵, k of R⁶ₖ and R⁶ having the same meaning as defined above.

In certain embodiments, the compound comprises the general formula (2a′), (3a′), (4a′), (5a′) or (5b′) with R¹, R³, n of R², R² and R⁴ having the same meaning as defined above.

In certain embodiments, the compound comprises the general formula (6a′) or (6b′) with k of R⁶ₖ and R⁶ having the same meaning as defined above.

In certain embodiments, the compound comprises the general formula (7a′) with k of R⁶ₖ and R⁶ having the same meaning as defined above.

In certain embodiments, the compound comprises the general formula (2a′), (3a′), (4a′), (5a′), (5b′), (6a′), (6b′) or (7a′), wherein R^{r} is in case of formula (2a′) a substituted or unsubstituted C₆ aryl, in some instances an unsubstituted phenyl, a substituted phenyl comprising at least one —F as a substituent, in case of formula (3a′), (5a′) or (5b′), a substituted or unsubstituted biphenyl, in some instances an unsubstituted biphenyl, a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, or in case of formula (4a′), —(C═O)—R³, with R³ being a substituted or unsubstituted C₆ heteroaryl, in some instances with R³ being pyridine, R⁵ is in case of formula (6a′) or (6b′), Cl, Br or F, in case of formula (7a′) C₂ alkynyl, wherein R²ₙ and R⁶ₖ have the same meaning as defined above.

In certain embodiments, the compound comprises the general formula (2a′), (3a′), (4a′), (5a′) or (5b′) wherein R^{r} is in case of formula (2a′) a substituted or unsubstituted C₆ aryl, in some instances an unsubstituted phenyl, a substituted phenyl comprising at least one —F as a substituent, in case of formula (3a′), (5a′) or (5b′), a substituted or unsubstituted biphenyl, in some instances an unsubstituted biphenyl, a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, or in case of formula (4a′), —(C═O)—R³, with R³ being a substituted or unsubstituted C₆ heteroaryl, in some instances with R³ being pyridine, wherein R²ₙ has the same meaning as defined above.

In certain embodiments, the compound comprises the general formula (6a′) or (6b′), wherein R⁵ is Cl, Br or F, wherein R⁶ₖ has the same meaning as defined above.

In certain embodiments, the compound comprises the general formula (7a′), wherein R⁵ is C² alkynyl, wherein R⁶ₖ has the same meaning as defined above.

A GABA_{A} receptor modulator that can be used in the treatment of epileptic conditions can be a compound of general formula (2a″), (3a″), (4a″), (5a″), (5b″), (6a″) or (7a″)

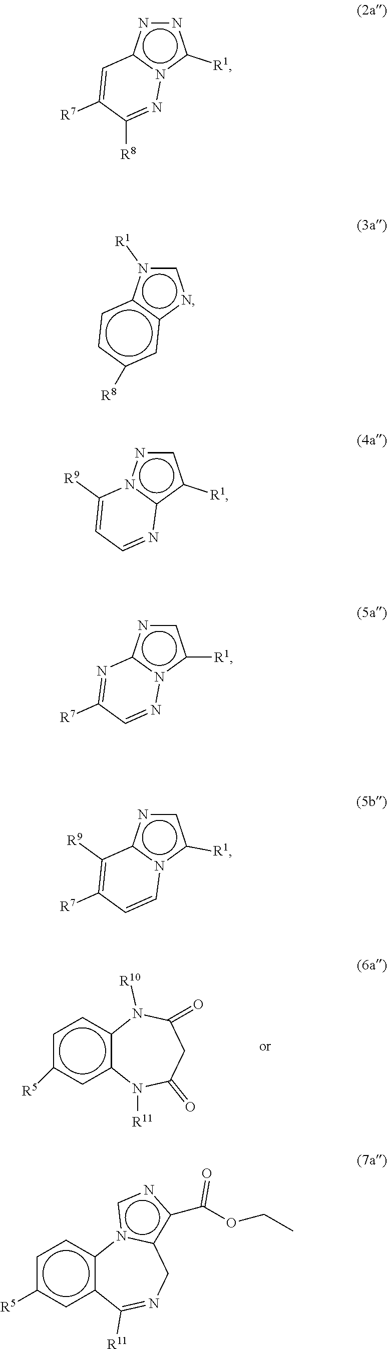

with R⁷ being an unsubstituted C₁-C₆ alkyl, in some instances tert-butyl, an unsubstituted C₃-C₈ cycloalkyl, in some instances a C₄-cycloalkyl, an unsubstituted C₁-C₆ alcohol, in some instances a C₄ alcohol, in some instances R⁷ being in case of formula (2a″) an unsubstituted C₁-C₆ alkyl, in some instances tert-butyl, or an unsubstituted C₃-C₈ cycloalkyl, in some instances a C₄-cycloalkyl, or R⁷ being in case of formula (5a″) or (5b″) an unsubstituted C₁-C₆ alcohol, in some instances a C₄ alcohol, R⁸ being —O—CH₂—R⁴, with R⁴ being a substituted or unsubstituted C₄ heteroaryl, in some instances a substituted or unsubstituted triazole, or an unsubstituted C₁-C₆ alcohol, in some instances a C₄ alcohol, in some instances R⁸ being in case of formula (2a″) —O—CH₂—R⁴, with R⁴ being a substituted or unsubstituted C₄ heteroaryl, in some instances a substituted or unsubstituted triazole, or R⁸ being in case of formula (3a″) an unsubstituted C₁-C₆ alcohol, in some instances a C₄ alcohol, R⁹ being an unsubstituted C₆ heteroaryl, in some instances pyridine, or a halogen, in some instances —F, R⁹ being in case of formula (4a″) an unsubstituted C₆ heteroaryl, in some instances pyridine, or R⁹ being in case of formula (5b″) a halogen, in some instances —F, R¹⁰ being a C₁-C₃ alkyl or hydrogen, R¹¹ being a substituted or unsubstituted aryl or a substituted or unsubstituted heteroaryl, in some instances R¹¹ being in case of formula (6a″) an substituted or unsubstituted aryl, in some instances phenyl R¹¹ being in case of formula (7a″) a substituted or unsubstituted heteroaryl, in some instances pyridine, a substituted or unsubstituted aryl, in some instances phenyl, R⁵ being in case of formula (6a″), Cl, Br or F, in case of formula (7a″) C₂ alkynyl.

In certain embodiments, the compound comprises the general formula (2a″), (3a″), (4a″), (5a″) or (5b″) with R⁷ being an unsubstituted C₁-C₆ alkyl, in some instances tert-butyl, an unsubstituted C₃-C₈ cycloalkyl, in some instances a C₄-cycloalkyl, an unsubstituted C₁-C₆ alcohol, in some instances a C₄ alcohol, in some instances R⁷ being in case of formula (2a″) an unsubstituted C₁-C₆-alkyl, in some instances tert-butyl, or an unsubstituted C₃-C₈ cycloalkyl, in some instances a C₄-cycloalkyl, or R⁷ being in case of formula (5a″) or (5b″) an unsubstituted C₁-C₆ alcohol, in some instances a C₄ alcohol, R⁹ being —O—CH₂—R⁴, with R⁴ being a substituted or unsubstituted C₄ heteroaryl, in some instances a substituted or unsubstituted triazole, or an unsubstituted C₁-C₆ alcohol, in some instances a C₄ alcohol, in some instances R⁹ being in case of formula (2a″) —O—CH₂—R⁴, with R⁴ being a substituted or unsubstituted C₄ heteroaryl, in some instances a substituted or unsubstituted triazole, or R⁸ being in case of formula (3a″) an unsubstituted C₁-C₆ alcohol, in some instances a C₄ alcohol, R⁹ being an unsubstituted C₆ heteroaryl, in some instances pyridine, or a halogen, in some instances —F, R⁹ being in case of formula (4a″) an unsubstituted C₆ heteroaryl, in some instances pyridine, or R⁹ being in case of formula (5b″) a halogen, in some instances —F.

In certain embodiments, the compound for treatment of epileptic conditions comprises the general formula (6a″) with R¹⁰ being a C₁-C₃ alkyl or hydrogen, R¹¹ being a substituted or unsubstituted aryl or a substituted or unsubstituted heteroaryl, in some instances R¹¹ being in case of formula (6a″) an substituted or unsubstituted aryl, in some instances phenyl R¹¹ being in case of formula (7a″) a substituted or unsubstituted heteroaryl, in some instances pyridine, R⁵ being Cl, Br or F.

In certain embodiments, the compound comprises the general formula (7a″) with R¹¹ being a substituted or unsubstituted aryl or a substituted or unsubstituted heteroaryl, in some instances R¹¹ being in case of formula (6a″) an substituted or unsubstituted aryl, in some instances phenyl R¹¹ being in case of formula (7a″) a substituted or unsubstituted heteroaryl, in some instances pyridine, R⁵ being C₂ alkynyl.

In certain embodiments, the compound comprises the general formula (2a″), (3a″), (4a″), (5a″) or (5b″) with in case of formula (2a″) R¹ being or an unsubstituted phenyl, a substituted phenyl comprising at least one —F as a substituent, R⁷ being an unsubstituted C₁-C₆ alkyl, in some instances tert-butyl, or an unsubstituted C₃-C₈ cycloalkyl, in some instances a C₄-cycloalkyl, and R⁸ being —O—CH₂—R⁴, with R⁴ being a substituted or unsubstituted C₄ heteroaryl, in some instances a substituted or unsubstituted triazole, in case of formula (3a″) R¹ being an unsubstituted biphenyl, a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, R⁸ being an unsubstituted C₁-C₆ alcohol, in some instances a C₄ alcohol, in case of formula (4a″) R¹ being —(C═O)—R³, with R³ being an unsubstituted C₆ heteroaryl, in some instances R³ being pyridine, and R⁹ being an unsubstituted C₆ heteroaryl, in some instances pyridine, in case of formula (5a″) R¹ being an unsubstituted biphenyl, a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, R⁷ being an unsubstituted C₁-C₆ alcohol, in some instances a C₄ alcohol, in case of formula (5a″) R¹ being an unsubstituted biphenyl, a substituted biphenyl comprising at least one —CN as a substituent, in some instances on the phenyl moiety not connected to the parent moiety, wherein in some instances one phenyl moiety comprises additionally at least one —F as a substituent, in some instances each phenyl moiety comprises additionally at least one —F as a substituent, R⁷ being an unsubstituted C₁-C₆ alcohol, in some instances a C₄ alcohol, R⁹ being a halogen, in some instances —F.

A GABA_{A} receptor modulator that can be used in the treatment of epileptic conditions can be a compound of general formula (8)

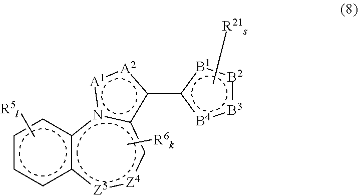

where
Z⁴ and Z⁵ are independently of each other —C, —N, —S or —O,
A¹ and A² are independently of each other —C, —N or —(C═O)—O—R⁷, with R⁷ being an alkyl,
B¹, B², B³, and B⁴ are independently of each other —C, —N, or —O,
l of R⁵ₗ is 1 or 2,
each R⁵ is independently from each other a C₁-C₄ alkynyl or a halogen, in some instances —Cl,
k of R⁶ₖ is 1, 2, 3 or 4, in some instances 1 or 2,
each R⁶ is independently from each other a substituted or unsubstituted aryl, a substituted or unsubstituted heteroaryl, a substituted or unsubstituted C₁-C₃ alkyl, oxygen or hydrogen,
s of R²¹ₛ is 1, 2, 3 or 4, or
each R²¹ is independently hydrogen or C₁-C₆ alkyl.

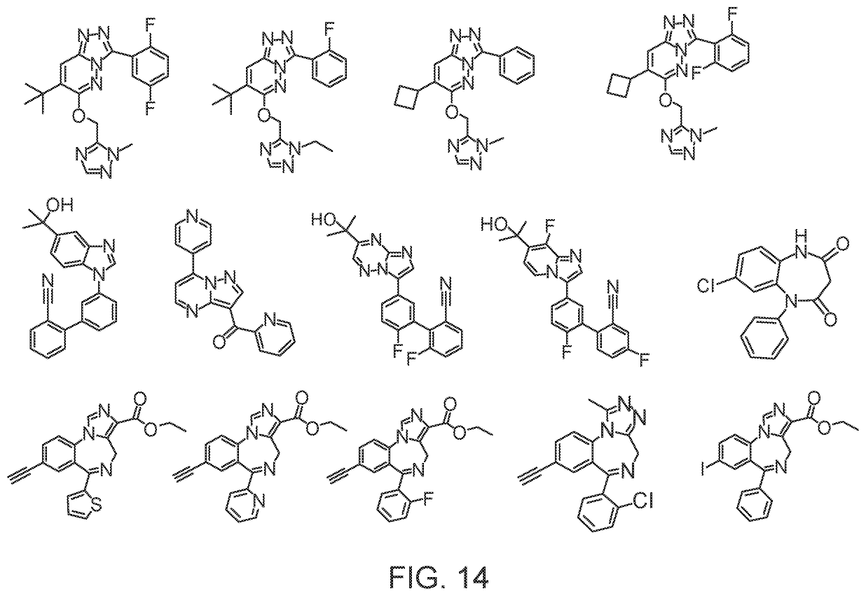

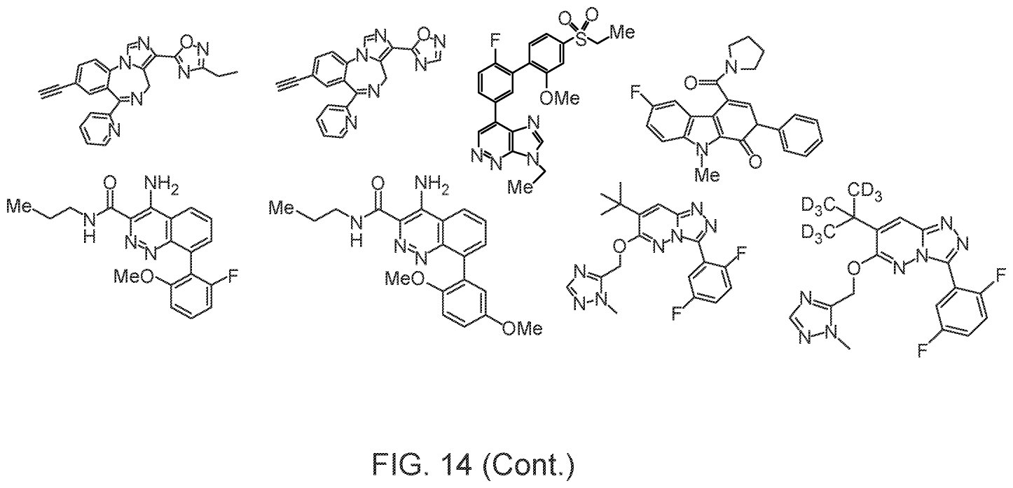

In certain embodiments, the compound is selected from the compounds depicted in FIG. 14, such as L-838417, TPA023 (MK-0777), TPA123, MRK-409(MK-0343), NS11394, Ocinaplon(DOV-273547), TPA023B, TP003, N-Desmethylclobazam 1, 2, 3, 4 and 5, Hz-166, MP-III-080, KRM-II-81, PF-06372865, SL65.1498, AZD7325, AZD6280, L-838417, and CTP-354.

In some cases, a compound is selected from the group consisting of:

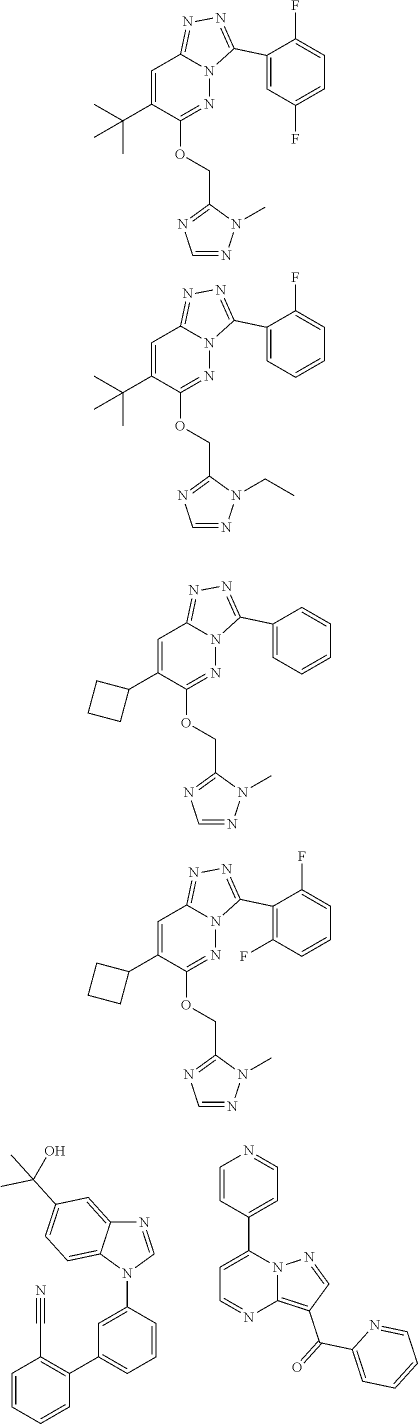

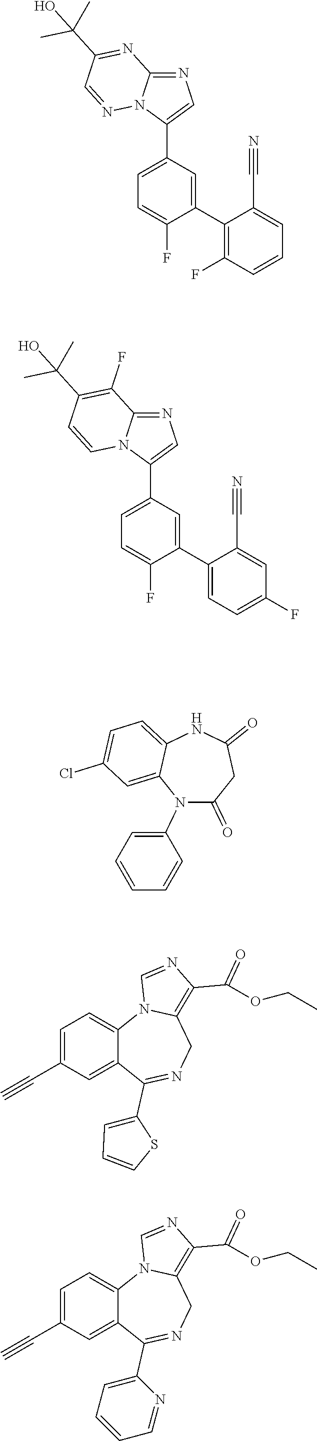

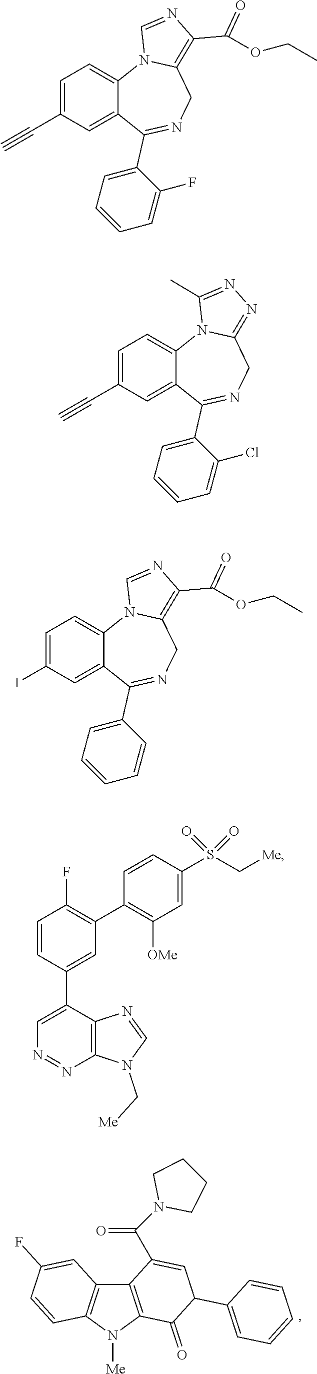

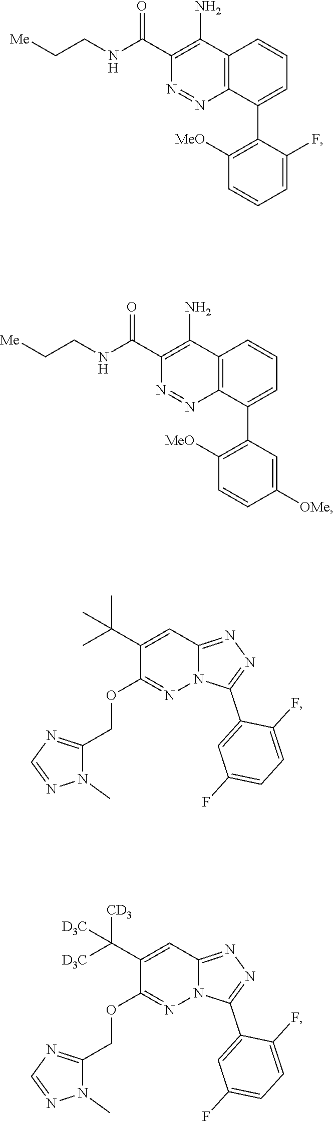 [SMILES: CCCNC(=O)c1nnc2c(-c3c(F)cccc3OC)cccc2c1N ; CCCNC(=O)c1nnc2c(-c3cc(OC)ccc3OC)cccc2c1N ; Cn1ncnc1COc1nn2c(-c3cc(F)ccc3F)nnc2cc1C(C)(C)C ; [2H]C([2H])([2H])C(c1cc2nnc(-c3cc(F)ccc3F)n2nc1OCc1ncnn1C)(C([2H])([2H])[2H])C([2H])([2H])[2H]]

or a salt, prodrug, polymorph, solvate, ester, stereoisomer, or derivative thereof.

In some cases, a compound can be

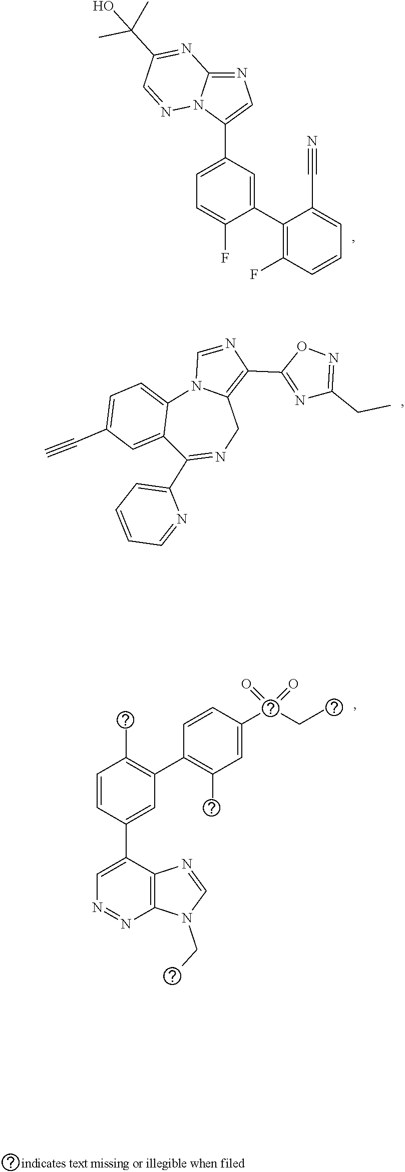

or a salt, prodrug, polymorph, solvate, ester, stereoisomer, or a derivative thereof.

As demonstrated in the Examples below, exemplary GABA_{A} receptor modulators Compound 1 and Compound 2 are effective at preventing heat induced seizures. The structure of Compound 1 and Compound 2 are recited below:

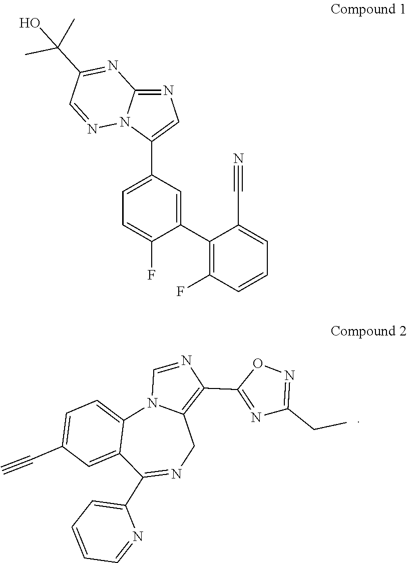 [SMILES: CC(C)(O)c1cnn2c(-c3ccc(F)c(-c4c(F)cccc4C#N)c3)cnc2n1 ; C#Cc1ccc2c(c1)C(c1ccccn1)=NCc1c(-c3nc(CC)no3)ncn1-2]

As described herein, GABA_{A} receptor modulators such as Compound 1 and Compound 2 are expected to be effective at treating epileptic conditions due to their modulation of the GABA_{A} receptor.

Also disclosed herein are pharmaceutically acceptable salt forms of GABA_{A} receptor modulators. A salt can comprise a counterion selected from the group consisting of acetate, benzoate, bitartrate, aspartate, formate, bromide, chloride, iodide, fumarate, citrate, maleate, nitrate, salicylate, succinate, sulfate, phosphate, besylate, hippurate, naphthoate, napsylate, sulfosalicylate, tosylate, octanoate, oleate, pamoate, stearate, propionate, hexanoate, decanoate, aspartate, bitartrate, salicylate, napsylate, or sulfite, and any combination thereof.

Also disclosed herein are polymorphs of a GABA_{A} receptor modulator. A polymorph can include a free base polymorph of a GABA_{A} receptor modulator described herein, or a salt polymorph of a GABA_{A} receptor modulator, or a co-crystal of a GABA_{A} receptor modulator described herein. In some cases, a polymorph can have improved solubility, improved oral bioavailability, more consistent oral bioavailability, improved stability, improved manufacturability, and corresponding improved formulations. Such a polymorph can be prepared by crystalizing or co-crystalizing a GABA_{A} receptor modulator free base or salt form in a crystallization solvent, such as ethyl acetate, methyl ethyl ketone, 2-methyl butanone, dimethyl sulfoxide, dimethylformamide, dimethyl acetamide, acetone, water, tetrahydrofuran (THF), 2-methyl-THF, isopropyl acetate (IPAC), acetonitrile, or dichloromethane.

Preparation of a polymorph can be confirmed by collecting a diffraction pattern of the crystal or co-crystal. In some cases, X-ray Powder Diffraction (XRPD) can be used to collect the diffraction pattern. Exemplary XRPD parameters are provided below.

| Parameters | Settings/Values |
|---|---|
| X-Ray wavelength | Cu: K- Alpha (λ = 1.54179Å) |
| X-Ray tube setting | Voltage: 40 kV; Current: 40 mA |
| Scan scope | 3 to 40 deg |
| Sample rotation speed | 15 rpm |
| Scanning rate | 10 deg./min |

In some exemplary cases, a polymorph of Compound 1 can be administered, such as a polymorph of Compound 1 with an XRPD pattern having characteristic peak locations of at least three of the values selected from the group consisting of: about 6.4, 7.5, 10.2, 12.7, 13.3, 14.5, 16.0, 17.1, 17.4, 17.9, 18.5, 19.1, 19.7, 20.3, 20.9, 21.5, 22.6, 23.7, 26.2, 26.7, 26.9, 27.5, 28.4, 30.2, and 32.1±0.2 degrees, 2-theta, about 5.4, 10.8, 12.3, 12.6, 13.5, 14.8, 15.9, 16.3, 16.4, 17.3, 17.8, 19.3, 20.4, 21.5, 21.7, 22.7, 23.4, 24.4, 24.7, 25.0, 26.1, 26.6, 27.0, 27.2, 27.5, 28.4, 28.7, 29.0, 29.6, 30.2, and 32.3±0.2-degrees, 2-theta, about 7.0, 12.4, 12.6, 13.0, 14.1, 15.4, 15.7, 16.3, 17.5, 18.3, 19.0, 21.0, 22.3, 23.0, and 24.9±0.2 degrees, 2-theta; when measured using the XRPD parameters described above.

### Pharmaceutical Compositions

Also disclosed herein are pharmaceutical compositions comprising a GABA_{A} receptor modulator as described herein. In some embodiments, a pharmaceutical composition can comprise 1, 2, 3, 4, 5, 6, 7, 8, 9, 10, 11, 12, 13, 14, 15, 16, 17, 18, 19, 20 or more GABA_{A} receptor modulators disclosed herein.

In some instances, a pharmaceutical composition can comprise a GABA_{A} receptor modulator described herein and at least one of: an excipient, a diluent, or a carrier. In some cases, a GABA_{A} receptor modulator described herein can be dissolved or suspended in a diluent or carrier.

In some embodiments, a pharmaceutical composition can comprise an excipient. An excipient can be an excipient described in the Handbook of Pharmaceutical Excipients, American Pharmaceutical Association (1986).

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In some embodiments an excipient can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical composition.

In some embodiments an excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments a pharmaceutical composition can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

The binders that can be used in a pharmaceutical composition can be selected from starches such as potato starch, corn starch, wheat starch; sugars such as sucrose, glucose, dextrose, lactose, maltodextrin; natural and synthetic gums; gelatin; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments a pharmaceutical composition can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical composition can be selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulfates (such as sodium lauryl sulfate, magnesium lauryl sulfate), sodium chloride, sodium benzoate, sodium acetate and talc or a combination thereof.

In some embodiments a pharmaceutical composition can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments a pharmaceutical composition can comprise a disintegrant as an excipient. In some embodiments a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments a disintegrant can be an effervescent disintegrant. Non-limiting examples of suitable effervescent disintegrants can include sodium bicarbonate in combination with citric acid, and sodium bicarbonate in combination with tartaric acid.

In some embodiments an excipient can comprise a flavoring agent. Flavoring agents incorporated into an outer layer can be chosen from synthetic flavor oils and flavoring aromatics; natural oils; extracts from plants, leaves, flowers, and fruits; and combinations thereof. In some embodiments a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments an excipient can comprise a sweetener. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

In some instances, a pharmaceutical composition can comprise a coloring agent. Non-limiting examples of suitable color agents can include food, drug and cosmetic colors (FD&C), drug and cosmetic colors (D&C), and external drug and cosmetic colors (Ext. D&C). A coloring agent can be used as dyes or their corresponding lakes.

In some instances, a pharmaceutical composition can comprise a diluent. Non-limiting examples of diluents can include water, glycerol, methanol, ethanol, and other similar biocompatible diluents. In some cases, a diluent can be an aqueous acid such as acetic acid, citric acid, maleic acid, hydrochloric acid, phosphoric acid, nitric acid, sulfuric acid, or similar. In some instances, a diluent can be used to titrate a pH of a compound to a pH such as physiological pH to produce a salt as described above. In other cases, a diluent can be selected from a group comprising alkaline metal carbonates such as calcium carbonate; alkaline metal phosphates such as calcium phosphate; alkaline metal sulfates such as calcium sulfate; cellulose derivatives such as cellulose, microcrystalline cellulose, cellulose acetate; magnesium oxide, dextrin, fructose, dextrose, glyceryl palmitostearate, lactitol, caoline, lactose, maltose, mannitol, simethicone, sorbitol, starch, pregelatinized starch, talc, xylitol and/or anhydrates, hydrates and/or pharmaceutically acceptable derivatives thereof or combinations thereof.

In other embodiments, a pharmaceutical composition can comprise a surfactant. Surfactants can be selected from, but not limited to, polyoxyethylene sorbitan fatty acid esters (polysorbates), sodium lauryl sulfate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids or a combination thereof.

A pharmaceutical composition disclosed herein can be formulated into a variety of forms and administered by a number of different means. A pharmaceutical composition can be administered orally, rectally, or parenterally, in formulations containing conventionally acceptable carriers, adjuvants, and vehicles as desired. The term “parenteral” as used herein can include subcutaneous, intravenous, intramuscular, or intrasternal injection and infusion techniques. Administration can include injection or infusion, including intra-arterial, intracardiac, intracerebroventricular, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration. In some exemplary embodiments, a route of administration can be via an injection such as an intramuscular, intravenous, subcutaneous, or intraperitoneal injection.

Solid dosage forms for oral administration can include capsules, tablets, caplets, pills, troches, lozenges, powders, and granules. A capsule can comprise a core material comprising a nutritive protein or composition and a shell wall that encapsulates a core material. In some embodiments a core material can comprise at least one of a solid, a liquid, and an emulsion. In some embodiments a shell wall material can comprise at least one of a soft gelatin, a hard gelatin, and a polymer. Suitable polymers can include but not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer can function as taste-masking agents.

Tablets, pills, and the like can be compressed, multiply compressed, multiply layered, and/or coated. A coating can be single or multiple. In some embodiments, a coating material can comprise at least one of a saccharide, a polysaccharide, and glycoproteins extracted from at least one of a plant, a fungus, and a microbe. Non-limiting examples can include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments a coating material can comprise a protein. In some embodiments, a coating material can comprise at least one of a fat and/or an oil. In some embodiments the at least one of a fat and/or an oil can be high temperature melting. In some embodiments the at least one of a fat and/or an oil can be hydrogenated or partially hydrogenated. In some embodiments the at least one of a fat and/or an oil can be derived from a plant. In some embodiments the at least one of a fat and/or an oil can comprise at least one of glycerides, free fatty acids, and fatty acid esters. In some embodiments a coating material can comprise at least one edible wax. An edible wax can be derived from animals, insects, or plants. Non-limiting examples can include beeswax, lanolin, bayberry wax, carnauba wax, and rice bran wax. Tablets and pills can additionally be prepared with enteric coatings.

Liquid formulations can include a syrup (for example, an oral formulation), an intravenous formulation, an intranasal formulation, an ocular formulation (e.g. for treating an eye infection), an otic formulation (e.g. for treating an ear infection), an ointment, a cream, an aerosol, and the like. In some instances, a combination of various formulations can be administered. In some embodiments, a tablet, pill, and the like can be formulated for an extended release profile. In some embodiments, a composition can be formulated to increase the shelf stability when stored in a closed container under standard ambient conditions.

Dosing and Administration

In one aspect, described herein is a method of treating an epileptic condition in a subject, comprising administering to the subject a herein described GABA_{A} receptor modulator compound or a pharmaceutical composition comprising the compound. In some cases, a GABA_{A} receptor modulator, salt thereof, or pharmaceutical composition comprising a GABA_{A} receptor modulator or salt thereof described herein can be administered at a dose of from about 0.01 mg to about 1000 mg, from about 0.1 mg to about 1000 mg, from about 0.2 mg to about 1000 mg, from about 0.3 mg to about 1000 mg, from about 0.4 mg to about 1000 mg, from about 0.5 mg to about 1000 mg, from about 0.6 mg to about 1000 mg, from about 0.7 mg to about 1000 mg, from about 0.8 mg to about 1000 mg, from about 0.9 mg to about 1000 mg, from about 1 mg to about 1000 mg, from about 2 mg to about 1000 mg, from about 3 mg to about 1000 mg, from about 4 mg to about 1000 mg, from about 5 mg to about 1000 mg, from about 6 mg to about 1000 mg, from about 7 mg to about 1000 mg, from about 8 mg to about 1000 mg, from about 9 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 35 mg to about 1000 mg, from about 40 mg to about 1000 mg, from about 45 mg to about 1000 mg, from about 50 mg to about 1000 mg, from about 55 mg to about 1000 mg, from about 60 mg to about 1000 mg, from about 65 mg to about 1000 mg, from about 70 mg to about 1000 mg, from about 75 mg to about 1000 mg, from about 80 mg to about 1000 mg, from about 85 mg to about 1000 mg, from about 90 mg to about 1000 mg, from about 95 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg. In some embodiments, a GABA_{A} receptor modulator or salt thereof is administered at a dose of 1 mg to 5 mg. In some embodiments, a GABA_{A} receptor modulator or salt thereof is administered at a dose of 1 mg to 3 mg. In some embodiments, a GABA_{A} receptor modulator or salt thereof is administered at a dose of 1.5 mg to 2.5 mg. In some embodiments, a GABA_{A} receptor modulator or salt thereof is administered at a dose of 1.9 mg to 2.1 mg. In some embodiments, a GABA_{A} receptor modulator or salt thereof is administered at a dose of 1.8 mg to 2.2 mg. In some embodiments, a GABA_{A} receptor modulator or salt thereof is administered at a dose of 0.5 mg to 5 mg. In some embodiments, a GABA_{A} receptor modulator or salt thereof is administered at a dose of 0.5 mg to 4 mg. In some embodiments, a GABA_{A} receptor modulator or salt thereof is administered at a dose of 0.1 mg to 10 mg. In some embodiments, a GABA_{A} receptor modulator or salt thereof is administered at a dose of 0.01 mg to 10 mg. In some embodiments, a GABA_{A} receptor modulator or salt thereof is administered at a dose of 0.01 mg to 5 mg. In some embodiments, a GABA_{A} receptor modulator or salt thereof is administered at a dose of 0.1 mg to 20 mg. In some embodiments, a GABA_{A} receptor modulator or salt thereof is administered at a dose of 0.1 mg to 15 mg. In some embodiments, a GABA_{A} receptor modulator or salt thereof is administered at a dose of 1 mg to 8 mg. In some embodiments, a GABA_{A} receptor modulator or salt thereof is administered at a dose of 0.5 mg to 10 mg. In some embodiments, a GABA_{A} receptor modulator or salt thereof is administered at a dose of 0.25 mg to 5 mg. In some embodiments, a GABA_{A} receptor modulator or salt thereof is administered at a dose of 2 mg to 5 mg. In some embodiments, a GABA_{A} receptor modulator or salt thereof is administered at a dose of 2 mg to 10 mg. In some embodiments, a GABA_{A} receptor modulator or salt thereof is administered at a dose of 3 mg to 5 mg. In some embodiments, a GABA_{A} receptor modulator or salt thereof is administered at a dose of 2 mg to 4 mg. In some embodiments, a GABA_{A} receptor modulator or salt thereof is administered at a dose of 6 mg to 7 mg. In some embodiments, a GABA_{A} receptor modulator or salt thereof is administered at a dose of 5 mg to 15 mg. In some embodiments, a GABA_{A} receptor modulator or salt thereof is administered at a dose of 10 mg to 20 mg.

In some cases, a GABA_{A} receptor modulator, salt thereof, or pharmaceutical composition comprising a GABA_{A} receptor modulator or salt thereof described herein can be administered at a dose of at least about 0.01, 0.02, 0.03, 0.04. 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 mg. In some cases, a GABA_{A} receptor modulator, salt thereof, or pharmaceutical composition comprising a GABA_{A} receptor modulator or salt thereof described herein can be administered at a dose of at most about 0.01, 0.02, 0.03, 0.04. 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 mg. In some cases, a GABA_{A} receptor modulator or salt thereof is administered at a dose of about 0.5 mg. In some cases, a GABA_{A} receptor modulator or salt thereof is administered at a dose of about 1 mg. In some cases, a GABA_{A} receptor modulator or salt thereof is administered at a dose of about 1.5 mg. In some cases, a GABA_{A} receptor modulator or salt thereof is administered at a dose of about 2 mg. In some cases, a GABA_{A} receptor modulator or salt thereof is administered at a dose of about 2.5 mg. In some cases, a GABA_{A} receptor modulator or salt thereof is administered at a dose of about 3 mg. In some cases, a GABA_{A} receptor modulator or salt thereof is administered at a dose of about 3.5 mg. In some cases, a GABA_{A} receptor modulator or salt thereof is administered at a dose of about 4 mg. In some cases, a GABA_{A} receptor modulator or salt thereof is administered at a dose of about 4.5 mg. In some cases, a GABA_{A} receptor modulator or salt thereof is administered at a dose of about 5 mg. In some cases, a GABA_{A} receptor modulator or salt thereof is administered at a dose of about 10 mg. In some cases, a GABA_{A} receptor modulator or salt thereof is administered at a dose of about 15 mg.

In some embodiments, the described GABA_{A} receptor modulator or salt thereof is administered daily. In some embodiments, the described GABA_{A} receptor modulator or salt thereof is administered once daily. In some embodiments, the described GABA_{A} receptor modulator or salt thereof is administered twice daily. In some embodiments, the described GABA_{A} receptor modulator or salt thereof is administered 3 times daily. In some embodiments, the described GABA_{A} receptor modulator or salt thereof is administered 1 to 4 times daily. In some embodiments, the described GABA_{A} receptor modulator or salt thereof is administered once a week.

In some cases, a GABA_{A} receptor modulator, salt thereof, or pharmaceutical composition comprising a GABA_{A} receptor modulator or salt thereof described herein can be administered at a dose with respect to a subject body weight. In some cases, a GABA_{A} receptor modulator, salt thereof, or pharmaceutical composition comprising a GABA_{A} receptor modulator or salt thereof described herein can be administered at a dose of at least 0.0003 mg/kg. In some cases, a GABA_{A} receptor modulator, salt thereof, or pharmaceutical composition comprising a GABA_{A} receptor modulator or salt thereof described herein can be administered at a dose of from about 0.003 mg/kg to about 100 mg/kg, from about 0.003 mg/kg to about 95 mg/kg, from about 0.003 mg/kg to about 90 mg/kg, from about 0.003 mg/kg to about 85 mg/kg, from about 0.003 mg/kg to about 80 mg/kg, from about 0.003 mg/kg to about 75 mg/kg, from about 0.003 mg/kg to about 70 mg/kg, from about 0.003 mg/kg to about 65 mg/kg, from about 0.003 mg/kg to about 60 mg/kg, from about 0.003 mg/kg to about 55 mg/kg, from about 0.003 mg/kg to about 50 mg/kg, from about 0.003 mg/kg to about 45 mg/kg, from about 0.003 mg/kg to about 40 mg/kg, from about 0.003 mg/kg to about 35 mg/kg, from about 0.003 mg/kg to about 30 mg/kg, from about 0.003 mg/kg to about 25 mg/kg, from about 0.003 mg/kg to about 20 mg/kg, from about 0.003 mg/kg to about 15 mg/kg, from about 0.003 mg/kg to about 10 mg/kg, from about 0.003 mg/kg to about 9 mg/kg, from about 0.003 mg/kg to about 8 mg/kg, from about 0.003 mg/kg to about 7 mg/kg, from about 0.003 mg/kg to about 6 mg/kg, from about 0.003 mg/kg to about 5 mg/kg, from about 0.003 mg/kg to about 4 mg/kg, from about 0.003 mg/kg to about 3 mg/kg, from about 0.003 mg/kg to about 2 mg/kg, or from about 0.003 mg/kg to about 1 mg/kg with respect to a body weight of a subject. In some embodiments, a GABA_{A} receptor modulator or salt thereof is administered from 0.003 mg/kg to 10 mg/kg. In some embodiments, a GABA_{A} receptor modulator or salt thereof is administered from 0.0003 mg/kg to 1 mg/kg. In some embodiments, a GABA_{A} receptor modulator or salt thereof is administered from 0.0005 mg/kg to 0.5 mg/kg. In some embodiments, a GABA_{A} receptor modulator or salt thereof is administered from 0.001 mg/kg to 0.5 mg/kg. In some embodiments, a GABA_{A} receptor modulator or salt thereof is administered from 0.001 mg/kg to 0.1 mg/kg. In some embodiments, a GABA_{A} receptor modulator or salt thereof is administered from 0.001 mg/kg to 1 mg/kg. In some embodiments, a GABA_{A} receptor modulator or salt thereof is administered from 0.001 mg/kg to 2 mg/kg. In some embodiments, a GABA_{A} receptor modulator or salt thereof is administered from 0.0003 mg/kg to 0.1 mg/kg. In some embodiments, a GABA_{A} receptor modulator or salt thereof is administered from 0.003 mg/kg to 2 mg/kg. In some embodiments, a GABA_{A} receptor modulator or salt thereof is administered from 0.003 mg/kg to 1 mg/kg. In some embodiments, a GABA_{A} receptor modulator or salt thereof is administered from 0.003 mg/kg to 0.3 mg/kg. In some embodiments, a GABA_{A} receptor modulator or salt thereof is administered from 0.003 mg/kg to 0.1 mg/kg. In some embodiments, a GABA_{A} receptor modulator or salt thereof is administered from 0.003 mg/kg to 0.03 mg/kg. In some embodiments, a GABA_{A} receptor modulator or salt thereof is administered from 0.01 mg/kg to 10 mg/kg. In some embodiments, a GABA_{A} receptor modulator or salt thereof is administered from 0.01 mg/kg to 2 mg/kg. In some embodiments, a GABA_{A} receptor modulator or salt thereof is administered from 0.01 mg/kg to 1 mg/kg. In some embodiments, a GABA_{A} receptor modulator or salt thereof is administered from 0.01 mg/kg to 0.3 mg/kg. 1 mg/kg. In some embodiments, a GABA_{A} receptor modulator or salt thereof is administered from 0.01 mg/kg to 0.05 mg/kg. In some embodiments, a GABA_{A} receptor modulator or salt thereof is administered from 0.01 mg/kg to 0.1 mg/kg. In some embodiments, a GABA_{A} receptor modulator or salt thereof is administered from 0.01 mg/kg to 0.03 mg/kg. In some embodiments, a GABA_{A} receptor modulator or salt thereof is administered from 0.03 mg/kg to 10 mg/kg. In some embodiments, a GABA_{A} receptor modulator or salt thereof is administered from 0.03 mg/kg to 2 mg/kg. In some embodiments, a GABA_{A} receptor modulator or salt thereof is administered from 0.03 mg/kg to 1 mg/kg. In some embodiments, a GABA_{A} receptor modulator or salt thereof is administered from 0.03 mg/kg to 0.3 mg/kg. In some embodiments, a GABA_{A} receptor modulator or salt thereof is administered from 0.03 mg/kg to 0.1 mg/kg. In some embodiments, a GABA_{A} receptor modulator or salt thereof is administered from 0.05 mg/kg to 10 mg/kg. In some embodiments, a GABA_{A} receptor modulator or salt thereof is administered from 0.05 mg/kg to 2 mg/kg. In some embodiments, a GABA_{A} receptor modulator or salt thereof is administered from 0.05 mg/kg to 1 mg/kg. In some embodiments, a GABA_{A} receptor modulator or salt thereof is administered from 0.05 mg/kg to 0.3 mg/kg. In some embodiments, a GABA_{A} receptor modulator or salt thereof is administered from 0.05 mg/kg to 0.1 mg/kg. In some embodiments, a GABA_{A} receptor modulator or salt thereof is administered from 0.1 mg/kg to 10 mg/kg. In some embodiments, a GABA_{A} receptor modulator or salt thereof is administered from 0.1 mg/kg to 2 mg/kg. In some embodiments, a GABA_{A} receptor modulator or salt thereof is administered from 0.1 mg/kg to 1 mg/kg. In some embodiments, a GABA_{A} receptor modulator or salt thereof is administered from 0.1 mg/kg to 0.3 mg/kg. In some embodiments, a GABA_{A} receptor modulator or salt thereof is administered from 0.1 mg/kg to 0.5 mg/kg. In some embodiments, a GABA_{A} receptor modulator or salt thereof is administered from 0.1 mg/kg to 0.05 mg/kg. In some embodiments, a GABA_{A} receptor modulator or salt thereof is administered at about 0.01 mg/kg. In some embodiments, a GABA_{A} receptor modulator or salt thereof is administered at about 0.02 mg/kg. In some embodiments, a GABA_{A} receptor modulator or salt thereof is administered at about 0.03 mg/kg. In some embodiments, a GABA_{A} receptor modulator or salt thereof is administered at about 0.04 mg/kg. In some embodiments, a GABA_{A} receptor modulator or salt thereof is administered at about 0.05 mg/kg. In some embodiments, a GABA_{A} receptor modulator or salt thereof is administered at about 0.06 mg/kg. In some embodiments, a GABA_{A} receptor modulator or salt thereof is administered at about 0.07 mg/kg. In some embodiments, a GABA_{A} receptor modulator or salt thereof is administered at about 0.08 mg/kg. In some embodiments, a GABA_{A} receptor modulator or salt thereof is administered at about 0.09 mg/kg. In some embodiments, a GABA_{A} receptor modulator or salt thereof is administered at about 0.1 mg/kg.

In some cases, a GABA_{A} receptor modulator, salt thereof, or pharmaceutical composition comprising a GABA_{A} receptor modulator or salt thereof described herein can be administered at a dose of less than 0.0005 mg/kg, less than 0.001 mg/kg, less than 0.002 mg/kg, less than 0.003 mg/kg, less than 0.004 mg/kg, less than 0.005 mg/kg, less than 0.006 mg/kg, less than 0.007 mg/kg, less than 0.008 mg/kg, less than 0.009 mg/kg, less than 0.01 mg/kg, less than 0.02 mg/kg, less than 0.03 mg/kg, less than 0.04 mg/kg, less than 0.05 mg/kg, less than 0.06 mg/kg, less than 0.07 mg/kg, less than 0.08 mg/kg, less than 0.09 mg/kg, less than 0.1 mg/kg, less than 0.11 mg/kg, less than 0.12 mg/kg, less than 0.13 mg/kg, less than 0.14 mg/kg, less than 0.15 mg/kg, less than 0.16 mg/kg, less than 0.17 mg/kg, less than 0.18 mg/kg, less than 0.19 mg/kg, less than 0.2 mg/kg, less than 0.21 mg/kg, less than 0.22 mg/kg, less than 0.23 mg/kg, less than 0.24 mg/kg, less than 0.25 mg/kg, less than 0.26 mg/kg, less than 0.27 mg/kg, less than 0.28 mg/kg, less than 0.29 mg/kg, less than 0.3 mg/kg, less than 0.31 mg/kg, less than 0.32 mg/kg, less than 0.33 mg/kg, less than 0.34 mg/kg, less than 0.35 mg/kg, less than 0.36 mg/kg, less than 0.37 mg/kg, less than 0.38 mg/kg, less than 0.39 mg/kg, less than 0.4 mg/kg, less than 0.41 mg/kg, less than 0.42 mg/kg, less than 0.43 mg/kg, less than 0.44 mg/kg, less than 0.45 mg/kg, less than 0.46 mg/kg, less than 0.47 mg/kg, less than 0.48 mg/kg, less than 0.49 mg/kg, less than 0.5 mg/kg, less than 0.51 mg/kg, less than 0.52 mg/kg, less than 0.53 mg/kg, less than 0.54 mg/kg, less than 0.55 mg/kg, less than 0.56 mg/kg, less than 0.57 mg/kg, less than 0.58 mg/kg, less than 0.59 mg/kg, less than 0.6 mg/kg, less than 0.61 mg/kg, less than 0.62 mg/kg, less than 0.63 mg/kg, less than 0.64 mg/kg, less than 0.65 mg/kg, less than 0.66 mg/kg, less than 0.67 mg/kg, less than 0.68 mg/kg, less than 0.69 mg/kg, less than 0.7 mg/kg, less than 0.71 mg/kg, less than 0.72 mg/kg, less than 0.73 mg/kg, less than 0.74 mg/kg, less than 0.75 mg/kg, less than 0.76 mg/kg, less than 0.77 mg/kg, less than 0.78 mg/kg, less than 0.79 mg/kg, less than 0.8 mg/kg, less than 0.81 mg/kg, less than 0.82 mg/kg, less than 0.83 mg/kg, less than 0.84 mg/kg, less than 0.85 mg/kg, less than 0.86 mg/kg, less than 0.87 mg/kg, less than 0.88 mg/kg, less than 0.89 mg/kg, less than 0.9 mg/kg, less than 0.91 mg/kg, less than 0.92 mg/kg, less than 0.93 mg/kg, less than 0.94 mg/kg, less than 0.95 mg/kg, less than 0.96 mg/kg, less than 0.97 mg/kg, less than 0.98 mg/kg, less than 0.99 mg/kg, less than 1 mg/kg, less than 2 mg/kg, less than 2.1 mg/kg, less than 2.2 mg/kg, less than 2.3 mg/kg, less than 2.4 mg/kg, less than 2.5 mg/kg, less than 2.6 mg/kg, less than 2.7 mg/kg, less than 2.8 mg/kg, less than 2.9 mg/kg, less than 3 mg/kg, less than 3.1 mg/kg, less than 3.2 mg/kg, less than 3.3 mg/kg, less than 3.4 mg/kg, less than 3.5 mg/kg, less than 3.6 mg/kg, less than 3.7 mg/kg, less than 3.8 mg/kg, less than 3.9 mg/kg, less than 4 mg/kg, less than 4.1 mg/kg, less than 4.2 mg/kg, less than 4.3 mg/kg, less than 4.4 mg/kg, less than 4.5 mg/kg, less than 4.6 mg/kg, less than 4.7 mg/kg, less than 4.8 mg/kg, less than 4.9 mg/kg, less than 5 mg/kg, less than 5.1 mg/kg, less than 5.2 mg/kg, less than 5.3 mg/kg, less than 5.4 mg/kg, less than 5.5 mg/kg, less than 5.6 mg/kg, less than 5.7 mg/kg, less than 5.8 mg/kg, less than 5.9 mg/kg, less than 6 mg/kg, less than 6.1 mg/kg, less than 6.2 mg/kg, less than 6.3 mg/kg, less than 6.4 mg/kg, less than 6.5 mg/kg, less than 6.6 mg/kg, less than 6.7 mg/kg, less than 6.8 mg/kg, less than 6.9 mg/kg, less than 7 mg/kg, less than 7.1 mg/kg, less than 7.2 mg/kg, less than 7.3 mg/kg, less than 7.4 mg/kg, less than 7.5 mg/kg, less than 7.6 mg/kg, less than 7.7 mg/kg, less than 7.8 mg/kg, less than 7.9 mg/kg, less than 8 mg/kg, less than 8.1 mg/kg, less than 8.2 mg/kg, less than 8.3 mg/kg, less than 8.4 mg/kg, less than 8.5 mg/kg, less than 8.6 mg/kg, less than 8.7 mg/kg, less than 8.8 mg/kg, less than 8.9 mg/kg, less than 9 mg/kg, less than 9.1 mg/kg, less than 9.2 mg/kg, less than 9.3 mg/kg, less than 9.4 mg/kg, less than 9.5 mg/kg, less than 9.6 mg/kg, less than 9.7 mg/kg, less than 9.8 mg/kg, less than 9.9 mg/kg, less than 10 mg/kg, less than 11 mg/kg, less than 12 mg/kg, less than 13 mg/kg, less than 14 mg/kg, less than 15 mg/kg, less than 16 mg/kg, less than 17 mg/kg, less than 18 mg/kg, less than 19 mg/kg, less than 20 mg/kg, less than 21 mg/kg, less than 22 mg/kg, less than 23 mg/kg, less than 24 mg/kg, less than 25 mg/kg, less than 26 mg/kg, less than 27 mg/kg, less than 28 mg/kg, less than 29 mg/kg, less than 30 mg/kg, less than 31 mg/kg, less than 32 mg/kg, less than 33 mg/kg, less than 34 mg/kg, less than 35 mg/kg, less than 36 mg/kg, less than 37 mg/kg, less than 38 mg/kg, less than 39 mg/kg, less than 40 mg/kg, less than 41 mg/kg, less than 42 mg/kg, less than 43 mg/kg, less than 44 mg/kg, less than 45 mg/kg, less than 46 mg/kg, less than 47 mg/kg, less than 48 mg/kg, less than 49 mg/kg, or less than 50 mg/kg. In some embodiments, a GABA_{A} receptor modulator or salt thereof is administered at a dose of less than 1 mg/kg. In some embodiments, a GABA_{A} receptor modulator or salt thereof is administered at a dose of less than 0.3 mg/kg. In some embodiments, a GABA_{A} receptor modulator or salt thereof is administered at a dose of less than 0.1 mg/kg. In some embodiments, a GABA_{A} receptor modulator or salt thereof is administered at a dose of less than 0.03 mg/kg. In some embodiments, a GABA_{A} receptor modulator or salt thereof is administered at a dose of less than 0.01 mg/kg.

In some embodiments, a GABA_{A} receptor modulator, salt thereof, or pharmaceutical composition comprising a GABA_{A} receptor modulator or salt thereof described herein can be administered as a liquid formulation. In some embodiments, the described GABA_{A} receptor modulator or salt thereof is administered from 0.01 mg/ml to 100 mg/ml. In some embodiments, the described GABA_{A} receptor modulator or salt thereof is administered from 0.001 mg/ml to 10 mg/ml. In some embodiments, the GABA_{A} receptor modulator or salt thereof is administered from 0.1 mg/ml to 10 mg/ml. In some embodiments, the GABA_{A} receptor modulator or salt thereof is administered from 0.001 mg/ml to 10 mg/ml. In some embodiments, the GABA_{A} receptor modulator or salt thereof is administered from 0.001 mg/ml to 1 mg/ml. In some embodiments, the GABA_{A} receptor modulator or salt thereof is administered from 0.001 mg/ml to 0.5 mg/ml. In some embodiments, the GABA_{A} receptor modulator or salt thereof is administered from 0.5 mg/ml to 5 mg/ml. In some embodiments, the GABA_{A} receptor modulator or salt thereof is administered from 1 mg/ml to 2 mg/ml. In some embodiments, the GABA_{A} receptor modulator or salt thereof is administered from 0.5 mg/ml to 2 mg/ml. In some embodiments, the GABA_{A} receptor modulator or salt thereof is administered from 0.2 mg/ml to 1 mg/ml. In some embodiments, the GABA_{A} receptor modulator or salt thereof is administered from 0.2 mg/ml to 0.5 mg/ml. In some embodiments, the GABA_{A} receptor modulator or salt thereof is administered from 0.2 mg/ml to 0.4 mg/ml. In some embodiments, the GABA_{A} receptor modulator or salt thereof is administered from 0.1 mg/ml to 0.5 mg/ml. In some embodiments, the GABA_{A} receptor modulator or salt thereof is administered from 0.1 mg/ml to 0.3 mg/ml. In some embodiments, the GABA_{A} receptor modulator or salt thereof is administered from 0.05 mg/ml to 0.5 mg/ml. In some embodiments, the GABA_{A} receptor modulator or salt thereof is administered at about 0.1 mg/ml. In some embodiments, the GABA_{A} receptor modulator or salt thereof is administered at about 0.25 mg/ml. In some embodiments, the GABA_{A} receptor modulator or salt thereof is administered at about 0.3 mg/ml. In some embodiments, the GABA_{A} receptor modulator or salt thereof is administered at about 0.5 mg/ml. In some embodiments, the GABA_{A} receptor modulator or salt thereof is administered at about 0.75 mg/ml. In some embodiments, the GABA_{A} receptor modulator or salt thereof is administered at about 1 mg/ml. In some embodiments, the GABA_{A} receptor modulator or salt thereof is administered at about 1.25 mg/ml. In some embodiments, the GABA_{A} receptor modulator or salt thereof is administered at about 1.5 mg/ml. In some embodiments, the GABA_{A} receptor modulator or salt thereof is administered at about 2 mg/ml. In some embodiments, the GABA_{A} receptor modulator or salt thereof is administered at about 2.5 mg/ml. In some embodiments, the GABA_{A} receptor modulator or salt thereof is administered at about 5 mg/ml. In some embodiments, the GABA_{A} receptor modulator or salt thereof is administered at about 10 mg/ml. In some embodiments, 0.01 ml to 500 ml of the liquid formulation is administered per day. In some embodiments, 0.05 ml to 250 ml of the liquid formulation is administered per day. In some embodiments, 0.05 ml to 50 ml of the liquid formulation is administered per day. In some embodiments, 0.1 ml to 25 ml of the liquid formulation is administered per day. In some embodiments, 1 ml to 250 ml of the liquid formulation is administered per day. In some embodiments, 1 ml to 200 ml of the liquid formulation is administered per day. In some embodiments, 50 ml to 200 ml of the liquid formulation is administered per day. In some embodiments, 25 ml to 100 ml of the liquid formulation is administered per day. In some embodiments, 5 ml to 50 ml of the liquid formulation is administered per day. In some embodiments, 1 ml to 20 ml of the liquid formulation is administered per day. In some embodiments, 1 ml to 5 ml of the liquid formulation is administered per day.

In some embodiments, described herein is a method of treating an epileptic condition in a subject, comprising administering to the subject a pharmaceutical composition that comprises a compound having a structure of

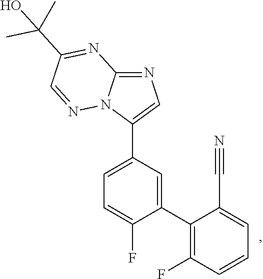 [SMILES: CC(C)(O)c1cnn2c(-c3ccc(F)c(-c4c(F)cccc4C#N)c3)cnc2n1]

or a salt or polymorph thereof, wherein the administering is in an amount effective to treat the epileptic condition in the subject. In some embodiments, the amount effective to treat the epileptic condition comprises a dose of the compound or a salt or polymorph thereof of from about 0.0003 mg to about 1 mg per kg of a body weight of the subject per day. In some embodiments, the amount comprises a dose of the compound or a salt or polymorph thereof of less than 0.3 mg per kg of the body weight of the subject per day. In some embodiments, the amount comprises a dose of the compound or a salt or polymorph thereof of less than 0.1 mg per kg of the body weight of the subject per day. In some embodiments, the amount comprises a dose of the compound or a salt or polymorph thereof of less than 0.03 mg per kg of the body weight of the subject per day.

A person of ordinary skill in the art could extrapolate a dose effective in one animal model for an equivalent dose in another model. For example, a dose shown to be effective in a mouse model can be divided by an allometric scaling factor (i.e. 12) to generate a human equivalent dose.

Administration of a GABA_{A} receptor modulator, salt, or composition can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration of a GABA_{A} receptor modulator, salt, or composition can be performed at least 1, 2, 3, 4, 5, 6, 7, 8, 9, 10, 11, 12, 13, 14, 15, 16, 17, 18, 19, 20, or 21 times a week. In some cases, administration of a GABA_{A} receptor modulator, salt, or composition can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month. In some embodiments, a GABA_{A} receptor modulator or salt thereof is administered to a subject for a period of time. The period of time can be from 1 day to over 30 years. In some embodiments, the period of time is from 1 day to 20 years, from 1 week to 10 years, from 1 month to 5 years, from 1 month to 1 year, from 1 week to 1 year, or any numbers or ranges therebetween.

The invention is further illustrated by the following examples, from which further embodiments and advantages can be drawn. These examples are meant to illustrate the invention but not to limit its scope.

### Example 1—Formulation

Exemplary GABA_{A} receptor modulators were formulated prior to administration. Each modulator was combined with Tylose MH 300 (0.5% w/v), Tween 80 (1% final volume), and 0.9% bacteriostatic saline (to final volume). The resulting suspension was then sonicated for at least 10 minutes at 37° C.

Control solutions of 0.5% Tylose MH 300 (w/v) and 1% Tween 80 (v/v) in 0.9% bacteriostatic saline were also prepared.

### Example 2—Study Design

Animals

Reason for selection of species: The Scn1a^{+/-} knockout mouse ([129xB]F1. Scn1a^{+/-}) is a model subject of DS with a high translational value.

Number: 112 (56 males and 56 females) of first generation of Scn1a^{+/-} heterozygous knockout mice ([129xB]F1. Scn1a^{+/-})

Spare animals: 112 wild type littermates. A portion of spare animals were used to determine PK profiles for the exemplary GABA_{A} receptor modulators.

Strain/Sanitary status: Scn1a^{+/-} heterozygous knockout mice ([129xB]F1. Scn1a^{+/-})

Breeder: Scn1a^{+/-} breeding male mice (129S-Scn1a^{+/-}, n=12) were obtained and set up with C57BL/6J female (n=24) as breeding trios.

Age/Weight: Scn1a^{+/-} heterozygous knockout mice between P14 and P16 were randomly assigned to treatment groups.

Receipt: Upon arrival, breeding female C57BL/6J mice were given a physical examination to ensure that they are healthy. Scn1a^{+/-} breeding male mice (129S-Scn1a^{+/-}, n=12) were quarantined for 2 weeks until cleared by PCR testing.

Acclimation: No acclimation needed. Upon Scn1a^{+/-} heterozygous knockout mice weaned were enrolled in the study and receiving the treatment.

Allocation to groups: Upon weaning, the required number of animals were randomly allocated to the treatment groups (by sex).

Identification: Scn1a^{+/-} heterozygous knockout mice yielded from breeding trios were numbered via toe clipping at age P7-10 in each cage. Cage cards indicated animal numbers within each cage. Wild-type litter mates not designated for PK testing were euthanized. Only Scn1a^{+/-} heterozygous knockout mice ([129xB]F1. Scn1a^{+/-}) were enrolled in the hyperthermia induced seizure study.

Environmental Conditions

From arrival, the animals were housed in a SPF barriered rodent unit. The animal room conditions were set as follows:
temperature: 20-26° C. (68-79° F.)
relative humidity: 50±20%,
light/dark cycle: 12 h/12 h; lights on begins at 0700,
ventilation: approximately 10 to 15 cycles/hour of filtered, non-recycled air.

The corresponding instrumentation and equipment were checked and calibrated at regular intervals. The temperature and relative humidity were recorded continuously (recording devices equipped with alarm systems).

The animal room was disinfected before the arrival of the animals and cleaned regularly thereafter.

Housing

Scn1a^{+/-} breeding male mice (129S-Scn1a^{+/-}) and C57BL/6J females were each housed 4 per cage upon arrival in autoclaved polycarbonate cages equipped with stainless steel lids with Micro-Filter Top and Perforated Retainer containing ¼-in corncob bedding.

One breeding male and 2 females were set up as a breeding trio. Offspring (Scn1a^{+/-} heterozygous knockout mice ([129xB]F1.Scn1a^{+/-} and wildtype) were kept together with their dam (C57BL/6J female) until their enrollment in the study. Wildtype littermates were euthanized upon enrollment of [129xB]F1. Scn1a^{+/-} littermates into the study, unless designated for use in a PK study.

### Example 3—Treatment of Hypothermia-Induced Seizure

Treatment Groups

Rationale for dose level selection—The dose levels were selected based on the results of previous studies.

Between P14 and 16, Scn1a^{+/-} heterozygous knockout mice are randomly assigned to treatment groups (n=7 males, 7 females/treatment group). The Scn1a^{+/-} knockout mouse model was chosen because it is an art accepted animal model for epileptic conditions, including for Dravet Syndrome. Treatments are GABA_{A} receptor modulator 1 (“Compound 1” as a phosphate salt at 0.1, 0.3, 1.0 mg/kg), GABA_{A} receptor modulator 2 (“Compound 2” at 3, 10, 30, 100 mg/kg), vehicle, or Clobazam (“CBZ”) (10 mg/kg; positive control).

Duration

The dose formulations were administered singularly for the study.

Administration

The GABA_{A} receptor modulators were administered intraperitoneally (i.p.) using a 0.5 or 1-ml plastic syringe fitted with a 27 or 30 gauge 0.5″-long beveled needle.

Animals received 10 mL of formulation per kg of body weight as measured on the day of experiment.

Formulations were adjusted to and maintained at delivery conditions (37° C. temperature) throughout the procedure.

Formulations were mixed just prior to administration by inverting their container 2-3 times.

In order to verify the dose concentration, mice were dosed with a test article or control and placed into individual housing cages. A temperature probe was gently inserted into the mouse's rectum and secured by taping the probe's wire to the mouse's tail with the tag end on the mouse's dorsal side. For each group, the insertion of the probe was as follows:
a. For mice administered CBZ, five (5) mins passed before insertion of the probe in order to allow 20-30 min pretreatment time when the state of hyperthermia reached.
b. For mice administered Compound 1, thirty-five (35) mins passed before insertion of the probe in order to allow 50-60 min pretreatment time when the state of hyperthermia is reached.
c. For mice administered Compound, five (5) mins passed before insertion of the probe in order to allow 20-30 min pretreatment time when the state of hyperthermia reached.

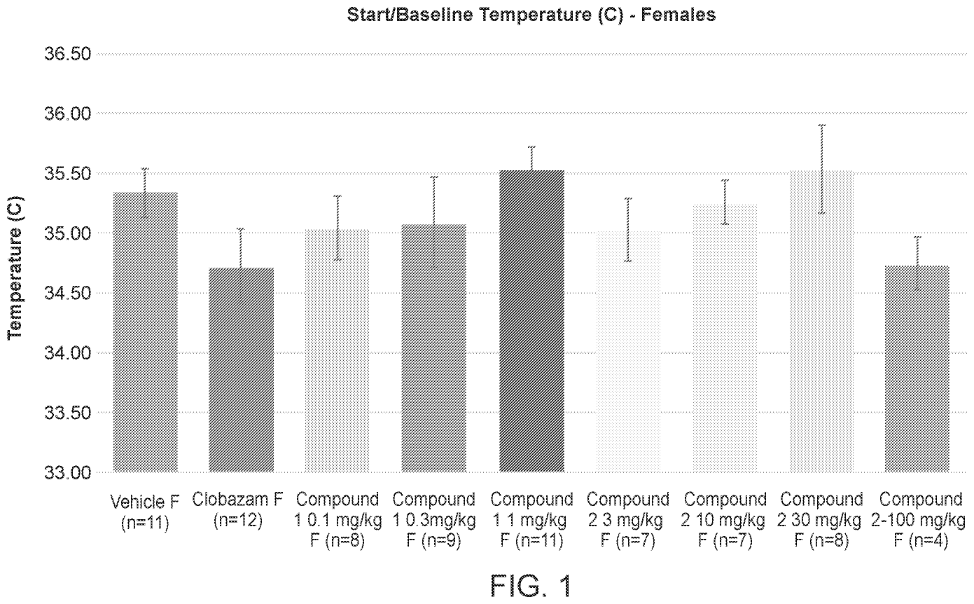

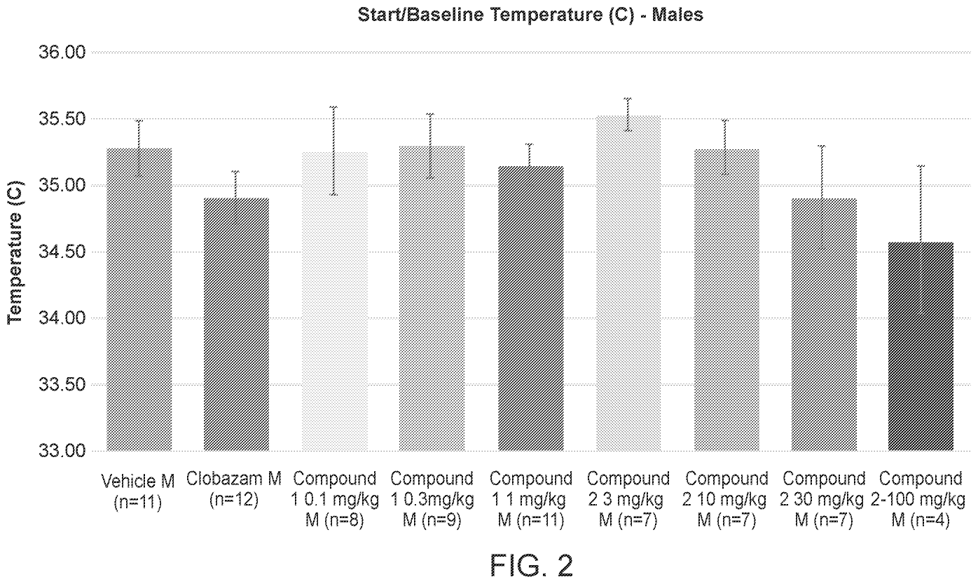

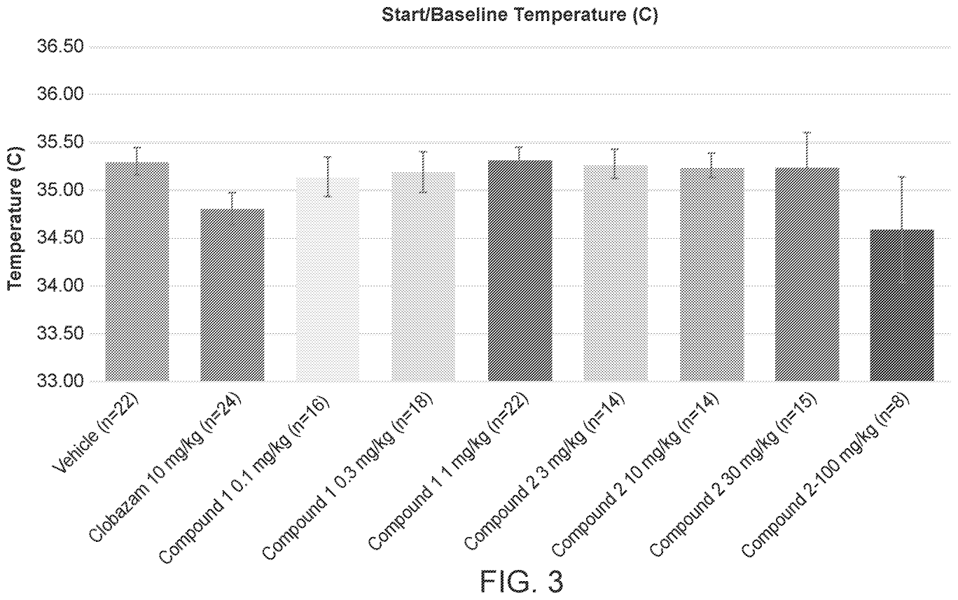

Each mouse was allowed to acclimate to the temperature probe for five (5) minutes before recording the baseline temperature. FIGS. 1-3 depict the starting temperatures of the female mice, the male mice, and the average starting temperatures of all mice prior to inducing the heat-induced seizures.

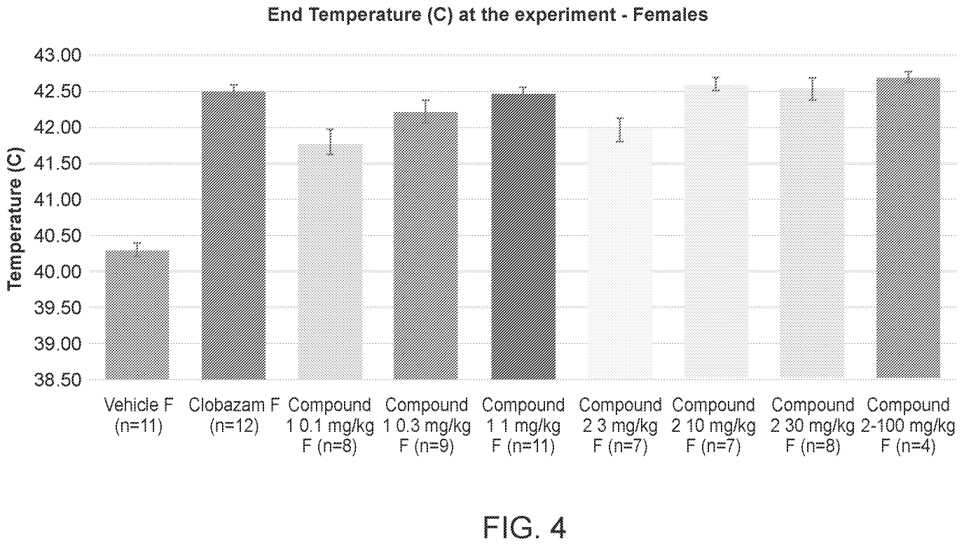

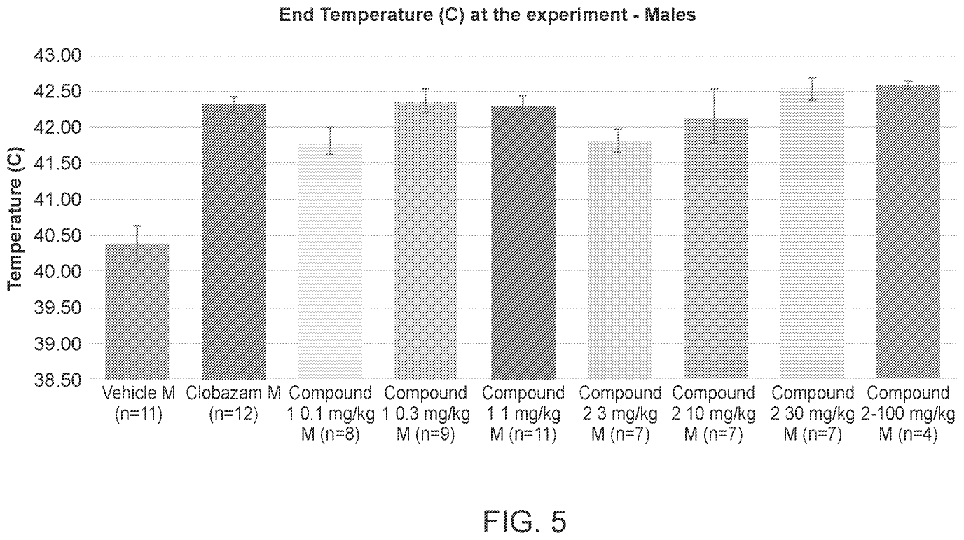

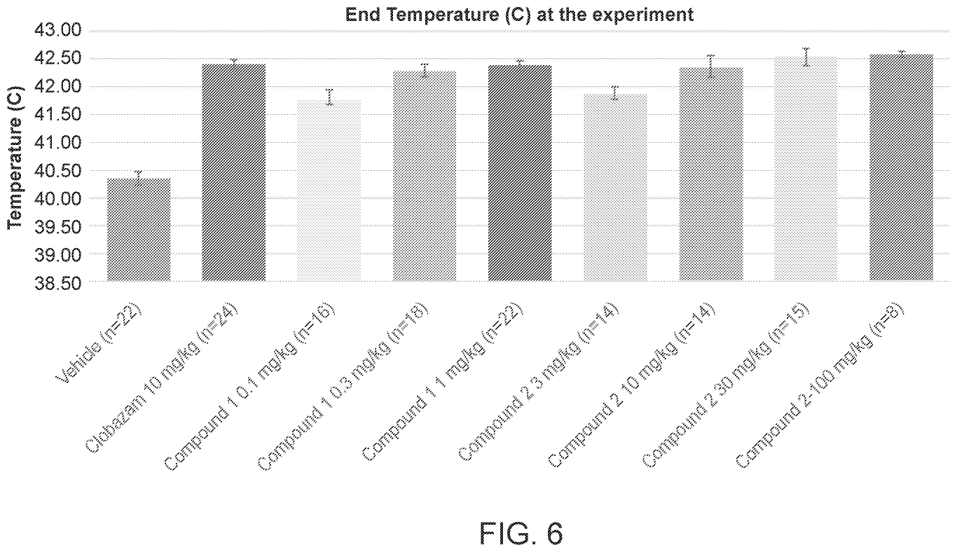

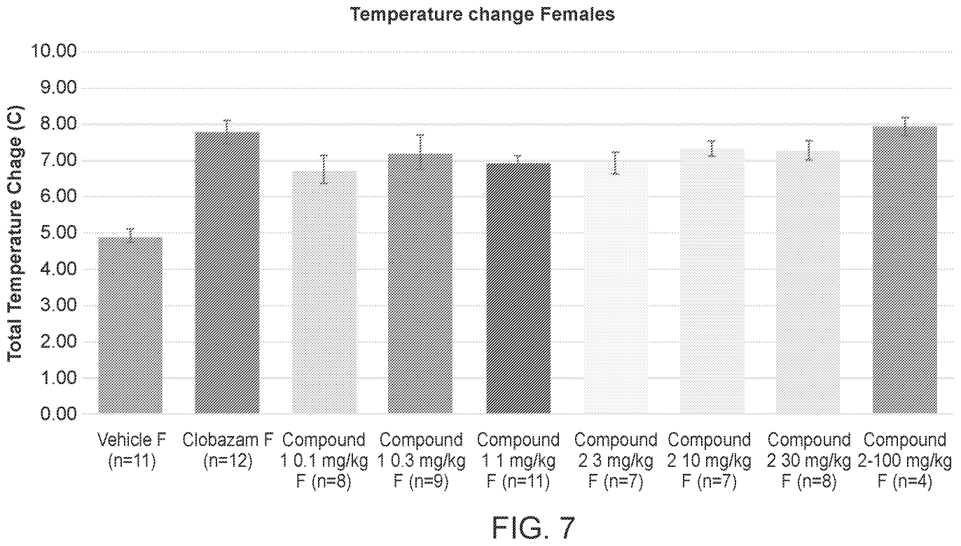

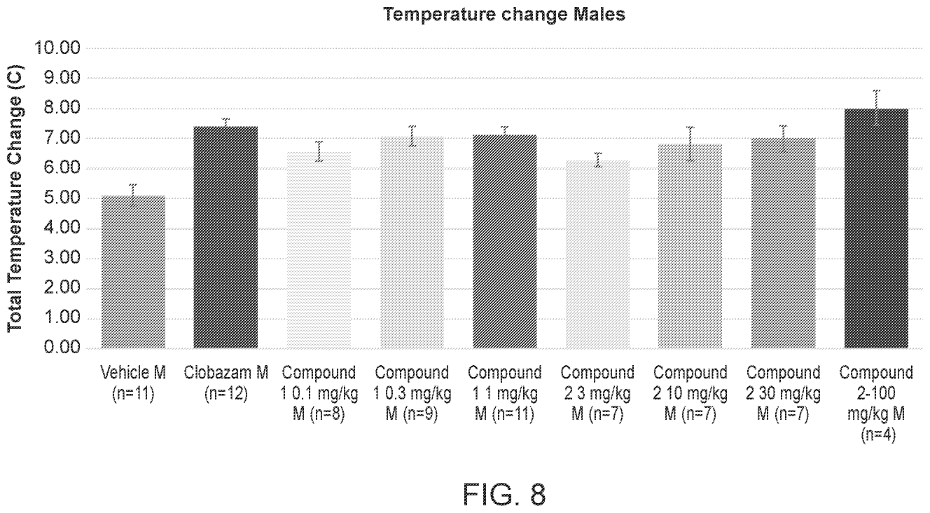

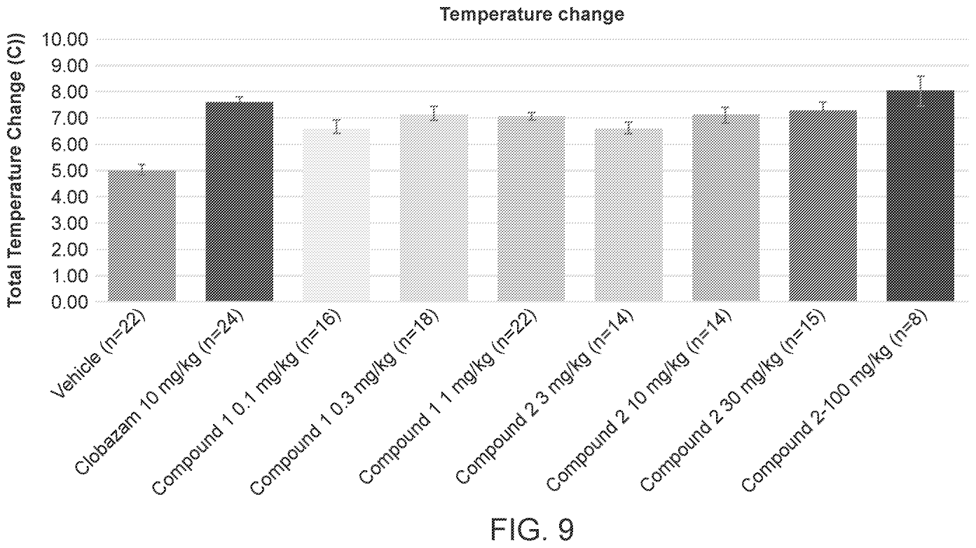

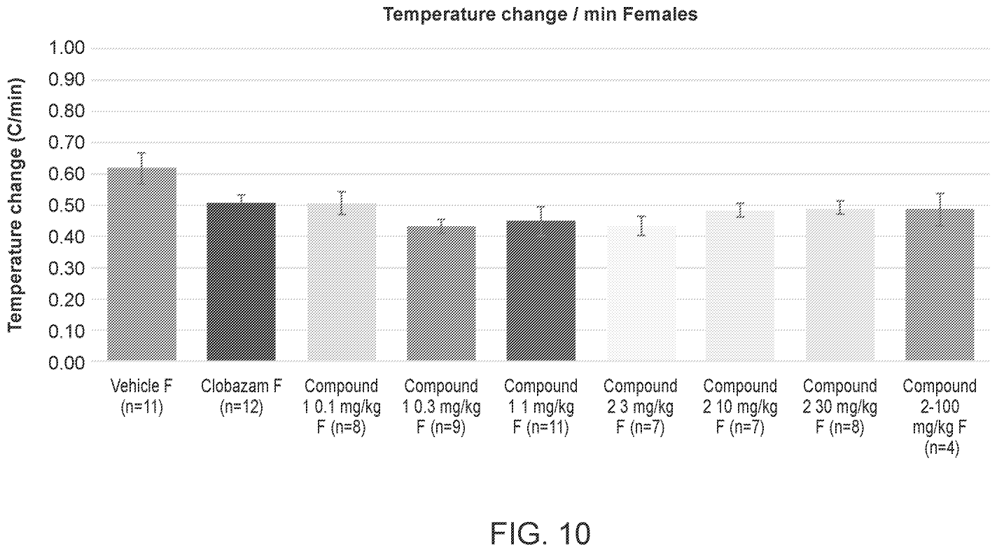

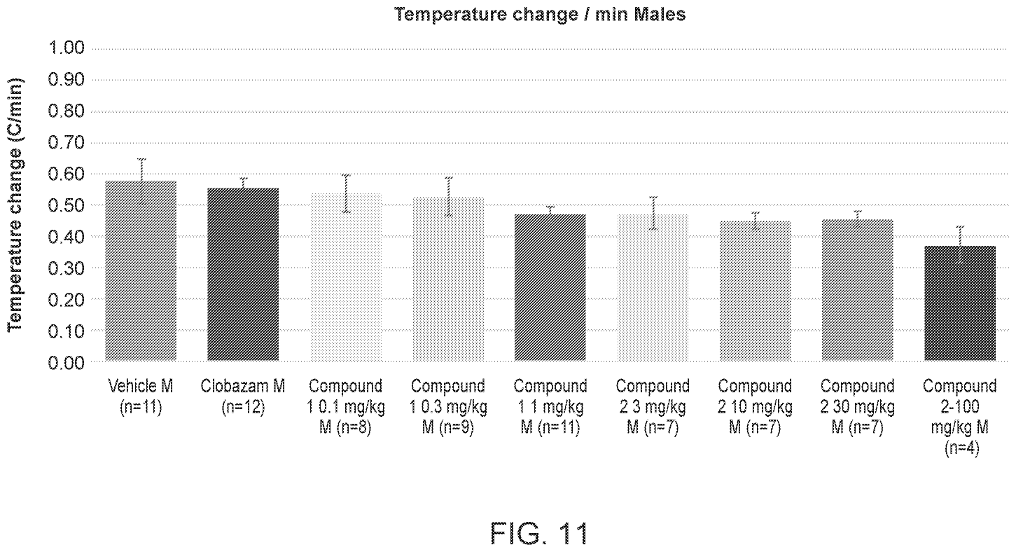

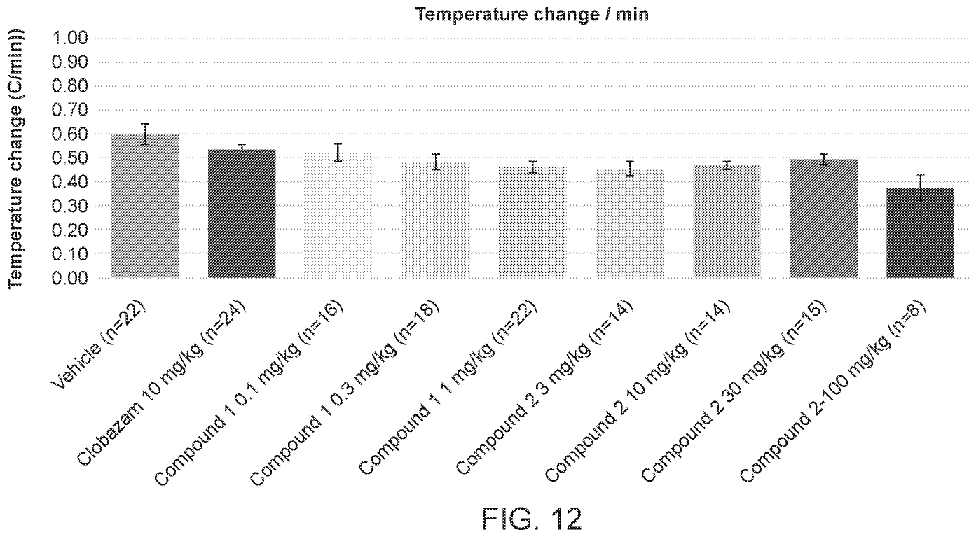

The mouse's core body temperature was raised by about 0.5° C. every 2 min by keeping the mouse on a heating pad until the onset of the first tonic clonic with loss of posture is observed or until a body temperature of 42.5° C. is reached. FIGS. 4-6 depict the final core body temperature of the female mice, the male mice, and the average final core body temperature of all mice after heating. FIGS. 7-9 illustrate the total temperature change for the female mice, the male mice, and average of all mice; while FIGS. 10-12 depict the average temperature change per minute for the female mice, the male mice, and average of all mice. The mouse's body temperature was maintained at 42.5° C. for 3 mins. If no seizure occurred, the mouse was considered seizure-free.

Tables 1-9 below depict the results of administration of control, CBZ, Compound 1, and Compound 2, and their ability to protect against seizure as a function of dosage.

TABLE 1
Control Vehicle (30 minute pretreatment)

| | | | | | | | | | | GTCS |
|---|---|---|---|---|---|---|---|---|---|---|
| | | | | | Base. | End | | Temp | | (Seizure = 1; |
| Animal | | Weight | | Dose | Temp | Temp | Temp | Change/ | | No |
| ID | Sex | (g) | Treatment | (mg/kg) | (° C.) | (° C.) | Change | min | Observations | Seizure = 0) |
| 35 | F | 7.4 | Vehicle | 0 | 34.2 | 40 | 5.8 | 0.53 | Tonic-clonic, squeek | 1 |
| 39 | F | 7.6 | Vehicle | 0 | 35.6 | 40.3 | 4.7 | 0.78 | Tonic-clonic, squeek | 1 |
| 42 | F | 7.8 | Vehicle | 0 | 34.3 | 40.5 | 6.2 | 0.56 | Tonic-clonic, squeek | 1 |
| 46 | F | 7.8 | Vehicle | 0 | 35.7 | 40.9 | 5.2 | 0.52 | Tonic-clonic, squeek | 1 |
| 53 | F | 6.4 | Vehicle | 0 | 36.5 | 40.6 | 4.1 | 1.03 | Tonic-clonic seizure | 1 |
| 63 | F | 9 | Vehicle | 0 | 35.2 | 40 | 4.8 | 0.53 | Tonic-clonic, squeek | 1 |
| 67 | F | 9.6 | Vehicle | 0 | 35.9 | 40.4 | 4.5 | 0.50 | Tonic-clonic seizure, squeek | 1 |
| 71 | F | 10.3 | Vehicle | 0 | 35.4 | 40 | 4.6 | 0.77 | Tonic-clonic seizure, squeek | 1 |
| 113 | F | 7.9 | Vehicle | 0 | 35 | 40 | 5 | 0.45 | Tonic-clonic seizure, squeek | 1 |
| 179 | F | 7 | Vehicle | 0 | 35.5 | 40.4 | 4.9 | 0.61 | tonic-clonic, squeek | 1 |
| 295 | F | 7.9 | Vehicle | 0 | 35.5 | 40.2 | 4.7 | 0.52 | Tonic-clonic, squeek | 1 |
| 18 | M | 6.6 | Vehicle | 0 | 34.3 | 40.8 | 6.5 | 0.43 | Tonic-clonic | 1 |
| 31 | M | 7.4 | Vehicle | 0 | 34.4 | 40 | 5.6 | 0.51 | Tonic-clonic, squeek | 1 |
| 37 | M | 6.1 | Vehicle | 0 | 34.4 | 40.8 | 6.4 | 0.91 | Tonic-clonic, squeek, HLE | 1 |
| 41 | M | 7.3 | Vehicle | 0 | 35.6 | 38.6 | 3 | 0.60 | Tonic-clonic, squeek, HLE | 1 |
| 65 | M | 9.8 | Vehicle | 0 | 35.5 | 40.5 | 5 | 1.00 | Tonic-clonic seizure, squeek | 1 |
| 70 | M | 10.4 | Vehicle | 0 | 35.7 | 41.8 | 6.1 | 0.68 | Tonic-clonic | 1 |
| 75 | M | 8.2 | Vehicle | 0 | 36 | 40 | 4 | 0.40 | Tonic-clonic seizure, squeek | 1 |
| 93 | M | 9.3 | Vehicle | 0 | 35.4 | 39.9 | 4.5 | 0.45 | Tonic-clonic seizure, squeek | 1 |
| 129 | M | 7.3 | Vehicle | 0 | 35.9 | 40.7 | 4.8 | 0.40 | Tonic, clonic, squeek, HLE | 1 |
| 155 | M | 8.1 | Vehicle | 0 | NR | 40.7 | NR | NR | tonic-clonic, squeek | 1 |
| 219 | M | 7.7 | Vehicle | 0 | 35.6 | 40.6 | 5 | 0.38 | Tonic-clonic, squeek | 1 |
| | | | | | | | | | Total With No Seizure | 0% |

TABLE 2
Clobazam 10 mg/kg (30 minute pretreatment)

| | | | | | | | | | | GTCS |
|---|---|---|---|---|---|---|---|---|---|---|
| | | | | | Base | End | | Temp | | (Seizure = 1; |
| Animal | | Weight | | Dose | Temp | Temp | Temp | Change/ | | No |
| ID | Sex | (g) | Treatment | (mg/kg) | (° C.) | (° C.) | Change | min | Observations | Seizure = 0) |
| 10 | F | 8.3 | Clobazam | 10 | 33.3 | 42.7 | 9.4 | 0.31 | Tonic-clonic | 1 |
| 23 | F | 6.8 | Clobazam | 10 | 33.4 | 42.9 | 9.5 | 0.53 | Tonic-clonic, HLE | 1 |
| 25 | F | 6.7 | Clobazam | 10 | 33 | 42.3 | 9.3 | 0.58 | Tonic-clonic | 1 |
| 33 | F | 7.8 | Clobazam | 10 | 33.8 | 42 | 8.2 | 0.46 | Tonic-clonic | 1 |
| 44 | F | 7.7 | Clobazam | 10 | 35.8 | 42.2 | 6.4 | 0.58 | Tonic-clonic, squeek | 1 |
| 51 | F | 7.1 | Clobazam | 10 | 35.6 | 42.7 | 7.1 | 0.55 | Seizure free, 3 min hold | 0 |
| 64 | F | 10 | Clobazam | 10 | 35.5 | 42.6 | 7.1 | 0.51 | Seizure free, 3 min hold | 0 |
| 68 | F | 9.5 | Clobazam | 10 | 35.8 | 42.5 | 6.7 | 0.52 | Tonic-clonic | 1 |
| 106 | F | 7.8 | Clobazam | 10 | 34.6 | 42.6 | 8 | 0.47 | Seizure free, 3 min hold | 0 |
| 193 | F | 7.5 | Clobazam | 10 | 35.2 | 42.6 | 7.4 | 0.53 | seizure free, 3 min hold | 0 |
| 193 | F | 7.5 | Clobazam | 10 | 35.2 | 42.6 | 7.4 | 0.53 | seizure free, 3 min hold | 0 |
| 310 | F | 7.6 | Clobazam | 10 | 35.5 | 42.5 | 7 | 0.58 | Tonic-clonic on the onset | 1 |
| | | | | | | | | | of 3 min hold | |
| 15 | M | 7.3 | Clobazam | 10 | 33.7 | 41.9 | 8.2 | 0.59 | Tonic-clonic | 1 |
| 16 | M | 7.4 | Clobazam | 10 | 34.1 | 42.6 | 8.5 | 0.61 | Tonic-clonic | 1 |
| 36 | M | 8.1 | Clobazam | 10 | 35.3 | 42.5 | 7.2 | 0.51 | Seizure free, 3 min hold | 0 |
| 45 | M | 7.6 | Clobazam | 10 | 35.4 | 42.9 | 7.5 | 0.58 | Seizure free, 3 min hold | 0 |
| 48 | M | 9.4 | Clobazam | 10 | 34.9 | 42.8 | 7.9 | 0.53 | Seizure free, 3 min hold | 0 |
| 72 | M | 7.3 | Clobazam | 10 | 34.7 | 41.8 | 7.1 | 0.47 | Tonic-clonic seizure | 1 |
| 95 | M | 9.2 | Clobazam | 10 | 35.5 | 42 | 6.5 | 0.46 | Tonic-clonic seizure, | 1 |
| | | | | | | | | | squeek | |
| 104 | M | 7.3 | Clobazam | 10 | 35.3 | 42.5 | 7.2 | 0.65 | Tonic-clonic seizure | 1 |
| 138 | M | 8.2 | Clobazam | 10 | 36.1 | 41.7 | 5.6 | 0.80 | Tonic-clonic, squeek | 1 |
| 146 | M | 7.2 | Clobazam | 10 | 34.7 | 42.6 | 7.9 | 0.46 | seizure free, 3 min hold | 0 |
| 168 | M | 7.4 | Clobazam | 10 | 34.3 | 42.1 | 7.8 | 0.46 | Tonic-clonic | 1 |
| 288 | M | 8.7 | Clobazam | 10 | 34.9 | 42.5 | 7.6 | 0.54 | Tonic-clonic on the onset | 1 |
| | | | | | | | | | of 3 min hold | |
| | | | | | | | | | Total With No Seizure | 38% |

TABLE 3
Compound 1 0.1 mg/kg (60 minute pretreatment)

| | | | | | | | | | | GTCS |
|---|---|---|---|---|---|---|---|---|---|---|
| | | | | | Base. | End | | Temp | | (Seizure = 1; |
| Animal | | Weight | | Dose | Temp | Temp | Temp | Change/ | | No |
| ID | Sex | (g) | Treatment | (mg/kg) | (° C.) | (° C.) | Change | min | Observations | Seizure = 0) |
| 131 | F | 7.6 | Compound 1 | 0.1 | 35 | 41.4 | 6.4 | 0.40 | Tonic-clonic, squeek | 1 |
| 132 | F | 7.7 | Compound 1 | 0.1 | 35.3 | 41.2 | 5.9 | 0.45 | Tonic-clonic, squeek | 1 |
| 147 | F | 6.8 | Compound 1 | 0.1 | 34.5 | 41.9 | 7.4 | 0.57 | Tonic-clonic | 1 |
| 159 | F | 5.5 | Compound 1 | 0.1 | 34.5 | 41.8 | 7.3 | 0.61 | Tonic-clonic | 1 |
| 180 | F | 6.8 | Compound 1 | 0.1 | 36.3 | 41.6 | 5.3 | 0.66 | Tonic-clonic, squeek | 1 |
| 190 | F | 7.8 | Compound 1 | 0.1 | 34.6 | 42 | 7.4 | 0.49 | Tonic, clonic | 1 |
| 283 | F | 9.40 | Compound 1 | 0.1 | 35.9 | 41.7 | 5.8 | 0.36 | Tonic-clonic | 1 |
| 303 | F | 7.7 | Compound 1 | 0.1 | 34.2 | 42.8 | 8.6 | 0.51 | Seizure free, 3 min hold | 0 |
| 122 | M | 9.1 | Compound 1 | 0.1 | 35.9 | 42.6 | 6.7 | 0.52 | Tonic-clonic, seizure | 1 |
| | | | | | | | | | early into 3 min hold | |
| 145 | M | 6.9 | Compound 1 | 0.1 | 34.1 | 41.8 | 7.7 | 0.64 | Tonic-clonic | 1 |
| 154 | M | 8.3 | Compound 1 | 0.1 | 35.9 | 40.8 | 4.9 | 0.41 | Tonic-clonic | 1 |
| 167 | M | 7.6 | Compound 1 | 0.1 | 36.6 | 42 | 5.4 | 0.54 | Tonic-clonic, squeek | 1 |
| 170 | M | 8.1 | Compound 1 | 0.1 | 35.4 | 42.1 | 6.7 | 0.67 | Tonic-clonic | 1 |
| 206 | M | 6.4 | Compound 1 | 0.1 | 34.3 | 41.5 | 7.2 | 0.33 | Tonic-clonic | 1 |
| 211 | M | 5.9 | Compound 1 | 0.1 | 35.6 | 42.2 | 6.6 | 0.83 | Tonic-clonic | 1 |
| 305 | M | 7.6 | Compound 1 | 0.1 | 34.3 | 41.6 | 7.3 | 0.37 | Tonic-clonic | 1 |
| | | | | | | | | | Total With No Seizure | 6% |

TABLE 4
Compound 1 0.3 mg/kg (60 minute pretreatment)

| | | | | | | | | | GTCS |
|---|---|---|---|---|---|---|---|---|---|
| | | | | Base. | End | | | | (Seizure = 1; |
| Animal | | Weight | | Temp | Temp | Temp | Temp | | No |
| ID | Sex | (g) | Treatment | (° C.) | (° C.) | Change | Change/min | Observations | Seizure = 0) |
| 118 | F | 9.7 | Compound 1 | 35.2 | 42.7 | 7.5 | 0.34 | Tonic-clonic, squeek, | 1 |
| | | | | | | | | started 3 min hold, | |
| | | | | | | | | but died in beaker | |
| 125 | F | 7.6 | Compound 1 | 35.6 | 41.7 | 6.1 | 0.51 | Tonic-clonic | 1 |
| 136 | F | 8.2 | Compound 1 | NR | 41.4 | NR | NR | Tobic-clonic, squeek, HLE | 1 |
| 149 | F | 8.1 | Compound 1 | 35.3 | 42.3 | 7 | 0.50 | Tonic-clonic, squeek | 1 |
| 157 | F | 5.2 | Compound 1 | 33.8 | 42.5 | 8.7 | 0.36 | Seizure free, 3 min hold | 0 |
| 181 | F | 6.4 | Compound 1 | 36.1 | 42.3 | 6.2 | 0.41 | tonic-clonic, squeek, HLE | 1 |
| 225 | F | 7.8 | Compound 1 | 33.1 | 42.8 | 9.7 | 0.42 | Seizure free, 3 min hold | 0 |
| 262 | F | 9.30 | Compound 1 | 36 | 42.1 | 6.1 | 0.51 | Tonic-clonic | 1 |
| 302 | F | 6.4 | Compound 1 | 35.6 | 42.2 | 6.6 | 0.41 | Tonic-clonic | 1 |
| 137 | M | 8.7 | Compound 1 | 36.2 | 41.4 | 5.2 | 0.58 | Tonic,-clonic, squeek | 1 |
| 144 | M | 6.7 | Compound 1 | 34.1 | 42.4 | 8.3 | 0.92 | Tonic-clonic, squeek | 1 |
| 173 | M | 7.1 | Compound 1 | 36.1 | 42.8 | 6.7 | 0.56 | Seizure free, 3 min hold | 0 |
| 174 | M | 7.1 | Compound 1 | 34.9 | 41.9 | 7 | 0.64 | Tonic-clonic, squeek | 1 |
| 199 | M | 7.8 | Compound 1 | 35.8 | 42.2 | 6.4 | 0.46 | Tonic-clonic | 1 |
| 233 | M | 7.5 | Compound 1 | 35.7 | 42.4 | 6.7 | 0.45 | Tonic-clonic | 1 |
| 235 | M | 5.5 | Compound 1 | 34.7 | 42.6 | 7.9 | 0.32 | Seizure free, 3 min hold | 0 |
| 248 | M | 9.4 | Compound 1 | 35.4 | 42.6 | 7.2 | 0.34 | Seizure free, 3 min hold | 0 |
| 251 | M | 9.4 | Compound 1 | 34.8 | 43 | 8.2 | 0.48 | Seizure free, 3 min hold | 0 |
| | | | | | | | | Total With No Seizure | 33% |

TABLE 5
Compound 1 1.0 mg/kg 60 minute pretreatment)

| | | | | | | | | | | GTCS |
|---|---|---|---|---|---|---|---|---|---|---|
| | | | | | Base. | End | | Temp | | (Seizure = 1; |
| Animal | | Weight | | Dose | Temp | Temp | Temp | Change/ | | No |
| ID | Sex | (g) | Treatment | (mg/kg) | (° C.) | (° C.) | Change | min | Observations | Seizure = 0) |
| 79 | F | 7.2 | Compound 1 | 1 | 34.9 | 42.5 | 7.6 | 0.33 | Seizure free, 3 min hold | 0 |
| 88 | F | 9.4 | Compound 1 | 1 | 35.6 | 42.1 | 6.5 | 0.46 | Tonic-clonic seizure, | 1 |
| | | | | | | | | | squeek | |
| 92 | F | 9.2 | Compound 1 | 1 | 35.7 | 42.2 | 6.5 | 0.54 | Tonic-clonic seizure, | 1 |
| | | | | | | | | | squeek | |
| 94 | F | 9.4 | Compound 1 | 1 | 35.5 | 42.8 | 7.3 | 0.29 | seizure free, 3 min hold | 0 |
| 101 | F | 7.1 | Compound 1 | 1 | 36.2 | 42.4 | 6.2 | 0.39 | Tonic-clonic seizure, | 1 |
| | | | | | | | | | squeek | |
| 102 | F | 7.2 | Compound 1 | 1 | 34.5 | 42.5 | 8 | 0.50 | Seizure free, 3 min hold | 0 |
| 103 | F | 7.2 | Compound 1 | 1 | 35.4 | 42.2 | 6.8 | 0.43 | Tonic-clonic seizure, | 1 |
| | | | | | | | | | squeek | |
| 112 | F | 7.6 | Compound 1 | 1 | 36.1 | 42.8 | 6.7 | 0.74 | Tonic-clonic seizure | 1 |
| | | | | | | | | | during half way into 3 | |
| | | | | | | | | | min hold | |
| 114 | F | 9 | Compound 1 | 1 | NR | 42.3 | NR | NR | Tonic-clonic seizure, | 1 |
| | | | | | | | | | squeek | |
| 151 | F | 7.9 | Compound 1 | 1 | 35.2 | 42.9 | 7.7 | 0.55 | Seizure free, 3 min hold | 0 |
| 269 | F | 7.8 | Compound 1 | 1 | 36.3 | 42.5 | 6.2 | 0.27 | Seizure free, 3 min hold | 0 |
| 78 | M | 8.4 | Compound 1 | 1 | 34.9 | 41.9 | 7 | 0.50 | Tonic-clonic seizure, | 1 |
| | | | | | | | | | squeek | |
| 81 | M | 7 | Compound 1 | 1 | 35 | 42.5 | 7.5 | 0.47 | Seizure free, 3 min hold | 0 |
| 83 | M | 7.3 | Compound 1 | 1 | 34.5 | 42.9 | 8.4 | 0.53 | Seizure free, 3 min hold | 0 |
| 91 | M | 8.6 | Compound 1 | 1 | 35.8 | 42.5 | 6.7 | 0.61 | Tonic-clonic seizure, | 1 |
| | | | | | | | | | squeek | |
| 109 | M | 8 | Compound 1 | 1 | 35.3 | 42.3 | 7 | 0.54 | Tonic-clonic seizure, | 1 |
| | | | | | | | | | squeek | |
| 111 | M | 7.8 | Compound 1 | 1 | 35.5 | 42.2 | 6.7 | 0.42 | Tonic-clonic seizure | 1 |
| 115 | M | 9.5 | Compound 1 | 1 | 34.8 | 42.8 | 8 | 0.57 | Tonic-clonic seizure in | 1 |
| | | | | | | | | | the beginning of 3 min | |
| | | | | | | | | | hold, HLE | |
| 128 | M | 7.7 | Compound 1 | 1 | 35.3 | 41.5 | 6.2 | 0.39 | Tonic-clonic seizure | 1 |
| 135 | M | 8.9 | Compound 1 | 1 | 35.2 | 41.3 | 6.1 | 0.36 | Tonic-clonic, Squeek | 1 |
| 143 | M | 6.8 | Compound 1 | 1 | 34.4 | 42.6 | 8.2 | 0.39 | seizure free, 3 min hold | 0 |
| 189 | M | 6.5 | Compound 1 | 1 | 36 | 42.8 | 6.8 | 0.40 | Tonic clonic seizure in | 1 |
| | | | | | | | | | the mddle of 3 min hold | |
| | | | | | | | | | Total With No Seizure | 36% |

TABLE 6
Compound 2 3 mg/k g (30 minute pretreatment)

| | | | | | | | | | | GTCS |
|---|---|---|---|---|---|---|---|---|---|---|
| | | | | | Base. | End | | Temp | | (Seizure = 1; |
| Animal | | Weight | | Dose | Temp | Temp | Temp | Change/ | | No |
| ID | Sex | (g) | Treatment | (mg/kg) | (° C.) | (° C.) | Change | min | Observations | Seizure = 0) |
| 223 | F | 7.8 | Compound 2 | 3 | 34.8 | 41.4 | 6.6 | 0.55 | Tonic-clonic | 1 |
| 238 | F | 7.7 | Compound 2 | 3 | 35.3 | 41.7 | 6.4 | 0.32 | Tonic-clonic | 1 |
| 241 | F | 7.6 | Compound 2 | 3 | 34.2 | 42.6 | 8.4 | 0.47 | Seizure free, 3 min hold | 0 |
| 250 | F | 9.1 | Compound 2 | 3 | 34.9 | 41.8 | 6.9 | 0.49 | Tonic-clonic | 1 |
| 252 | F | 8.8 | Compound 2 | 3 | 34.6 | 41.7 | 7.1 | 0.42 | Tonic-clonic, squeek | 1 |
| 278 | F | 10.50 | Compound 2 | 3 | 35 | 42.2 | 7.2 | 0.36 | Tonic-clonic | 1 |
| 268 | F | 7.80 | Compound 2 | 3 | 36.4 | 42.4 | 6 | 0.43 | Tonic-clonic | 1 |
| 229 | M | 7.8 | Compound 2 | 3 | 35.9 | 41.3 | 5.4 | 0.42 | Tonic-clonic | 1 |
| 231 | M | 7.4 | Compound 2 | 3 | 35.9 | 41.7 | 5.8 | 0.53 | Tonic-clonic | 1 |
| 239 | M | 7.4 | Compound 2 | 3 | 35.1 | 41.3 | 6.2 | 0.41 | Tonic-clonic, squeek | 1 |
| 292 | M | 7.60 | Compound 2 | 3 | 35.5 | 42.1 | 6.6 | 0.51 | Tonic-clonic | 1 |
| 255 | M | 6.8 | Compound 2 | 3 | 35.7 | 41.8 | 6.1 | 0.47 | Tonic-clonic, squeek | 1 |
| 259 | M | 9.30 | Compound 2 | 3 | 35.2 | 42 | 6.8 | 0.27 | Tonic-clonic | 1 |
| 304 | M | 7.1 | Compound 2 | 3 | 35.4 | 42.5 | 7.1 | 0.71 | Tonic-clonic on the onset | 1 |
| | | | | | | | | | of 3 min hold | |
| | | | | | | | | | Total With No Seizure | 7% |

TABLE 7
Compound 2 10 mg/kg (30 minute pretreatment)

| | | | | | | | | | | GTCS |
|---|---|---|---|---|---|---|---|---|---|---|
| | | | | | Base. | End | | Temp | | (Seizure = 1; |
| Animal | | Weight | | Dose | Temp | Temp | Temp | Change/ | | No |
| ID | Sex | (g) | Treatment | (mg/kg) | (° C.) | (° C.) | Change | min | Observations | Seizure = 0) |
| 169 | F | 7.5 | Compound 2 | 10 | 35.4 | 42.8 | 7.4 | 0.53 | Seizure free, 3 min hold | 0 |
| 182 | F | 6.8 | Compound 2 | 10 | 35.6 | 42.5 | 6.9 | 0.41 | tonic-clonic on the onset | 1 |
| | | | | | | | | | of 3 min hold | |
| 215 | F | 5.8 | Compound 2 | 10 | 34.4 | 42.7 | 8.3 | 0.55 | Seizure free, 3 min hold | 0 |
| 247 | F | 9 | Compound 2 | 10 | 35.7 | 42.8 | 7.1 | 0.44 | Seizure free, 3 min hold | 0 |
| 254 | F | 7.2 | Compound 2 | 10 | 34.8 | 42.7 | 7.9 | 0.44 | Seizure free, 3 min hold | 0 |
| 296 | F | 7.90 | Compound 2 | 10 | 35.6 | 42.6 | 7 | 0.50 | Tonic-clonic seizure half | 1 |
| | | | | | | | | | way into 3 min hold | |
| 307 | F | 6.8 | Compound 2 | 10 | 35.3 | 42.1 | 6.8 | 0.52 | Tonic-clonic | 1 |
| 184 | M | 6.1 | Compound 2 | 10 | 35.4 | 42.5 | 7.1 | 0.55 | tonic-clonic on the onset | 1 |
| | | | | | | | | | of 3 min hold | |
| 202 | M | 7.8 | Compound 2 | 10 | 34.6 | 42.8 | 8.2 | 0.41 | Seizure free, 3 min hold | 0 |
| 209 | M | 6.2 | Compound 2 | 10 | 35.2 | 42 | 6.8 | 0.43 | Tonic-clonic | 1 |
| 234 | M | 7.2 | Compound 2 | 10 | 35.9 | 42.5 | 6.6 | 0.51 | Tonic-clonic seizure on | 1 |
| | | | | | | | | | the onset | |
| | | | | | | | | | of the 3 min hold | |
| 240 | M | 7.7 | Compound 2 | 10 | 35.7 | 40 | 4.3 | 0.39 | Tonic-clonic, squeek | 1 |
| 286 | M | 9.60 | Compound 2 | 10 | 34.9 | 42.7 | 7.8 | 0.41 | Seizure free, 3 min hold | 0 |
| 260 | M | 9.10 | Compound 2 | 10 | NR | 42.5 | NR | | Seizure free, 3 min hold | 0 |
| | | | | | | | | | Total With No Seizure | 50% |

TABLE 8
Compound 2 30 mg/kg (30 minute pretreatment)

| | | | | | | | | | | GTCS |
|---|---|---|---|---|---|---|---|---|---|---|
| | | | | | Base. | End | | Temp | | (Seizure = 1; |
| Animal | | Weight | | Dose | Temp | Temp | Temp | Change/ | | No |
| ID | Sex | (g) | Treatment | (mg/kg) | (° C.) | (° C.) | Change | min | Observations | Seizure = 0) |
| 183 | F | 6.5 | Compound 2 | 30 | 36 | 42.6 | 6.6 | 0.39 | seizure free, 3 min hold | 0 |
| 203 | F | 7.4 | Compound 2 | 30 | 35.3 | 42.7 | 7.4 | 0.35 | Seizure free, 3 min hold | 0 |
| 208 | F | 6 | Compound 2 | 30 | 33.4 | 42.5 | 9.1 | 0.51 | Seizure free, 3 min hold | 0 |
| 237 | F | 11.9 | Compound 2 | 30 | 36.2 | 41.5 | 5.3 | 0.48 | Tonic-clonic seizure | 1 |
| 290 | F | 9.40 | Compound 2 | 30 | 36.5 | 42.8 | 6.3 | 0.48 | Seizure free, 3 min hold | 0 |
| 265 | F | 9.50 | Compound 2 | 30 | 36.5 | 42.7 | 6.2 | 0.41 | Seizure free, 3 min hold | 0 |
| 301 | F | 7.9 | Compound 2 | 30 | 34.8 | 42.7 | 7.9 | 0.46 | Tonic-clonic | 1 |
| 306 | F | 7.2 | Compound 2 | 30 | 35.6 | 42.8 | 7.2 | 0.55 | Seizure free, 3 min hold | 0 |
| 176 | M | 7.7 | Compound 2 | 30 | 35.2 | 42.5 | 7.3 | 0.66 | Tonic-clonic, squeek | 1 |
| 186 | M | 6.8 | Compound 2 | 30 | 36 | 42.8 | 6.8 | 0.49 | seizure free, 3 min hold | 0 |
| 196 | M | 7.7 | Compound 2 | 30 | 34.8 | 42.6 | 7.8 | 0.41 | Seizure free, 3 min hold | 0 |
| 218 | M | 7.8 | Compound 2 | 30 | 35.5 | 43 | 7.5 | 0.54 | Seizure free, 3 min hold | 0 |
| 249 | M | 9.2 | Compound 2 | 30 | 33.1 | 42.7 | 9.6 | 0.53 | Seizure free, 3 min hold | 0 |
| 294 | M | 7.90 | Compound 2 | 30 | 35.7 | 42.7 | 7 | 0.50 | Seizure free, 3 min hold | 0 |
| 272 | M | 7.70 | Compound 2 | 30 | 34.1 | 41.6 | 7.5 | 0.63 | Tonic-clonic | 1 |
| | | | | | | | | | Total With No Seizure | 73% |

TABLE 9
Compound 2 100 mg/kg (30 minute pretreatment)

| | | | | | Base. | End | | Temp | | |
|---|---|---|---|---|---|---|---|---|---|---|
| Animal | | Weight | | Dose | Temp | Temp | Temp | Change/ | | |
| ID | Sex | (g) | Treatment | (mg/kg) | (° C.) | (° C.) | Change | min | Observations | GTCS |
| 171 | F | 7.3 | Compound 2 | 100 | 35.3 | 42.7 | 7.4 | 0.53 | Seizure free, 3 min hold | 0 |
| 212 | F | 5.7 | Compound 2 | 100 | 34.8 | 42.5 | 7.7 | 0.33 | Seizure free, 3 min hold | 0 |
| 214 | F | 6.3 | Compound 2 | 100 | 34.2 | 42.7 | 8.5 | 0.50 | Seizure free, 3 min hold | 0 |
| 216 | F | 7.3 | Compound 2 | 100 | 34.7 | 42.9 | 8.2 | 0.59 | Seizure free, 3 min hold | 0 |
| 178 | M | 6.7 | Compound 2 | 100 | 35.2 | 42.6 | 7.4 | 0.31 | seizure free, 3 min hold | 0 |
| 188 | M | 6.7 | Compound 2 | 100 | 35.4 | 42.6 | 7.2 | 0.36 | seizure free, 3 min hold | 0 |
| 207 | M | 5.5 | Compound 2 | 100 | 33 | 42.7 | 9.7 | 0.54 | Seizure free, 3 min hold | 0 |
| 236 | M | 11.9 | Compound 2 | 100 | 34.8 | 42.5 | 7.7 | 0.29 | Seizure free, 3 min hold | 0 |
| | | | | | | | | | Total With No Seizure | 100% |

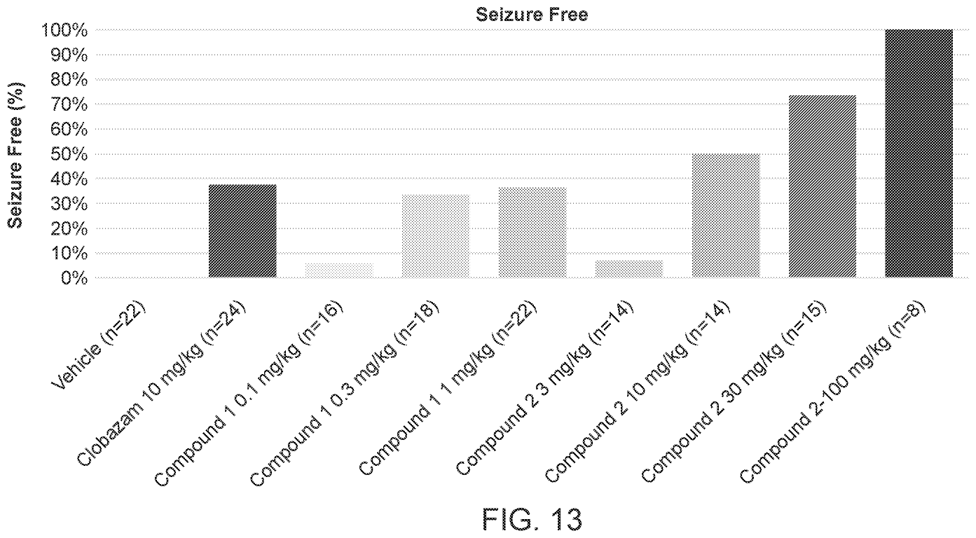

A summary of the data is presented in Table 10 and FIG. 13.

TABLE 10
Summary of Seizure Data

| | | End | | | |
|---|---|---|---|---|---|
| | | Temper- | | Temp | |
| | Base. | ature | Temp | Change/ | Seizure |
| | Temp (° C.) | (° C.) | Change | min | free % |
| Vehicle (n = 22) | 35.31 | 40.35 | 5.02 | 0.60 | 0% |
| Clobazam- | 34.82 | 42.42 | 7.60 | 0.53 | 38% |
| 10 mg/kg (n = 24) | | | | | |
| Compound 1- | 35.15 | 41.81 | 6.66 | 0.52 | 6% |
| 0.1 mg/kg | | | | | |
| (n = 16) | | | | | |
| Compound 1- | 35.20 | 42.29 | 7.15 | 0.48 | 33% |
| 0.3 mg/kg | | | | | |
| (n = 18) | | | | | |
| Compound 1- | 35.34 | 42.39 | 7.05 | 0.46 | 36% |
| 1.0 mg/kg | | | | | |
| (n = 22) | | | | | |
| Compound 2- | 35.28 | 41.89 | 6.61 | 0.45 | 7% |
| 3 mg/kg (n = 14) | | | | | |
| Compound 2- | 35.27 | 42.37 | 7.09 | 0.47 | 50% |
| 10 mg/kg (n = 14) | | | | | |
| Compound 2- | 35.25 | 42.55 | 7.30 | 0.49 | 73% |
| 30 mg/kg (n = 15) | | | | | |
| Compound 2- | 34.60 | 42.60 | 8.00 | 0.37 | 100% |
| 100 mg/kg (n = 8) | | | | | |

As shown in Table 10, the GABA_{A} receptor modulators Compound 1 and Compound 2 reduced the amount of seizures in the animal model relative to administration of vehicle. Furthermore, Compound 1 and Compound 2 performed at least as well as the clobazam positive control. Given that clobazam is a first line treatment for seizures, GABA_{A} receptor modulators are promising therapeutics for treating seizure. Indeed, the data shows that Compound 2 treats seizures in a dose dependent fashion, with a 100 mg/kg dose completely preventing seizures in each animal (n=8).

Indeed, administration of from about 0.1 mg/kg to about 100 mg/kg of either Compound 1 or Compound 2 was shown to be effective at preventing seizures in the mouse model. A person of ordinary skill in the art would be able to extrapolate this effective amount to other animals. For example, using an allometric scaling factor for human of 12, an equivalent human dose would be approximately 0.008 mg/kg to about 10 mg/kg.

One of skill in the art would understand that the efficacy of Compound 1 and Compound 2 at treating seizures in the hyperthermia-induced animal model is not limited to the specific compounds. Rather, a person of ordinary skill in the art would reasonably expect that GABA_{A} receptor modulators as a class would be effective at treating seizures in a manner comparable to the efficacy seen with Compound 1 and Compound 2. Furthermore, the skilled artisan would understand that the efficacy would not be limited to preventing seizures in the Scn1a^{+/-} knockout mouse. Rather, the animal model is a well established animal model for various epileptic conditions, including Dravet Syndrome. Accordingly, the skilled artisan would reasonably expect that the entire class of GABA_{A} receptor modulators would be effective at treating epileptic conditions by preventing seizures.

Accordingly, the results demonstrate the potential of GABA_{A} receptor modulators, as a class, at treating epileptic conditions generally, as demonstrated in the mouse heat induced seizure model.

### Example 4—Pharmacokinetic Determination

In order to determine pharmacokinetic parameters for Compound 1 and Compound 2, P14-P16 mice (n=7 group⁻¹·sex⁻¹·timepoint⁻¹) is dosed with Compound 1 and Compound 2 and terminal blood samples will be collected at the following timepoints:
a. Compound 1: 30 min, 1 h, 2 h, 4 h, 8 h, 12 h, 24 h
b. Compound 2: 30 min, 1 h, 2 h, 4 h, 8 h, 12 h, 24 h

Blood collection: Immediately prior to blood collection, the mouse will be euthanized by C₀₂ inhalation. Blood will be collected via cardiac puncture using a 27 gauge needle and transferred into a 500 μL K₃EDTA tube and immediately placed into wet ice. The samples will be centrifuged at 2900 g for 10 minutes under refrigerated conditions (set to maintain +4° C.) within 2 hours of collection. The plasma will be transferred into individual tubes within 2 hours and store at −20° C.

Dose Formulation Concentration Verification

A 0.3 mL aliquot of each formulation will be collected from the middle dose concentration position and stored at 2-4° C. or lower in a sealed vial wrapped in parafilm until the end of the study.

A calibration curve consisting of at least 6 calibration standards will be constructed using an LC/UV method.

The concentrations of the test compound in the dose formulation samples will be determined by the LC/UV method.

Determination of Plasma Levels of the Test Item

An LC-MS/MS method for the quantitative determination of test compound in biological matrix will be developed under non-GLP compliance.

A calibration curve with at least 6 non-zero calibration standards (STDs) will be constructed for the LC-MS/MS method.

Assay of Study Samples

The study samples will be assayed in batches using LC-MS/MS after the appropriate preparation. In general, one set of calibration curve with two sets of QC samples consisting of low, middle and high concentrations will be applied if sample numbers are no more than 48. If sample numbers are more than 48, then two sets of calibration curves with two sets of QC samples will be applied.

In general, one reagent blank, one plasma blank, and if applicable, two plasma blanks containing only internal standard will be run.

Samples in the same matrix of different PK projects will be quantified in the same analysis run.

Acceptance Criteria of Analytical Batch

Linearity: ≥75% of STDs are back calculated to within ±20% of their nominal values (±25% for LLOQ) in biofluid and within 25% of their nominal values (30% for LLOQ) in tissue homogenate and feces samples.

Accuracy: ≥67% all QC samples are back calculated to within ±20% of their nominal values for biofluid and within 25% of their nominal values for tissue and feces samples.

Specificity: The mean calculated concentration of analyte in the single blank matrix should be less than 0.5 times of the LLOQ.

Pharmacokinetic Analysis

Analysis of Pharmacokinetic data will be performed for each dose level, gender and sampling occasion.

In general, the following toxicokinetic parameters will be calculated/measured if possible: C₀, Cₘₐₓ, Tₘₐₓ, T_{1/2}, AUC₀₋ₜ, AUC_{0-inf}, MRT₀₋₄, MRT_{0-inf}, Cl and Vss.

Values below the lower limit of quantification will be reported as BLQ in individual tables and considered as zero for descriptive statistics and integrated into the statistic calculations. When the mean of the individual concentrations is below the limit of quantification, BLQ will be reported.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 5—Evaluation of the Acute Anticonvulsant Efficacy of Compound 1 in Mouse and Rat Models of Acute Seizures

Methods:

Compound 1 was tested for anticonvulsant efficacy in the acute 6 Hz seizure model at 32 and 44 mA intensity in CF-1 mice. Compound 1 was evaluated for dose-related efficacy in a 6 Hz seizure test following acute administration at a single time point and compared to efficacy of the positive control diazepam (DZP) (tested 0.5 hours after drug administration). Formulation vehicle (VEH) was used as a negative control.

After oral administration of the compound, mice were challenged at the appropriate time point with a 32 or 44 mA current for 3 seconds delivered through corneal electrodes to elicit a typical seizure. The 6 Hz seizure was characterized by an initial momentary stun followed immediately by forelimb clonus, twitching of the vibrissae, and Straub tail 1. Animals not displaying all of these behaviors within the immediate (5-10 second) period post-stimulation were considered “protected” (N=number protected/F=number tested).

Compound 1 was administered and tested for anticonvulsant efficacy 3 hours later. The compound was administered at 3 doses (0.01, 0.03, and 0.1 mg/kg; n=8 male CF-1 mice/dose group), in a volume of 0.01 mL/g. The testing was conducted over the course of 1-2 days with animals in each testing cohort randomized to receive a single dose of the investigational compound or vehicle. For every day that testing was conducted, animals were tested in cohorts of 20-30 mice/session and treatment was randomized across all groups and testing days. The dose of compound required to produce the desired endpoint in 50% of animals (ED50) and the 95% confidence interval was calculated by a computer program based on the Probit method 6. A group of vehicle-treated and DZP-treated (1 mg/kg, P.O.) mice will also be tested in parallel at the previously determined time of peak effect of DZP (0.5 hrs); all testing was conducted by an investigator blinded to treatment condition. A statistically significant (P≤0.05) increase of the number of the protected mice over that of negative control was considered to have efficacy. A summary of results is provided in Table 11.

TABLE 11
Dose and efficacy of Compound 1 in 6 Hz seizure study in
mice 32 mA vs vehicle

| Dose | Statistically significant efficacy |
|---|---|
| (mg/kg) | observed (Chi-square test, P ≤ 0.05) |
| 0.01 | N |
| 0.03 | Y |
| 0.1 | Y |

1. A method of treating an epileptic condition in a subject, comprising administering to the subject a therapeutically effective amount of a compound of a general formula (1a), general formula (1b) or general formula (1c), 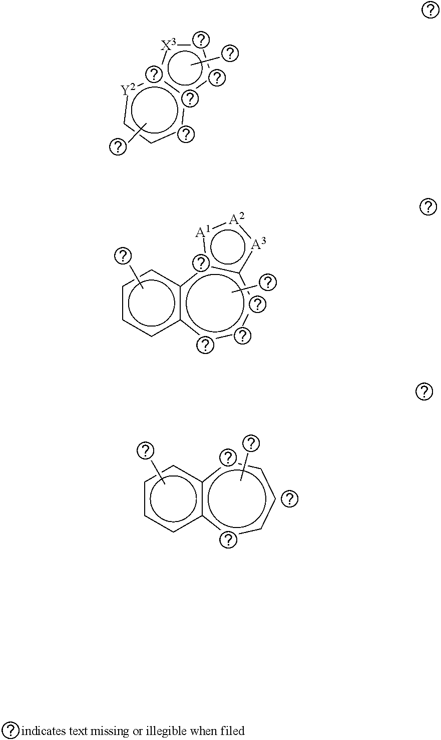
wherein
X¹, X², X³, X⁴ and X⁵ are independently —C, —N, —S or —O wherein at least two of X¹, X², X³, X⁴ and X⁵ are —N,
Y¹ and Y² are independently —C or —N,
m of R¹ₘ is 1, wherein R¹ is an unsubstituted phenyl, a phenyl substituted with C₁-C₄ alkyl, F, Cl, Br, I, —CN, a substituted or unsubstituted biphenyl or —(C═O)—R³, wherein R³ is a substituted or unsubstituted aryl or 5- to 6-membered heteroaryl,
n of R²ₙ is 1 or 2, wherein each R² is independently a substituted or unsubstituted C₃-C₈ cycloalkyl, a substituted or unsubstituted C₁-C₆ alkyl, a substituted or unsubstituted C₁-C₆ alcohol, a substituted or unsubstituted 6-membered heteroaryl, a halogen, or —O—CH₂—R⁴, wherein R⁴ is a substituted or unsubstituted 5- or 6-membered heteroaryl,
Z¹, Z³, Z⁴, and Z⁵ are independently —C, —N, —S or —O,
A¹, and A² and A³, are independently —C, —N, or —C(C═O)—O—R⁷, or 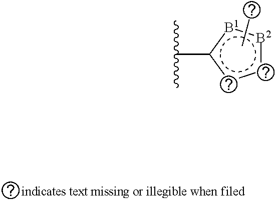
wherein
R⁷ is alkyl,
B¹, B², B³, and B⁴ are independently —C, —N, or —O, s of R²¹ₛ is 1, 2, 3 or 4, and
l of R⁵ₗ is 1 or 2, wherein each R⁵ is independently a C₁-C₄ alkynyl or a halogen,
k of R⁶ₖ is 1, 2, 3 or 4, wherein each R⁶ is independently a substituted or unsubstituted aryl, a substituted or unsubstituted heteroaryl, a substituted or unsubstituted C₁-C₃ alkyl, or hydrogen,
p of R¹²ₚ is 1 or 2, wherein each R¹² is independently a substituted or unsubstituted C₁-C₄ alkyl, I, Br, Cl or F, and
q of R¹³_{q} is 1, 2, 3 or 4, wherein each R¹³ is independently a substituted or unsubstituted aryl, a substituted or unsubstituted heteroaryl, a substituted or unsubstituted C₁-C₃ alkyl, oxygen or hydrogen, to treat the epileptic condition in the subject.

2. The method of claim 1, wherein the epileptic condition is epilepsy associated with a mutation in a sodium channel.

3. The method of claim 1, wherein the compound is of a general formula (2), (3), (4), (5), (1e) or (7),

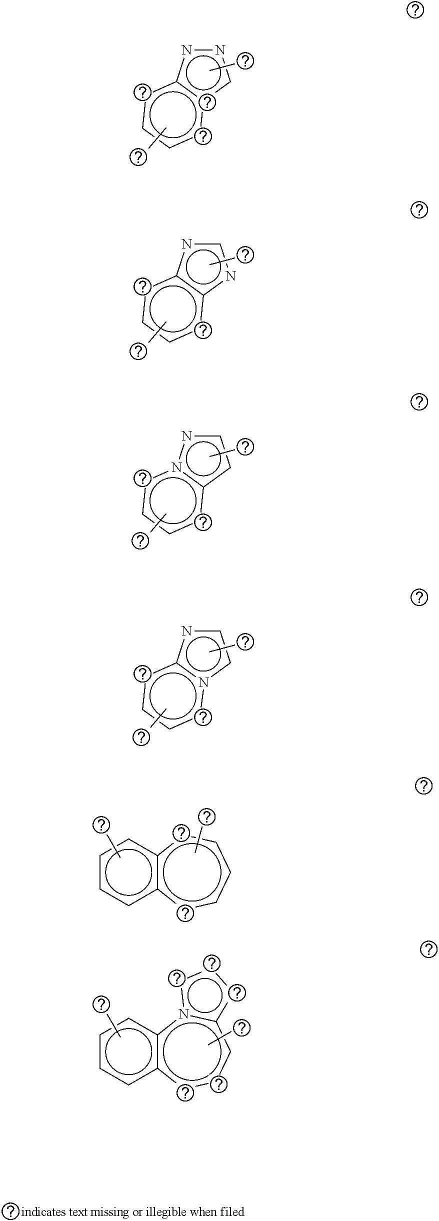

4. The method of claim 1, wherein the compound is of the general formula (2a), (3a), (4a), (5a), (5b), (1e) or (7a)

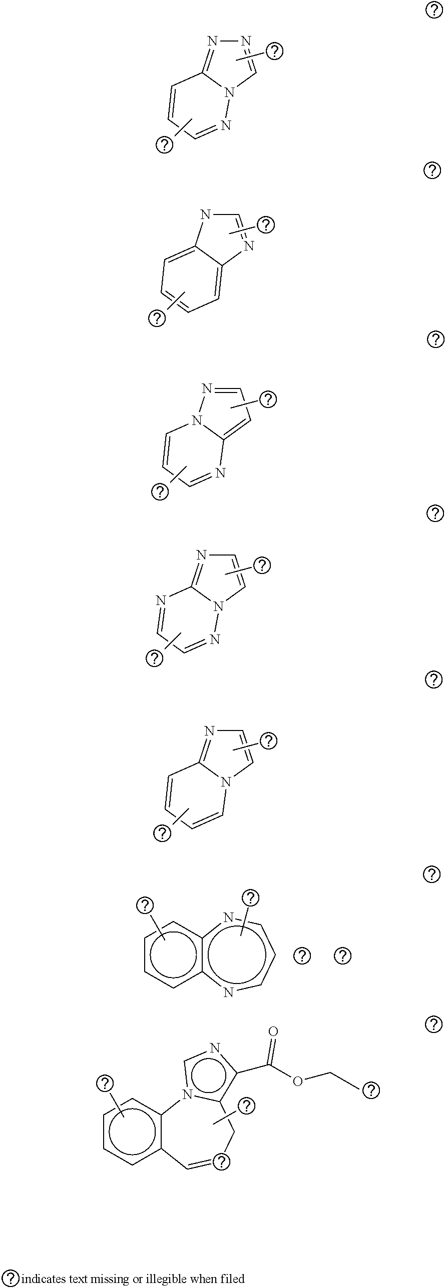

5. The method of claim 1, wherein
m of R¹ₘ is 1, and wherein R¹ is:
an unsubstituted phenyl,
a substituted phenyl comprising C₁-C₄-alkyl, F, Cl, Br, I, —CN as substituents,
an unsubstituted biphenyl,
a substituted biphenyl comprising at least one —CN as a substituent, a substituted biphenyl comprising at least one —CN as a substituent, or —(C═O)—R³, wherein R³ is pyridine.

6. The method of claim 1, wherein the compound is of the general formula (2a′), (3a′), (4a′), (5a′), (5b′), (VI) or (7a′), 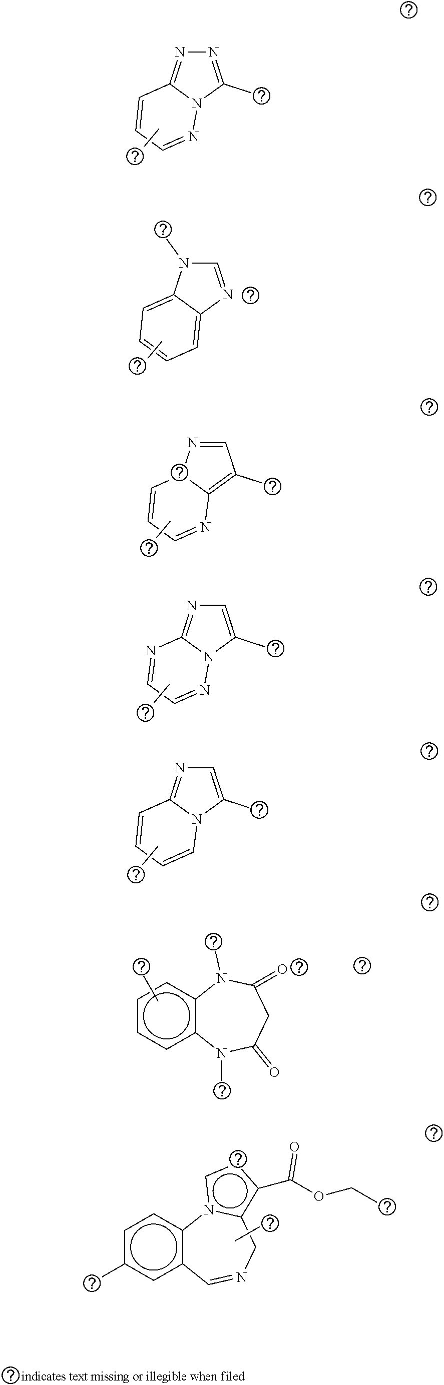
wherein
R¹⁰ is a substituted or unsubstituted aryl, a substituted or unsubstituted C₁-C₃ alkyl or hydrogen,
R¹¹ is a substituted or unsubstituted aryl, a substituted or unsubstituted C₁-C₃ alkyl, or hydrogen, and
p of R¹²ₚ is 1 and R¹² is I, Br, Cl or F.

7. The method of claim 1, wherein the compound is of the general formula (2a″), (3a″), (4a″), (5a″), (Sb″), (VIa″) or (7a″), 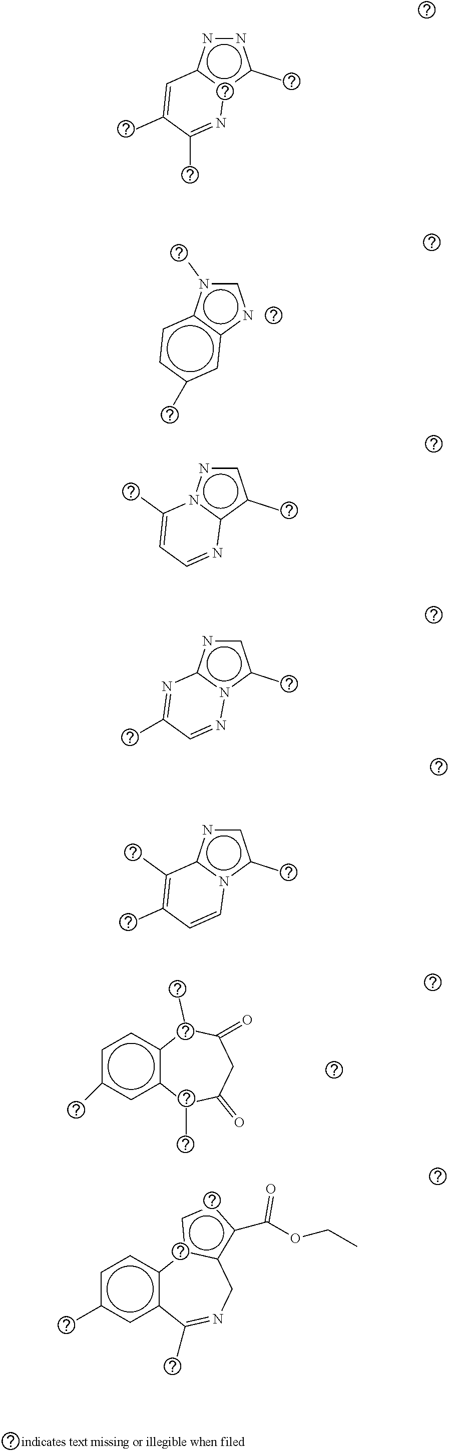
wherein R⁷ is:
an unsubstituted C₁-C₆ alkyl,
an unsubstituted C₃-C₈ cycloalkyl,
an unsubstituted C₁-C₆ alcohol,
R⁸ is:
O—CH₂—R⁴, wherein R⁴ is a substituted or unsubstituted 5-membered heteroaryl, or
an unsubstituted C₁-C₆ alcohol,
R⁹ is:
an unsubstituted C₆ heteroaryl, or
a halogen, or
R⁹ is an unsubstituted 6-membered heteroaryl in formula (4a″) or a halogen in formula (5b″),
R¹⁰ is a C₁-C₃ alkyl or hydrogen,
R¹¹ is a substituted or unsubstituted aryl or heteroaryl,
R¹⁴ is a substituted or unsubstituted aryl or heteroaryl,
R¹² is I, Cl, Br or F, or
R⁵ is C₂ alkynyl or I.

8. The method of claim 1, wherein the compound is an a1, a2, a3, or a5 GABAA receptor modulator or a positive, allosteric a2 or a3 GABAA receptor modulator.

9.-14. (canceled)

15. The method of claim 1, wherein the epileptic condition is selected from the following: Benign centrotemporal lobe epilepsy of childhood, Benign occipital epilepsy of childhood (BOEC), Autosomal dominant nocturnal frontal lobe epilepsy (ADNFLE), Primary reading epilepsy, Childhood absence epilepsy (CEA), Juvenile absence epilepsy, Juvenile myoclonic epilepsy (JME), Symptomatic localization-related epilepsies, Temporal lobe epilepsy (TLE), Frontal lobe epilepsy, Rasmussen's encephalitis, West syndrome, Dravet syndrome, Progressive myoclonic epilepsies, and Lennox-Gastaut syndrome (LGS).

16. (canceled)

17. A method of treating Dravet Syndrome in a subject comprising administering to a subject an amount of a pharmaceutical composition comprising a compound selected from the following: 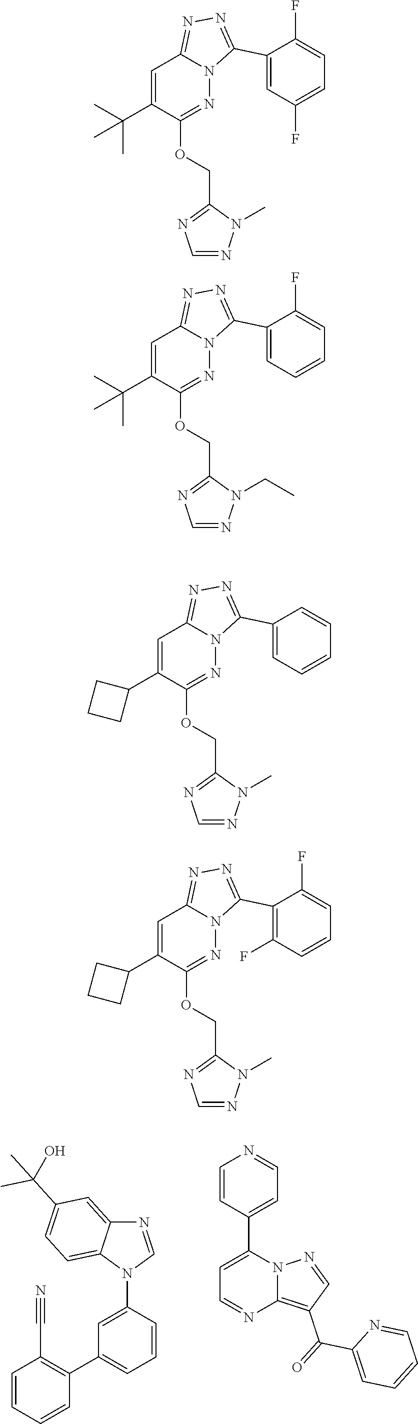 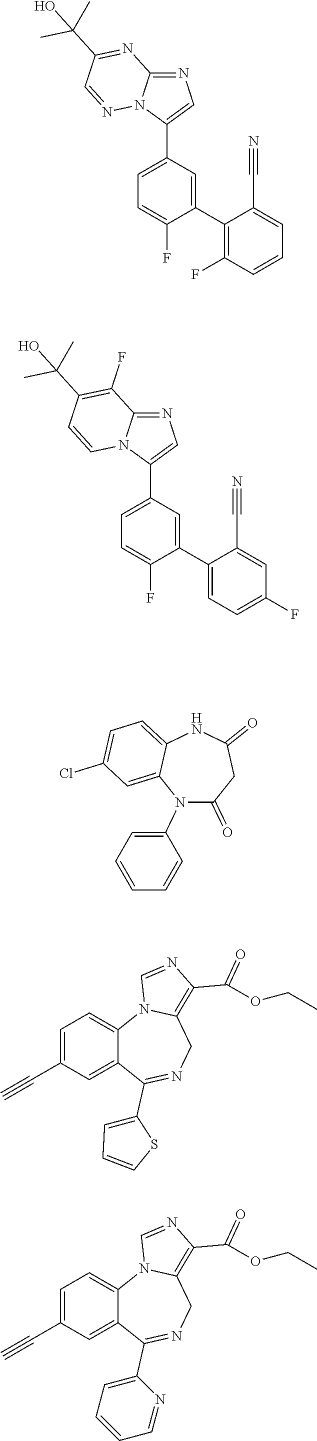 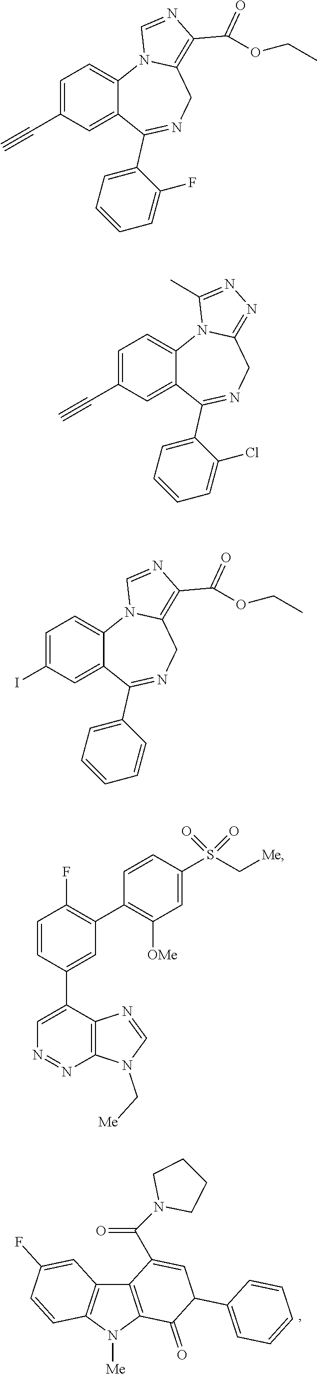 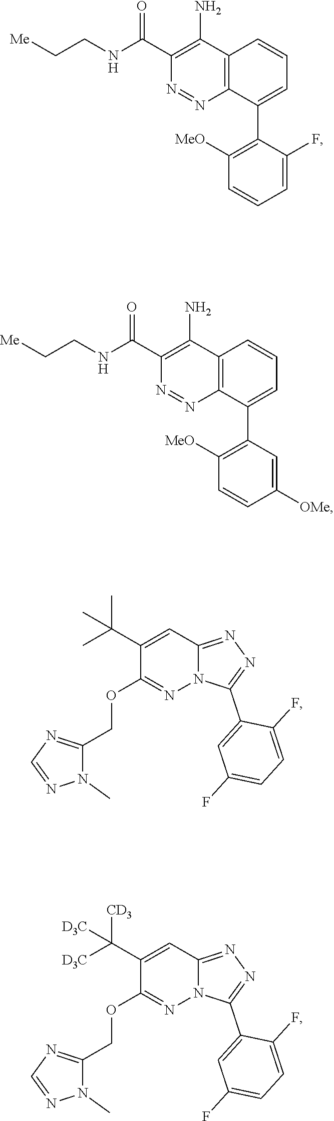 [SMILES: CCCNC(=O)c1nnc2c(-c3c(F)cccc3OC)cccc2c1N ; CCCNC(=O)c1nnc2c(-c3cc(OC)ccc3OC)cccc2c1N ; Cn1ncnc1COc1nn2c(-c3cc(F)ccc3F)nnc2cc1C(C)(C)C ; [2H]C([2H])([2H])C(c1cc2nnc(-c3cc(F)ccc3F)n2nc1OCc1ncnn1C)(C([2H])([2H])[2H])C([2H])([2H])[2H]]
a salt thereof, and a polymorph thereof, to treat the Dravet Syndrome in the subject.

18. (canceled)

19. The method of claim 17, wherein the amount is effective to treat the Dravet Syndrome when administered at a dose of from about 0.003 mg/kg per day to about 10 mg/kg per day of a body weight of the subject when administered to the subject.

20.-24. (canceled)

25. The method of claim 17, comprising the salt, wherein the salt is a phosphate salt or a sulfate salt.

26. (canceled)

27. The method of claim 17, comprising the polymorph, wherein the polymorph has an X-ray powder diffraction (XRPD) having characteristic peak locations of at least three of the values selected from the following: about 6.4, 7.5, 10.2, 12.7, 13.3, 14.5, 16.0, 17.1, 17.4, 17.9, 18.5, 19.1, 19.7, 20.3, 20.9, 21.5, 22.6, 23.7, 26.2, 26.7, 26.9, 27.5, 28.4, 30.2, and 32.1±0.2 degrees, 2-Theta, when measured using: (a) an X-ray wavelength parameter of Cu: K-Alpha (λ=1.54179 Å); (b) an X-Ray tube voltage setting of 40 kV and current of 40 mA; (c) a scan scope of from about 3 degrees to about 40 degrees; (d) a sample rotation speed of about 15 rpm; and (e) a scanning rate of 10 degrees per minute.

28. (canceled)

29. (canceled)

30. The method of claim 17, wherein the administering of the amount that is effective to treat Dravet Syndrome does not produce drowsiness or sedation in the subject.

31.-38. (canceled)

39. A method of treating an epileptic condition in a subject, comprising administering to the subject a pharmaceutical composition that comprises a compound of the general formula (5a′) or a salt or polymorph thereof, 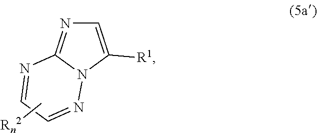
wherein
R¹ is an unsubstituted phenyl, a phenyl substituted with C₁-C₄-alkyl, F, Cl, Br, I, —CN, a substituted or unsubstituted biphenyl, or —(C═O)—R³, wherein R³ is a substituted or unsubstituted aryl or 5- to 6-membered heteroaryl, and
n of R²ₙ is 1 or 2, wherein each R² is independently a substituted or unsubstituted C₃-C₈ cycloalkyl, a substituted or unsubstituted C₁-C₆ alkyl, a substituted or unsubstituted C₁-C₆ alcohol, a substituted or unsubstituted 6-membered heteroaryl, a halogen, or —O—CH₂—R⁴, wherein R⁴ is a substituted or unsubstituted 5- or 6-membered heteroaryl,
wherein the administering is in an amount effective to treat the epileptic condition in the subject, and wherein the amount comprises a dose of the compound or a salt or polymorph thereof of from about 0.003 mg to about 1 mg per kg of a body weight of the subject per day.

40. A method of treating an epileptic condition in a subject, comprising administering to the subject a pharmaceutical composition that comprises a compound having a structure of 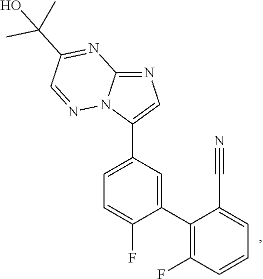 [SMILES: CC(C)(O)c1cnn2c(-c3ccc(F)c(-c4c(F)cccc4C#N)c3)cnc2n1]
or a salt or polymorph thereof, wherein the administering is in an amount effective to treat the epileptic condition in the subject, and wherein the amount comprises a dose of the compound or a salt or polymorph thereof of from about 0.0003 mg to about 1 mg per kg of a body weight of the subject per day.

41. The method of claim 40, wherein the compound, or the salt or polymorph thereof, is a phosphate salt or polymorph thereof or a sulfate salt or polymorph thereof.

42. (canceled)

43. The method of claim 40, wherein the compound, or a salt or polymorph thereof, is a phosphate polymorph, and wherein the phosphate polymorph exhibits an X-ray powder diffraction (XRPD) pattern having characteristic peak locations of at least three of the values selected from the following: about 6.4, 7.5, 10.2, 12.7, 13.3, 14.5, 16.0, 17.1, 17.4, 17.9, 18.5, 19.1, 19.7, 20.3, 20.9, 21.5, 22.6, 23.7, 26.2, 26.7, 26.9, 27.5, 28.4, 30.2, and 32.1±0.2 degrees, 2-theta, when measured using:
(a) an X-ray wavelength parameter of Cu: K-Alpha (λ=1.54179 Å); (b) an X-Ray tube voltage setting of 40 kV and current of 40 mA; (c) a scan scope of from about 3 degrees to about 40 degrees; (d) a sample rotation speed of about 15 rpm; and (e) a scanning rate of 10 degrees per minute.

44.-47. (canceled)

48. The method of claim 40, wherein the epileptic condition is Dravet syndrome, a focal seizure, a general epilepsy or a genetic epilepsy.

49.-50. (canceled)

51. The method of claim 40, wherein the compound, or a salt or polymorph thereof, is not a phosphate salt or polymorph thereof, and is not a sulfate salt or polymorph thereof.

52.-53. (canceled)

54. The method of claim 40, wherein the epileptic condition is selected from the following: Benign centrotemporal lobe epilepsy of childhood, Benign occipital epilepsy of childhood (BOEC), Autosomal dominant nocturnal frontal lobe epilepsy (ADNFLE), Primary reading epilepsy, Childhood absence epilepsy (CEA), Juvenile absence epilepsy, Juvenile myoclonic epilepsy (JME), Symptomatic localization-related epilepsies, Temporal lobe epilepsy (TLE), Frontal lobe epilepsy, Rasmussen's encephalitis, Cerebral palsy, Cerebral hypoxia, Down's syndrome, hypoxic-ischemic encephalopathy (HIE), West syndrome, Dravet syndrome, focal seizures, Progressive myoclonic epilepsies, or Lennox-Gastaut syndrome (LGS).

55. The method of claim 40, wherein the subject is a human.
